(19) United States
(12) Patent Application Publication
(10) Pub. No.: US 2026/0056211 A1
(43) Pub. Date: Feb. 26, 2026

(54) BIOMARKERS FOR CHRONIC FATIGUE SYNDROME AND LONG COVID AND USES THEREOF

(71) Applicant: BRISTOL-MYERS SQUIBB COMPANY, Princeton, NJ (US)

(72) Inventors: Sarah Ann HERSEY, Mendham, NJ (US); Zheng WANG, Ambler, PA (US); Tara Joy BASAVANHALLY, Hopewell Township, NJ (US); Gonzalo LOPEZ GARCIA, New York, NY (US); Yixin WANG, Basking Ridge, NJ (US)

(73) Assignee: BRISTOL-MYERS SQUIBB COMPANY, Princeton, NJ (US)

(21) Appl. No.: 18/869,706

(22) PCT Filed: May 26, 2023
(86) PCT No.: PCT/US2023/067555

§ 371 Date: Nov. 26, 2024

Related U.S. Application Data
(60) Provisional application No. 63/346,698, filed on May 27, 2022.

(51) Int. Cl. G01N 33/68; C12Q 1/6883

(52) U.S. Cl. CPC G01N 33/6893; C12Q 1/6883; C12Q 2600/158; G01N 2333/165; G01N 2800/306; G01N 2800/52

(57) ABSTRACT

The present disclosure provides methods and kits using certain biomarkers in predicting and monitoring post-viral syndromes (for instance, chronic fatigue syndrome (CFS) and/or long CO VID), selectively treating such syndromes, and assessing clinical sensitivity and therapeutic response to treatments. Wherein determining an expression level of a biomarker in a sample from the subject is disclosed.

### CROSS-REFERENCE TO RELATED APPLICATIONS

This application claims priority to U.S. Provisional Application No. 63/346,698 filed May 27, 2022, the content of which is incorporated by reference in its entirety herein.

### 1. FIELD

The present disclosure provides methods and kits using certain biomarkers in predicting and monitoring post-viral syndromes (for instance, chronic fatigue syndrome (CFS) and/or long COVID), selectively treating such syndromes, and assessing clinical sensitivity and therapeutic response to treatments.

### 2. BACKGROUND

Post-viral syndromes, which can include chronic fatigue syndrome (CFS) and long COVID, are complex conditions that can involve physical, cognitive, emotional, and neurological difficulties. These conditions can lead to symptoms including fatigue, post-exertional malaise, cognitive dysfunction, sensorimotor symptoms, headache, memory issues, insomnia, muscle aches, heart palpitations, shortness of breath, dizziness and balance issues, speech and language issues, joint pain, and tightness of chest. The symptoms can linger for weeks, months or longer after the viral infection has cleared. In some instances, affected patients become housebound and/or bedridden.

CFS, also referred to as myalgic encephalomyelitis (ME), is a debilitating condition that can lead to severe impairment of physical, psychological, cognitive, social, and occupational functions. Though the cause of CFS remains controversial, complicated, and incompletely understood, CFS can develop following a viral infection. So far, there is no clinical diagnostic test for CFS. Although some therapies have been used off-label to manage symptoms of CFS, limited, if any, specific therapies exist for CFS.

Some people who have been infected with the virus that causes COVID-19 can experience long-term effects from their infection, known as Long COVID. Long COVID is also known as Post-COVID Conditions, long-haul COVID, post-acute COVID-19, long-term effects of COVID, and chronic COVID. People with Long COVID can have a wide range of symptoms that can last weeks, months, or even years after infection.

There is still a need in the art for methods of identification, characterization, and treatment of CFS, long COVID and post-viral syndromes that are associated with fatigue.

### 3. SUMMARY OF THE INVENTION

In one aspect, provided herein is a method of identifying a subject having Chronic Fatigue Syndrome (CFS) or verifying CFS in a subject, the method comprising: (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; and (b) identifying or verifying the subject as having CFS if the expression level of the biomarker is higher than a reference expression level of the biomarker.

In some embodiments, the subject has reported chronic debilitating fatigue, unrefreshing sleep, mental and/or physical pain, neurological and cognitive impairment, and/or autoimmunity or immunodeficiencies.

In another aspect, provided herein is a method of determining severity of CFS in a subject, the method comprising: (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; (b) comparing the expression level of the biomarker with a reference expression level of the biomarker; and (c) determining the severity of CFS in the subject based on the comparison in step (b).

In some embodiments, the severity of CFS is determined to be severe if the expression level of the biomarker is higher than the reference expression level of the biomarker.

In some embodiments, the reference expression level of the biomarker is the expression level of the biomarker in a subject having mild CFS or a cohort of subjects having mild CFS.

In another aspect, provided herein is a method of identifying a subject who is likely or not likely to be responsive to a treatment of CFS or predicting the responsiveness of a subject to a treatment of CFS, the method comprising: (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; and (b) identifying or predicting the subject as being likely to be responsive to the treatment if the expression level of the biomarker is higher than a reference expression level of the biomarker.

In some embodiments, the method further comprises administering the treatment to the subject identified or predicted to be likely to be responsive to the treatment.

In another aspect, provided herein is a method of selectively treating a subject having or suspected of having CFS with a treatment, the method comprising: (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; (b) identifying or predicting the subject as being likely to be responsive to the treatment of CFS if the expression level of the biomarker is higher than a reference expression level of the biomarker; and (c) administering the treatment to the subject identified or predicted to be likely to be responsive to the treatment.

In some embodiments, the reference expression level of the biomarker is a predetermined expression level of the biomarker. In some embodiments, the reference expression level of the biomarker is the expression level of the biomarker in a subject who does not have CFS or a cohort of subjects not having CFS. In some embodiments, the reference expression level of the biomarker is the expression level of the biomarker in a healthy subject or a cohort of healthy subjects. In some embodiments, the reference expression level of the biomarker is the expression level of the biomarker in a subject having mild CFS or a cohort of subjects having mild CFS.

In some embodiments, the biomarker is HSPA8 or ABCE1, and optionally the reference expression level of the biomarker is the expression level of the biomarker in a healthy subject or a subject who does not have CFS, or a cohort of healthy subjects or subjects not having CFS.

In some embodiments, the method comprises determining the expression levels of two, three, four, five, or all biomarkers selected from the group consisting of HSPA8, ABCE1, IKZF2, IKZF3, BACH2 and CD3D. In some embodiments, the method comprises determining the expression levels of: (i) IKZF2 and at least one, two, three or four of IKZF3, ABCE1, BACH2, CD3D and HSPA8; (ii) IKZF3 and at least one, two, three or four of IKZF2, ABCE1, BACH2, CD3D and HSPA8; (iii) ABCE1 and at least one, two, three or four of IKZF2, IKZF3, BACH2, CD3D and HSPA8; (iv) BACH2 and at least one, two, three or four of IKZF2, IKZF3, ABCE1, CD3D and HSPA8; (v) CD3D and at least one, two, three or four of IKZF2, IKZF3, ABCE1, BACH2, and HSPA8; or (vi) HSPA8 and at least one, two, three or four of IKZF2, IKZF3, ABCE1, BACH2, and CD3D. In some embodiments, the method comprises determining the expression levels IKZF2, IKZF3, ABCE1, BACH2, CD3D and HSPA8.

In some embodiments, the method comprises comparing the expression level of each of the biomarkers with their respective reference expression level. In some embodiments, the method comprises obtaining a composite score based on the expression levels of the biomarkers and comparing the composite score with a reference score derived from the reference expression levels of the biomarkers.

In another aspect, provided herein is a method of determining or monitoring effectiveness of a treatment in a subject having CFS, the method comprising: (a) determining a first expression level of a biomarker in a first sample obtained from the subject before administering the treatment to the subject, wherein the biomarker is a cereblon (CRBN)-associated protein (CAP) selected from the group consisting of HSPA8, ABCE1, IKZF2, IKZF3, BACH2 and CD3D; (b) administering the treatment to the subject; (c) determining a second expression level of the biomarker in a second sample obtained from the subject after administering the treatment to the subject; and (d) determining the effectiveness of the treatment based on the comparison of the first expression level with the second expression level.

In some embodiments, the method comprises determining that the treatment is effective if the second expression level is lower than the first expression level. In some embodiments, the method further comprises determining or adjusting a dose of the treatment to the subject.

In another aspect, provided herein is a method of screening a treatment for effectiveness in treating CFS, the method comprising: (a) determining a first expression level of a biomarker in a sample before administering the treatment to the sample, wherein the biomarker is a cereblon (CRBN)-associated protein (CAP) selected from the group consisting of HSPA8, ABCE1, IKZF2, IKZF3, BACH2 and CD3D; (b) administering the treatment to the sample; (c) determining a second expression level of the biomarker in the sample after administering the treatment to the sample; (d) comparing the first expression level with the second expression level; and (e) selecting the treatment if the second expression level is lower than the first expression level.

In some embodiments, the biomarker is HSPA8 or ABCE1.

In some embodiments, the method comprises determining the first and second expression levels of two, three, four, five, or all biomarkers selected from the group consisting of HSPA8, ABCE1, IKZF2, IKZF3, BACH2 and CD3D. In some embodiments, the method comprises determining the first and second expression levels of: (i) IKZF2 and at least one, two, three or four of IKZF3, ABCE1, BACH2, CD3D and HSPA8; (ii) IKZF3 and at least one, two, three or four of IKZF2, ABCE1, BACH2, CD3D and HSPA8; (iii) ABCE1 and at least one, two, three or four of IKZF2, IKZF3, BACH2, CD3D and HSPA8; (iv) BACH2 and at least one, two, three or four of IKZF2, IKZF3, ABCE1, CD3D and HSPA8; (v) CD3D and at least one, two, three or four of IKZF2, IKZF3, ABCE1, BACH2, and HSPA8; or (vi) HSPA8 and at least one, two, three or four of IKZF2, IKZF3, ABCE1, BACH2, and CD3D. In some embodiments, the method comprises determining the first and second expression levels of all biomarkers in the group consisting of HSPA8, ABCE1, IKZF2, IKZF3, BACH2 and CD3D.

In some embodiments, the method comprises comparing the first expression level of each of the biomarkers with their respective second expression level. In some embodiments, the method comprises obtaining a first composite score based on the first expression levels of the biomarkers and a second composite score based on the second expression level of the biomarkers, and comparing the first composite score with the second composite score.

In some embodiments, the treatment comprises an immunomodulatory drug (IMiD). In some embodiments, the treatment comprises a celebron (CRBN) modulator or a compound capable of binding and/or inducing conformational change to CRBN. In some embodiments, the treatment comprises an agent that depletes B cells (e.g., an anti-CD20 antibody, e.g., rituximab).

In some embodiments, the CFS is associated with an autoimmune disease or a viral infection.

In another aspect, provided herein is a method of identifying a subject having long COVID or verifying long COVID in a subject, the method comprising: (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and (b) identifying or verifying the subject as having long COVID if the expression level of the biomarker is higher than a reference expression level of the biomarker.

In another aspect, provided herein is a method of identifying a subject who is likely or not likely to be responsive to a treatment of long COVID or predicting the responsiveness of a subject to a treatment of long COVID, the method comprising: (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and (b) identifying or predicting the subject as being likely to be responsive to the treatment if the expression level of the biomarker is higher than a reference expression level of the biomarker.

In some embodiments, the method further comprises administering the treatment to the subject identified or predicted to be likely to be responsive to the treatment.

In another aspect, provided herein is a method of selectively treating a subject having or suspected of having long COVID with a treatment, the method comprising: (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; (b) identifying or predicting the subject as being likely to be responsive to a treatment of long COVID if the expression level of the biomarker is higher than a reference expression level of the biomarker; and (c) administering the treatment to the subject identified or predicted to be likely to be responsive to the treatment.

In some embodiments, the reference expression level of the biomarker is a predetermined expression level of the biomarker. In some embodiments, the reference expression level of the biomarker is the expression level of the biomarker in a subject who does not have long COVID or a cohort of subjects not having long COVID. In some embodiments, the reference expression level of the biomarker is the expression level of the biomarker in a healthy subject or a cohort of healthy subjects. In some embodiments, the reference expression level of the biomarker is the expression level of the biomarker in a subject having acute COVID or a cohort of subjects having acute COVID.

In some embodiments, the biomarker is HSPA8 or IKZF3, optionally the reference expression level of the biomarker is the expression level of the biomarker in a healthy subject or a subject does not have long COVID, or a cohort of healthy subjects or subjects not having long COVID.

In some embodiments, the method comprises determining the expression levels of two, three, four, five, or all biomarkers selected from the group consisting of HSPA8, IKZF3, ABCE1, IKZF2, BACH2 and CD3D. In some embodiments, the method comprises determining the expression levels of: (i) IKZF2 and at least one, two, three or four of IKZF3, ABCE1, BACH2, CD3D and HSPA8; (ii) IKZF3 and at least one, two, three or four of IKZF2, ABCE1, BACH2, CD3D and HSPA8; (iii) ABCE1 and at least one, two, three or four of IKZF2, IKZF3, BACH2, CD3D and HSPA8; (iv) BACH2 and at least one, two, three or four of IKZF2, IKZF3, ABCE1, CD3D and HSPA8; (v) CD3D and at least one, two, three or four of IKZF2, IKZF3, ABCE1, BACH2, and HSPA8; or (vi) HSPA8 and at least one, two, three or four of IKZF2, IKZF3, ABCE1, BACH2, and CD3D. In some embodiments, the method comprises determining the expression levels HSPA8, IKZF3, ABCE1, IKZF2, BACH2 and CD3D.

In some embodiments, the method comprises comparing the expression level of each of the biomarkers with their respective reference expression level. In some embodiments, the method comprises obtaining a composite score based on the expression levels of the biomarkers and comparing the composite score with a reference score derived from the reference expression levels of the biomarkers.

In another aspect, provided herein is a method of determining or monitoring effectiveness of a treatment in a subject having long COVID, the method comprising: (a) determining a first expression level of a biomarker in a first sample obtained from the subject before administering the treatment to the subject, wherein the biomarker is a cereblon (CRBN)-associated protein (CAP) selected from the group consisting of HSPA8, IKZF3, ABCE1, IKZF2, BACH2 and CD3D; (b) administering the treatment to the subject; (c) determining a second expression level of the biomarker in a second sample obtained from the subject after administering the treatment to the subject; and (d) determining the effectiveness of the treatment based on the comparison of the first expression level with the second expression level.

In some embodiments, the method comprises determining that the treatment is effective if the second expression level is lower than the first expression level. In some embodiments, the method further comprises determining or adjusting a dose of the treatment to the subject.

In another aspect, provided herein is a method of screening a treatment for effectiveness in treating long COVID, the method comprising: (a) determining a first expression level of a biomarker in a sample before administering the treatment to the sample, wherein the biomarker is a cereblon (CRBN)-associated protein (CAP) selected from the group consisting of HSPA8, IKZF3, ABCE1, IKZF2, BACH2 and CD3D; (b) administering the treatment to the sample; (c) determining a second expression level of the biomarker in the sample after administering the treatment to the sample; (d) comparing the first expression level with the second expression level; and (e) selecting the treatment if the second expression level is lower than the first expression level.

In some embodiments, the biomarker is HSPA8 or IKZF3.

In some embodiments, the method comprises determining the first and second expression levels of two, three, four, five, or all biomarkers selected from the group consisting of HSPA8, IKZF3, ABCE1, IKZF2, BACH2 and CD3D. In some embodiments, the method comprises determining the first and second expression levels of: (i) IKZF2 and at least one, two, three or four of IKZF3, ABCE1, BACH2, CD3D and HSPA8; (ii) IKZF3 and at least one, two, three or four of IKZF2, ABCE1, BACH2, CD3D and HSPA8; (iii) ABCE1 and at least one, two, three or four of IKZF2, IKZF3, BACH2, CD3D and HSPA8; (iv) BACH2 and at least one, two, three or four of IKZF2, IKZF3, ABCE1, CD3D and HSPA8; (v) CD3D and at least one, two, three or four of IKZF2, IKZF3, ABCE1, BACH2, and HSPA8; or (vi) HSPA8 and at least one, two, three or four of IKZF2, IKZF3, ABCE1, BACH2, and CD3D. In some embodiments, the method comprises determining the first and second expression levels of all biomarkers in the group consisting of HSPA8, IKZF3, ABCE1, IKZF2, BACH2 and CD3D.

In some embodiments, the method comprises comparing the first expression level of each of the biomarkers with their respective second expression level. In some embodiments, the method comprises obtaining a first composite score based on the first expression levels of the biomarkers and a second composite score based on the second expression level of the biomarkers, and comparing the first composite score with the second composite score.

In some embodiments, the treatment comprises an immunomodulatory drug (IMiD). In some embodiments, the treatment comprises a CRBN modulator or a compound capable of binding and/or inducing conformational change to CRBN. In some embodiments, the treatment comprises an agent that depletes B cells (e.g., an anti-CD20 antibody, e.g., rituximab).

In some embodiments, the subject has had Coronavirus Disease 2019 (COVID-19).

In some embodiments, the expression level of the biomarker is determined by measuring the mRNA level of the biomarker. In some embodiments, the mRNA level is determined by using quantitative reverse-transcriptase PCR (RT-qPCR), microarray, Northern blot or RNA sequencing. In some embodiments, the expression level of the biomarker is determined by measuring the protein level of the biomarker. In some embodiments, the protein level of the biomarker is determined by using mass spectrometry (MS), liquid chromatography-tandem mass spectrometry (LC MS/MS), immunoassay, flow cytometry, immunohistochemistry, western blot, or enzyme-linked immunosorbent assay (ELISA).

In one aspect, provided herein is a kit for performing any of the preceding methods comprising an agent for determining the expression level of at least one biomarkers selected from the group consisting of HSPA8, IKZF3, ABCE1, IKZF2, BACH2 and CD3D.

In some embodiments, the kit further comprises a tool for obtaining the sample.

In some embodiments, the kit further comprises an instruction on interpreting the determined expression level.

In some embodiments, the kit further comprises the reference expression level of the biomarker.

### 4. BRIEF DESCRIPTION OF THE FIGURES

FIG. 1 depicts the data analysis workflow for Autoimmune Profile (AIP) gene expression data.

FIGS. 2A-2B depict the correlation of gene expression measurements between samples collected at two timepoints (FIG. 2A) and the correlation of gene expression measurements of house-keeping genes between two batches (FIG. 2B). Wilcoxon signed-rank tests were performed between replicates on both the raw and normalized relative florescent units (RFU) of three housekeeping genes (ACTB, GAPDH, TFRC). To assess potential batch effects, both batches were combined and principal component analysis (PCA) was performed to visualize clusters of normalization genes and immune module genes separately.

FIG. 3 depicts unsupervised Clustering Analysis (heat map) of AIP data showing the relationship between genes significantly up- or down-regulated in participant subsets with certain demographic or clinical attributes. Single columns represent patients and column annotations align to their associated clinical or demographic categories. Individual rows denote the 51 AIP panel genes which are then split by differentially expressed subgroups for that gene on the left portion of the heatmap.

FIGS. 4A-4H depict forest plots and bootstrapping t-test (FIG. 4A and FIG. 4B) with coefficient weights and individual gene plots (FIGS. 4C-4H) comparing between bed-ridden and non-bed-ridden CFS patients. FIG. 4A depicts a univariable bootstrapped analysis that identified six genes of interest (in the box) that were differentially expressed by CFS bedridden Status. The forest plot visually displays the mean coefficient values and 95% lower and upper confident interval boundaries for each gene. Coefficients which did not overlap with zero are indicated in the box. FIG. 4B depicts multivariable bootstrap analysis of six key genes that were differentially expressed by CFS bedridden status. The forest plot visually displays the mean coefficient values and 95% lower and upper confident interval boundaries for each gene. CFS bedridden patients were set as the baseline variable for comparison to CFS non-bedridden (lines with triangle), and control patients (lines with circle).

FIG. 5 depicts bedridden CFS patients with other autoimmune diseases. These patients displayed larger differential expression in the same six genes (IKZF2, IKZF3, ABCE1, BACH2, CD3D and HSPA8).

FIG. 6A depicts unsupervised clustering of urine organic acids based on metabolic categories. The heatmap is based on scaled and centered mmol values split by CFS bedridden status (top annotation) and clustered by metabolic category (right annotation). FIG. 6B depicts a summary of p-values and fold changes of the organic acids in normal vs. CFS (timepoint-1 only). FIGS. 6C-6F depict one-way analysis of raw values by cohort (CFS or normal) for various organic acids (xanthurenic acid, glycolic acid, pyruvic acid, and hippuric acid). FIG. 6G depicts the heat map of CFS bedridden vs. non-bedridden. FIG. 6H depicts a summary of p-values and fold changes in CFS bedridden vs. CFS non-bedridden (Timepoint-1 only). Vanilmandelic acid has p<0.05 and a fold change of 1.28. FIG. 6I depicts a one-way analysis of raw value by severity (bedridden or not) for vanilmandelic acid.

FIG. 7 depicts a summary of key biological functions of six genes identified by AIP.

FIG. 8 depicts similarity between ME/CFS and long-hauler COVID.

FIGS. 9A-9F depict individual box plots comparing the gene expression of ABCE1 (FIG. 9A), BACH2 (FIG. 9B), CD3D (FIG. 9C), HSPA8 (FIG. 9D), IKZF2 (FIG. 9E), and IKZF3 (FIG. 9F) between severe acute COVID patients (COVID) and matching normal healthy volunteer (NHV) donors. Wilcoxon signed-rank p-value was as indicated.

FIGS. 10A-10F depict individual box plots comparing the gene expression of ABCE1 (FIG. 10A), BACH2 (FIG. 10B), CD3D (FIG. 10C), HSPA8 (FIG. 10D), IKZF2 (FIG. 10E), and IKZF3 (FIG. 10F) between Long COVID patients (LCOVID) and normal healthy volunteer (NHV) donors. Wilcoxon signed-rank p-value was as indicated.

### 5. DETAILED DESCRIPTION OF THE INVENTION

The present disclosure is based, in part, on the findings that there was a striking similarity in symptoms between long COVID and CFS, and that the levels of certain cereblon (CRBN)-associated protein (CAP) biomarkers (e.g., mRNAs, cDNAs, or proteins of those biomarkers in FIG. 7) correlated with CFS and long COVID and severity thereof.

### 5.1 Definitions

Unless otherwise defined herein, technical and scientific terms used in the present description have the meanings that are commonly understood by those of ordinary skill in the art. Whenever appropriate, terms used in the singular will also include the plural and vice versa. In the event that any description of a term set forth conflicts with any document incorporated herein by reference, the description of the term set forth below shall control.

The term “biomarker” as used herein is a substance whose detection, amount, change, or any other characterization thereof indicates a particular biological state, such as a disease condition or progression thereof. In some embodiments, biomarkers can be determined individually. In other embodiments, several biomarkers can be measured simultaneously.

The term “expression” as used herein refers to the transcription from a gene to give an RNA nucleic acid molecule at least complementary in part to a region of one of the two nucleic acid strands of the gene. The term “expression” as used herein also refers to the translation from the RNA molecule to give a protein, a polypeptide, or a portion thereof.

The term “level” as used herein in connection with a biomarker refers to the amount, accumulation, or rate of a biomarker molecule (e.g., mRNA or protein expression of a gene or an organic acid). A level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. As used herein, the level can be an absolute amount of a molecule in a sample or a relative amount of the molecule, determined under steady-state or non-steady-state conditions. In some embodiments, an expression level of a biomarker is a normalized expression level. In some embodiments, a normalized expression level can be obtained by obtaining an expression level of a housekeeping gene (e.g., ACTB, GAPDH, or TFRC) and normalizing the level of expression of the biomarker against the level of expression of the housekeeping gene (e.g., ACTB, GAPDH, or TFRC) to determine a normalized level of expression of biomarker.

Any methods known in the art for normalization can be used herein. Generally speaking, normalization methods for gene expression data can be divided into three categories, i.e., data-driven reference, external reference, and entire gene set reference. For data-driven procedures, a subset of genes that do not vary or vary least across samples is first identified as the data driven housekeeping genes to normalize the data set. For external controls, external controls have been designed in a number of experiments, such as spike-in controls. These native controls can be used as the foreign reference for gene expression data normalization. For entire gene set, all genes in an experiment are used to derive a value or several values for data normalization. Several algorithms consider the whole genome as a reference for data normalization. Another exemplary method of normalization is “global” normalization, which means that an entire panel of genes (e.g., whole transcriptome) are used and the RFU value for the gene of interest is divided by the median or mean RFU value for the entire panel. In some embodiments, the normalization methods for gene expression profiling data assume that a majority of genes in the genome are equally expressed in each experimental unit and symmetrical distribution of genes between over- and under-expression. Non-normalized data provide an alternative way when the above mentioned assumptions to normalization do not apply, for example, when the data contain a large partition of differentially expressed genes.

Normalized values can be scaled. An exemplary scaling procedure for gene expression profiling data is provided below: the relation between gene expression data observed in different technology platforms cannot be assumed to be the same in their raw data. To compare the data across platforms, the range of observed data is assumed to be similar. For example, in scaling, the observed data of means can be assumed to be the same, the simplest function to scale the range of data of one platform to that of the other platform can be done based on a log scale. The scaling allows centering the mean data point as well as aligning the range of the measurements between the platforms, therefore enabling comparison of gene expression profiling data from different platforms. In a specific exemplary method, the raw RFU is used and divided by the geometric mean of a set of housekeeping genes. In one embodiment, the set of genes comprises or consists of the 3 housekeeping genes ACTB, GAPDH, and TFRC. Log 2 of the normalized values can be calculated, which scales the values in a range of −1 to 1. This allows for plotting all genes together in one graph and comparison.

The term “reference level” refers to a level of a biomarker (e.g., an expression level of a gene (e.g., mRNA or protein expression level) or a level of an organic acid) which is of interest for comparative purposes. A reference level of a biomarker (e.g., a reference expression level of a gene (e.g., mRNA or protein expression level) or a level of an organic acid) may be determined in the subject by any one of the methods provided herein. In some embodiments, the reference level of a biomarker is a predetermined level of said biomarker. In some embodiments, a predetermined expression level can be found in a public database. In some embodiments, the reference level is the expression level of a gene in blood. In some embodiments, the reference level is the level of an organic acid in urine. In some embodiments, the reference level of a biomarker is a level of said biomarker (e.g., an expression level of a gene (e.g., mRNA or protein expression level) or a level of an organic acid) in the same subject measured at a different time point or from a different sample. In some embodiments, the reference level of a biomarker is the level (e.g., an expression level of a gene (e.g., mRNA or protein expression level) or a level of an organic acid) of said biomarker in a corresponding tissue measured in a control subject (e.g., a healthy subject, a subject not having CFS, a subject having mild CFS, a subject not having long COVID, a subject having acute COVID). In some embodiments, the reference level of a biomarker is the level (e.g., a median or mean expression level of a gene (e.g., mRNA or protein expression level) or a median or mean level of an organic acid) of said biomarker in corresponding tissues measured in a cohort of subjects (e.g., a cohort of healthy subjects, a cohort of subject not having CFS, a cohort of subject having mild CFS (used interchangeably as moderate CFS herein), a cohort of subjects not having long COVID, a cohort of subjects having acute COVID). In some embodiments, the reference level of a biomarker (e.g., a reference expression level of a gene (e.g., mRNA or protein expression level) or a level of an organic acid) is the level of said biomarker in a sample obtained from a subject before the administration of a treatment and/or a compound to the subject.

The term “treat” or “treatment” or “treating” or “to treat” or “alleviate” or alleviation” or “alleviating” or “to alleviate” as used herein refers to therapeutic measures that aim to cure, slow down, lessen symptoms of, and/or halt progression of a pathologic condition or disorder.

As used herein, and unless otherwise specified, the term “therapeutically effective amount” of a compound is an amount sufficient to provide a therapeutic benefit in the treatment or management of a disease or disorder (e.g., CFS or long COVID), or to delay or minimize one or more symptoms associated with the presence of the a disease or disorder (e.g., CFS or long COVID). A therapeutically effective amount of a compound means an amount of a therapeutic agent, alone or in combination with other therapies, which provides a therapeutic benefit in the treatment or management of a disease or disorder (e.g., CFS or long COVID). The term “therapeutically effective amount” can encompass an amount that improves overall therapy, reduces or avoids symptoms or causes of a disease or disorder (e.g., CFS or long COVID), or enhances the therapeutic efficacy of another therapeutic agent. The term also refers to the amount of a compound that is sufficient to elicit the biological or medical response of a biological molecule (e.g., a protein, enzyme, RNA, DNA, or an organic acid), cell, tissue, system, animal, or human, which is being sought by a researcher, veterinarian, medical doctor, or clinician.

The term “prevent” or “prevention” or “preventing” as used herein refers to the partial or total inhibition of the development, recurrence, onset, or spread of a disease, disorder, or condition, or a symptom thereof in a subject.

The term “responsive” or “responsiveness” when used in reference to any one of the methods provided herein refers to the degree of effectiveness of a treatment in lessening or decreasing the symptoms of a disease, e.g., CFS or long COVID.

The term “predict” or “predicting” generally means to determine or tell in advance. The term “predicting” when used in reference to any one of the methods provided herein means that the likelihood of the outcome of the method is determined at the outset before certain action, e.g., a treatment is performed.

The term “obtaining” as used herein in connection with a level of a biomarker refers to an action or a step pertaining to getting information about the level of the biomarker. In some embodiments, the term “obtaining” includes a step of “determining”, “measuring”, “evaluating”, “assessing” and/or “assaying”. In some embodiments, “obtaining” involves ordering a determination, evaluation, assessment, and/or assay that is performed by a third party, or using the result of such a determination, evaluation, assessment, and/or assay. In some embodiments, the term “obtaining” also includes a step of getting a sample from a subject. In other embodiments, the term may also include steps necessary to prepare the sample in condition to be subject to an assay for measuring the nucleic acid or protein of a biomarker inside the sample.

The terms “determining”, “measuring”, “evaluating”, “assessing” and “assaying” are used interchangeably herein to refer to a form of measurement, including determining if an element is present or not. The measurement can be a quantitative and/or qualitative determination. “Determining the expression level of” can include measuring the amount of something present, as well as determining whether it is present or absent.

The term “monitor,” as used herein, generally refers to the overseeing, supervision, regulation, watching, tracking, or surveillance of an activity. For example, the term “monitoring the effectiveness of a compound” refers to tracking the effectiveness in treating a disease or disorder (e.g., CFS or long COVID) in a patient. The term “monitoring the effectiveness of a compound” also refers to tracking the effectiveness of treating CFS or long COVID (i.e., decreased fatigue) in a patient. Similarly, the term “monitoring,” when used in connection with patient compliance, either individually, or in a clinical trial, refers to the tracking or confirming that the patient is actually taking a drug being tested as prescribed. The monitoring can be performed, for example, by following the expression of mRNA or protein biomarkers or levels of urine organic acids.

The term “likely” or “likelihood” generally refers to an increase in the probability of an event. The term “likelihood” when used in reference to the effectiveness of a treatment in a subject generally contemplates an increased probability that progress or degree of the disease will decrease. The term “likelihood” when used in reference to the effectiveness of a treatment of CFS or long COVID can generally mean a decrease in the symptoms of CFS or long COVID (e.g., severe fatigue).

The term “sample” as used herein relates to a material or mixture of materials, for example, in fluid or solid form, containing one or more components of interest. A sample can be a biological sample obtained from a biological subject, including a sample of biological tissue or fluid origin, obtained, reached, or collected in vivo or in situ. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified.

Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%,15%,10%, 9%, 8%, 7%, 6%, 5%, 4%, 3%, 2%, 1%, 0.5%, or 0.05% of a given value or range.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

### 5.2 Biomarkers and Methods of Use Thereof

In one aspect, provided herein are methods of using the level of at least one cereblon-associated protein (CAP) biomarker (e.g., an expression level of a gene (e.g., mRNA or protein expression level)) identified herein that are associated with post-viral syndrome characterized by fatigue. In some embodiments, the fatigue is fatigue that has lasted for at least 3 months after infection with a virus. In some embodiments, the fatigue is fatigue that has lasted for at least 6 months after infection with a virus. In some embodiments, the fatigue is debilitating. In some embodiments, the fatigue interferes with activities of daily living. In some embodiments, the patient with the post-viral syndrome is housebound, bedridden, or both. In some embodiments, the post-viral syndrome comprises one more of the following: post-exertional malaise, cognitive dysfunction, sensorimotor symptoms, headache, memory issues, insomnia, muscle aches, heart palpitations, shortness of breath, dizziness and balance issues, speech and language issues, joint pain, tightness of chest. In certain embodiments, the post-viral syndrome is CFS. In certain embodiments, the post-viral syndrome is long COVID.

“Cereblon-associated protein” or “CAP” refers to a protein that interacts with or binds to CRBN, either directly or indirectly. In certain embodiments, the CAP is any protein that directly binds to cereblon, as well as any protein that is an indirect downstream effector of cereblon pathways. In certain embodiments, a “cereblon-associated protein” or “CAP” is a substrate of CRBN, for example, a protein substrate of the E3 ubiquitin ligase complex involving CRBN, or the downstream substrates thereof. In certain embodiments, the CAP is IKAROS Family Zinc Finger (IKZF2), IKAROS Family Zinc Finger 3 (IKZF3), ATP Binding Cassette Subfamily E Member 1 (ABCE1), BTB Domain and CNC Homology 2 (BACH2), CD3 Delta Subunit of T-cell Receptor (CD3D), or Heat Shock Protein Family A Member 8 (HSPA8).

In some embodiments, provided herein is a method of identifying a subject having post-viral syndrome, comprising obtaining an expression level of a biomarker in a sample from the subject, wherein the biomarker is a CAP selected from the group consisting of IKZF2, IKZF3, ABCE1, BACH2, CD3D and HSPA8, and identifying the subject as having the post-viral syndrome if the expression level of the biomarker is higher than a reference expression level of the biomarker.

In some embodiments, provided herein is a method of verifying post-viral syndrome in a subject, comprising obtaining an expression level of a biomarker in a sample from the subject, wherein the biomarker is a CAP selected from the group consisting of IKZF2, IKZF3, ABCE1, BACH2, CD3D and HSPA8; and verifying the subject as having the post-viral syndrome if the expression level of the biomarker is higher than a reference expression level of the biomarker.

In some embodiments, provided herein is a method of determining severity of post-viral syndrome in a subject comprising obtaining an expression level of a biomarker in a sample from the subject, wherein the biomarker is a CAP selected from the group consisting of IKZF2, IKZF3, ABCE1, BACH2, CD3D and HSPA8, and determining the severity of the post-viral syndrome in the subject based on the expression level. In some embodiments, the method further comprises comparing the expression level of the biomarker with a reference expression level of the biomarker. In some embodiments, a higher expression level of the biomarker indicates more severe post-viral syndrome.

In some embodiments, provided herein is a method of monitoring progress of post-viral syndrome in a subject comprising obtaining an expression level of a biomarker in a sample from the subject, wherein the biomarker is a CAP selected from the group consisting of IKZF2, IKZF3, ABCE1, BACH2, CD3D and HSPA8, and assessing the progress of post-viral syndrome based on the expression level of the biomarker. In certain embodiments, a higher expression level of the biomarker as compared to a reference expression level of the biomarker (e.g., the expression level of the biomarker at an earlier timepoint) indicates that the post-viral syndrome progresses. In certain embodiments, a lower expression level of the biomarker as compared to the reference expression level of the biomarker (e.g., the expression level of the biomarker at an earlier timepoint) indicates that the post-viral syndrome regresses.

In some embodiments, provided herein is a method of identifying a subject who is likely or not likely to be responsive to a treatment of post-viral syndrome or predicting the responsiveness of a subject to a treatment of post-viral syndrome comprising obtaining an expression level of a biomarker in a sample from the subject, wherein the biomarker is a CAP selected from the group consisting of IKZF2, IKZF3, ABCE1, BACH2, CD3D and HSPA8; and identifying or predicting the subject as being likely to be responsive to a treatment of post-viral syndrome if the expression level of the biomarker is higher than a reference expression level of the biomarker.

In some embodiments, provided herein is a method of selectively treating a subject having or suspected of having post-viral syndrome with a treatment, the method comprising obtaining an expression level of a biomarker in a sample from the subject, wherein the biomarker is a CAP selected from the group consisting of IKZF2, IKZF3, ABCE1, BACH2, CD3D and HSPA8; identifying or predicting the subject as being likely to be responsive to a treatment of post-viral syndrome if the expression level of the biomarker is higher than a reference expression level of the biomarker; and administering the treatment to the subject identified or predicted to be likely to be responsive to the treatment.

In some embodiments, the reference expression level of the biomarker is a predetermined expression level of the biomarker from a public database. In some embodiments, the reference expression level of the biomarker is an expression level of the biomarker in a subject who does not have the post-viral syndrome or a healthy subject. In some embodiments, the reference expression level of the biomarker is an expression level of the biomarker in a subject having mild post-viral syndrome or having acute viral infection. In some embodiments, the reference expression level of the biomarker is an expression level of the biomarker determined based on a cohort of subjects (e.g., a cohort of healthy subjects, a cohort of subjects not having post-viral syndrome, a cohort of subject having mild post-viral syndrome, or a cohort of subject having acute viral infection). In some embodiments, the reference expression level of the biomarker is a median or a mean expression level of the biomarker of the expression levels of the biomarker in a cohort of subjects (e.g., a cohort of healthy subjects, a cohort of subjects not having post-viral syndrome, a cohort of subject having mild post-viral syndrome, or a cohort of subject having acute viral infection).

In certain embodiments, the method comprises using two or more biomarkers selected from the group consisting of IKZF2, IKZF3, ABCE1, BACH2, CD3D and HSPA8. In some embodiments, when multiple biomarkers are used, the expression level of each biomarker is compared with a reference expression level of that biomarker. In other embodiments, when multiple biomarkers are used, a composite score is calculated based on the multiple biomarkers and compared with a reference composite score. In some embodiments, a composite score is calculated using the Median Z-Score method. In other embodiments, a composite score is calculated using the Single-Sample Gene Set Enrichment (ssGSEA) method.

In some embodiments, provided herein is a method of determining or monitoring effectiveness of a treatment in a subject having post-viral syndrome comprising obtaining a first expression level of a biomarker in a first sample from the subject, wherein the biomarker is a CAP selected from the group consisting of IKZF2, IKZF3, ABCE1, BACH2, CD3D and HSPA8; administering the treatment to the subject; obtaining a second expression level of the biomarker in a second sample obtained from the subject after administering the treatment to the subject; and determining the effectiveness of the treatment based on the comparison of the first expression level with the second expression level. In some embodiments, the method comprises determining that the treatment is effective if the second expression level is lower than the first expression level. In some embodiments, the method comprises determining that the treatment is not effective if the second expression level is not lower than the first expression level. In some embodiments, the method comprises determining or adjusting (e.g., increasing) the dose of the treatment or administering a different treatment to the subject if the second expression level is not lower than the first expression level

In some embodiments, provided herein is a method of screening a treatment for effectiveness in treating post-viral syndrome, the method comprising: (a) determining a first expression level of a biomarker in a sample before administering the compound to the sample, wherein the biomarker is a cereblon (CRBN)-associated protein (CAP) selected from the group consisting of HSPA8, IKZF3, ABCE1, IKZF2, BACH2 and CD3D; (b) administering the treatment to the sample; (c) determining a second expression level of the biomarker in the sample after administering the treatment to the sample; (d) comparing the first expression level with the second expression level; and (e) selecting the treatment if the second expression level is lower than the first expression level.

In certain embodiments, the method comprises using two or more biomarkers selected from the group consisting of IKZF2, IKZF3, ABCE1, BACH2, CD3D and HSPA8. In some embodiments, when multiple biomarkers are used, the second expression level of each biomarker is compared with a first expression level of that biomarker, and the method comprises determining that the treatment is effective if the second expression level of each biomarker is lower than the first expression level of that biomarker. In other embodiments, when multiple biomarkers are used, a composite score is calculated based on the multiple biomarkers and compared with a reference composite score. In some embodiments, a composite score is calculated using the Median Z-Score method. In other embodiments, a composite score is calculated using the Single-Sample Gene Set Enrichment (ssGSEA) method.

In some embodiments, the treatment of post-viral syndrome disclosed herein comprises an immunomodulatory drug (IMiD) as disclosed in Section 5.2. In some embodiments, the treatment post-viral syndrome comprises a celebron (CRBN) modulator or a compound capable of binding and/or inducing conformational change to CRBN as disclosed in Section 5.2. In some embodiments, the treatment post-viral syndrome comprises an agent that depletes B cells (e.g., an anti-CD20 antibody, e.g., rituximab) as disclosed in Section 5.2.

### 5.2.1 Biomarkers for CFS

“Chronic Fatigue Syndrome” or “CFS” as used herein refers to a disease with a symptom of fatigue lasting over a period of time, for example, for at least 4 weeks. In some embodiments, the period of time is at least 6 weeks. In some embodiments, the period of time is at least 2 months, 3 months, 4 months, 5 months, or 6 months. In some embodiments, the period of time is at least 6 months. The symptom of fatigue can be persistent or intermittent over this period of time. In some embodiments, the symptom of fatigue is persistent over said period of time. In some embodiments, the symptom of fatigue results in the patient being bedridden. In some embodiments, the disease comprises a combination of symptoms, which can include one or more of the following: debilitating fatigue, post-exertional malaise, unrefreshing sleep or sleep disturbance (or both), and/or cognitive difficulties. In some embodiments, the combination of symptoms includes debilitating fatigue, post-exertional malaise, unrefreshing sleep or sleep disturbance (or both), and cognitive difficulties. In some embodiments, this combination of symptoms is present for a period of time disclosed herein. In some embodiments, the period of time is at least six weeks in adults and at least four weeks in children. Most often, the symptom of fatigue lasts more than six months. In certain cases, the disease may have various other symptoms such as unrefreshing sleep, mental and physical pain, neurological and cognitive impairment, as well as autoimmunity or immunodeficiencies.

In some embodiments, CFS is a post-viral syndrome. In some embodiments, CFS occurs in the absence of a viral infection, and thus is not a post-viral syndrome.

In some embodiments, provided herein is a method of identifying a subject having CFS, comprising obtaining an expression level of a biomarker in a sample from the subject, wherein the biomarker is a CAP selected from the group consisting of IKZF2, IKZF3, ABCE1, BACH2, CD3D and HSPA8, and identifying the subject as having CFS if the expression level of the biomarker is higher than a reference expression level of the biomarker. In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8.

In some embodiments, the reference expression level of the biomarker is a predetermined expression level of the biomarker. In some embodiments, the reference expression level of the biomarker is a predetermined expression level of the biomarker obtained from a public database. In some embodiments, the reference expression level of the biomarker is an expression level of the biomarker in a healthy subject or a subject who does not have CFS. In some embodiments, the reference expression level of the biomarker is an expression level of the biomarker in a subject having moderate CFS. In some embodiments, the reference expression level of the biomarker is an expression level of the biomarker determined based on a cohort of subjects (e.g., a cohort of healthy subjects, a cohort of subjects not having CFS, or a cohort of subjects having moderate CFS). In some embodiments, the reference expression level of the biomarker is a median or a mean expression level of the biomarker of the expression levels of the biomarker in a cohort of subjects (e.g., a cohort of healthy subjects, a cohort of subjects not having CFS, a cohort of subjects having moderate CFS). In some embodiments, the biomarker is HSPA8 or ABCE1 and the reference expression level of the biomarker is the expression level of the biomarker in a healthy subject or a subject does not have CFS, or a cohort of healthy subjects or subjects not having CFS.

In some embodiments, the method comprises identifying the subject as having CFS if the expression level of the biomarker is at least 5% higher than a reference expression level of the biomarker. In some embodiments, the method comprises identifying the subject as having CFS if the expression level of the biomarker is at least 10% higher than a reference expression level of the biomarker. In some embodiments, the method comprises identifying the subject as having CFS if the expression level of the biomarker is at least 20% higher than a reference expression level of the biomarker. In some embodiments, the method comprises identifying the subject as having CFS if the expression level of the biomarker is at least 30% higher than a reference expression level of the biomarker. In some embodiments, the method comprises identifying the subject as having CFS if the expression level of the biomarker is at least 40% higher than a reference expression level of the biomarker. In some embodiments, the method comprises identifying the subject as having CFS if the expression level of the biomarker is at least 50% higher than a reference expression level of the biomarker. In some embodiments, the method comprises identifying the subject as having CFS if the expression level of the biomarker is at least 60% higher than a reference expression level of the biomarker. In some embodiments, the method comprises identifying the subject as having CFS if the expression level of the biomarker is at least 70% higher than a reference expression level of the biomarker. In some embodiments, the method comprises identifying the subject as having CFS if the expression level of the biomarker is at least 80% higher than a reference expression level of the biomarker. In some embodiments, the method comprises identifying the subject as having CFS if the expression level of the biomarker is at least 90% higher than a reference expression level of the biomarker. In some embodiments, the method comprises identifying the subject as having CFS if the expression level of the biomarker is at least 2 fold of a reference expression level of the biomarker. In some embodiments, the method comprises identifying the subject as having CFS if the expression level of the biomarker is at least 3 fold of a reference expression level of the biomarker. In some embodiments, the method comprises identifying the subject as having CFS if the expression level of the biomarker is at least 4 fold of a reference expression level of the biomarker. In some embodiments, the method comprises identifying the subject as having CFS if the expression level of the biomarker is at least 5 fold of a reference expression level of the biomarker. In some embodiments, the method comprises identifying the subject as having CFS if the expression level of the biomarker is at least 6 fold of a reference expression level of the biomarker. In some embodiments, the method comprises identifying the subject as having CFS if the expression level of the biomarker is at least 7 fold of a reference expression level of the biomarker. In some embodiments, the method comprises identifying the subject as having CFS if the expression level of the biomarker is at least 8 fold of a reference expression level of the biomarker. In some embodiments, the method comprises identifying the subject as having CFS if the expression level of the biomarker is at least 9 fold of a reference expression level of the biomarker. In some embodiments, the method comprises identifying the subject as having CFS if the expression level of the biomarker is at least 10 fold of a reference expression level of the biomarker. The expression level of a biomarker can be determined using any known method in the art, and exemplary methods are described in more detail in Section 5.3 below. In some embodiments, an expression level of the biomarker is determined to be higher than a reference level if the level is higher (e.g., statistically significantly higher) than the reference level as observed according to a measurement assay.

In certain embodiments, the method comprises using two or more biomarkers selected from the group consisting of IKZF2, IKZF3, ABCE1, BACH2, CD3D and HSPA8 to identify a subject having CFS. In certain embodiments, the method comprises using three or more biomarkers selected from the group consisting of IKZF2, IKZF3, ABCE1, BACH2, CD3D and HSPA8 to identify a subject having CFS. In certain embodiments, the method comprises using four or more biomarkers selected from the group consisting of IKZF2, IKZF3, ABCE1, BACH2, CD3D and HSPA8 to identify a subject having CFS. In certain embodiments, the method comprises using five or more biomarkers selected from the group consisting of IKZF2, IKZF3, ABCE1, BACH2, CD3D and HSPA8 to identify a subject having CFS. In certain embodiments, the method comprises using all biomarkers selected from the group consisting of IKZF2, IKZF3, ABCE1, BACH2, CD3D and HSPA8 to identify a subject having CFS. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined. In some embodiments, when multiple biomarkers are used, the expression level of each biomarker is compared with a reference expression level of that biomarker, and the method comprises identifying the subject as having CFS if the expression level of each biomarker is higher than the reference expression level of that biomarker. In other embodiments, when multiple biomarkers are used, a composite score is calculated based on the multiple biomarkers and compared with a reference composite score. In some embodiments, the method comprises identifying the subject as having CFS if the composite score is higher than the reference composite score. In some embodiments, a composite score is calculated using the Median Z-Score method. Briefly, Median Z-Scores are derived by first calculating the mean of each gene from all samples within a gene expression matrix. The mean is then subtracted from each corresponding gene for all samples and then scaling is performed by dividing the values by their standard deviations. The median scaled value from multiple genes of interest comprises the composite score. Another exemplary method for calculating a composite score is the Single-Sample Gene Set Enrichment (ssGSEA) method.

Single-sample gene scores represent the degree to which the genes in a particular gene set are coordinately up- or down-regulated within a sample. The score is calculated by adjusting a running-sum statistic based on a decreasing walk through a ranked expression list. The enrichment score is the maximum deviation from zero encountered in the walk; it corresponds to a weighted Kolmogorov-Smirnov-like statistic (see, e.g., Subramanian et al., PNAS, 102 (43): 15545-15550 (2005); and Barbie et al., Nature, 462 (7269): 108-112).

In other embodiments, provided herein is a method of verifying CFS in a subject, comprising obtaining an expression level of a biomarker in a sample from the subject, wherein the biomarker is a CAP selected from the group consisting of IKZF2, IKZF3, ABCE1, BACH2, CD3D and HSPA8; and verifying the subject as having CFS if the expression level of the biomarker is higher than a reference expression level of the biomarker. In some embodiments, the subject has reported a symptom related to CFS. Thus, in some embodiments, provided herein is a method of verifying CFS in a subject comprising selecting a subject who has reported a symptom related to CFS; obtaining an expression level of a biomarker in a sample from the subject, wherein the biomarker is a CAP selected from the group consisting of IKZF2, IKZF3, ABCE1, BACH2, CD3D and HSPA8; and verifying the subject as having CFS if the expression level of the biomarker is higher than a reference expression level of the biomarker. Exemplary symptoms related to CFS include but are not limited to chronic debilitating fatigue, unrefreshing sleep, mental and/or physical pain, neurological and cognitive impairment, and/or autoimmunity or immunodeficiencies. In some embodiment, the biomarker is IKZF2. In some embodiment, the biomarker is IKZF3. In some embodiment, the biomarker is ABCE1. In some embodiment, the biomarker is BACH2. In some embodiment, the biomarker is CD3D. In some embodiment, the biomarker is HSPA8.

In some embodiments, the reference expression level of the biomarker is a predetermined expression level of the biomarker from a public database. In some embodiments, the reference expression level of the biomarker is an expression level of the biomarker in a subject who does not have CFS. In some embodiments, the reference expression level of the biomarker is an expression level of the biomarker in a subject having moderate CFS. In some embodiments, the reference expression level of the biomarker is an expression level of the biomarker determined based on a cohort of subjects (e.g., a cohort of healthy subjects, a cohort of subjects not having CFS, or a cohort of subject having moderate CFS). In some embodiments, the reference expression level of the biomarker is a median or a mean expression level of the biomarker of the expression levels of the biomarker in a cohort of subjects (e.g., a cohort of healthy subjects, a cohort of subjects not having CFS, or a cohort of subjects having moderate CFS). In some embodiments, the biomarker is HSPA8 or ABCE1 and the reference expression level of the biomarker is the expression level of the biomarker in a healthy subject or a subject does not have CFS, or a cohort of healthy subjects or subjects not having CFS. In some embodiments, the biomarker is HSPA8 or ABCE1 and the reference expression level of the biomarker is the expression level of the biomarker in a healthy subject or a subject does not have CFS, or a cohort of healthy subjects or subjects not having CFS.

In some embodiments, the method comprises verifying the subject as having CFS if the expression level of the biomarker is at least 5% higher than a reference expression level of the biomarker. In some embodiments, the method comprises verifying the subject as having CFS if the expression level of the biomarker is at least 10% higher than a reference expression level of the biomarker. In some embodiments, the method comprises verifying the subject as having CFS if the expression level of the biomarker is at least 20% higher than a reference expression level of the biomarker. In some embodiments, the method comprises verifying the subject as having CFS if the expression level of the biomarker is at least 30% higher than a reference expression level of the biomarker. In some embodiments, the method comprises verifying the subject as having CFS if the expression level of the biomarker is at least 40% higher than a reference expression level of the biomarker. In some embodiments, the method comprises verifying the subject as having CFS if the expression level of the biomarker is at least 50% higher than a reference expression level of the biomarker. In some embodiments, the method comprises verifying the subject as having CFS if the expression level of the biomarker is at least 60% higher than a reference expression level of the biomarker. In some embodiments, the method comprises verifying the subject as having CFS if the expression level of the biomarker is at least 70% higher than a reference expression level of the biomarker. In some embodiments, the method comprises verifying the subject as having CFS if the expression level of the biomarker is at least 80% higher than a reference expression level of the biomarker. In some embodiments, the method comprises verifying the subject as having CFS if the expression level of the biomarker is at least 90% higher than a reference expression level of the biomarker. In some embodiments, the method comprises verifying the subject as having CFS if the expression level of the biomarker is at least 2 fold of a reference expression level of the biomarker. In some embodiments, the method comprises verifying the subject as having CFS if the expression level of the biomarker is at least 3 fold of a reference expression level of the biomarker. In some embodiments, the method comprises verifying the subject as having CFS if the expression level of the biomarker is at least 4 fold of a reference expression level of the biomarker. In some embodiments, the method comprises verifying the subject as having CFS if the expression level of the biomarker is at least 5 fold of a reference expression level of the biomarker. In some embodiments, the method comprises verifying the subject as having CFS if the expression level of the biomarker is at least 6 fold of a reference expression level of the biomarker. In some embodiments, the method comprises verifying the subject as having CFS if the expression level of the biomarker is at least 7 fold of a reference expression level of the biomarker. In some embodiments, the method comprises verifying the subject as having CFS if the expression level of the biomarker is at least 8 fold of a reference expression level of the biomarker. In some embodiments, the method comprises verifying the subject as having CFS if the expression level of the biomarker is at least 9 fold of a reference expression level of the biomarker. In some embodiments, the method comprises verifying the subject as having CFS if the expression level of the biomarker is at least 10 fold of a reference expression level of the biomarker. The expression level of a biomarker can be determined using any known method in the art, and exemplary methods are described in more detail in Section 5.3 below. In some embodiments, a level is determined to be higher than a reference level if the level is higher (e.g., statistically significantly higher) than the reference level as observed according to a measurement assay.

In certain embodiments, the method comprises using two or more biomarkers selected from the group consisting of IKZF2, IKZF3, ABCE1, BACH2, CD3D and HSPA8 to verify a subject having CFS. In certain embodiments, the method comprises using three or more biomarkers selected from the group consisting of IKZF2, IKZF3, ABCE1, BACH2, CD3D and HSPA8 to verify a subject having CFS. In certain embodiments, the method comprises using four or more biomarkers selected from the group consisting of IKZF2, IKZF3, ABCE1, BACH2, CD3D and HSPA8 to verify a subject having CFS. In certain embodiments, the method comprises using five or more biomarkers selected from the group consisting of IKZF2, IKZF3, ABCE1, BACH2, CD3D and HSPA8 to verify a subject having CFS. In certain embodiments, the method comprises using all biomarkers selected from the group consisting of IKZF2, IKZF3, ABCE1, BACH2, CD3D and HSPA8 to verify a subject having CFS. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined. In some embodiments, when multiple biomarkers are used, the expression level of each biomarker is compared with a reference expression level of that biomarker, and the method comprises verifying the subject as having CFS if the expression level of each biomarker is higher than the reference expression level of that biomarker. In other embodiments, when multiple biomarkers are used, a composite score is calculated based on the multiple biomarkers and compared with a reference composite score. In some embodiments, the method comprises verifying the subject as having CFS if the composite score is higher than the reference composite score. In some embodiments, a composite score is calculated using the Median Z-Score method. In other embodiments, a composite score is calculated using the Single-Sample Gene Set Enrichment (ssGSEA) method.

The present disclosure is also based in part on the finding that the expression level of certain CAP biomarker is associated with severity of CFS. Thus, in other embodiments, provided herein is a method of determining severity of CFS in a subject comprising obtaining an expression level of a biomarker in a sample from the subject, wherein the biomarker is a CAP selected from the group consisting of IKZF2, IKZF3, ABCE1, BACH2, CD3D and HSPA8, and determining the severity of CFS in the subject based on the expression level. In some embodiments, the method further comprises comparing the expression level of the biomarker with a reference expression level of the biomarker. In some embodiments, a higher expression level of the biomarker indicates more severe CFS. “Severe CFS”, as used herein, refers to a type of CFS where the patients are affected severely and are housebound and/or bedridden. In certain embodiments, the severe CFS patients are housebound and/or bedridden most of the time. In certain embodiments, the severe CFS patients (e.g., very severe CFS patients) are totally bedridden and need help with basic activities including nutrition and hydration. “Mild CFS” and “moderate CFS” are used interchangeably herein and refer to a type of CFS where the patients are not housebound or bedridden.

In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8.

In some embodiments, the reference expression level of the biomarker is a predetermined expression level of the biomarker from a public database. In some embodiments, the reference expression level of the biomarker is an expression level of the biomarker in a healthy subject or a subject who does not have CFS. In some embodiments, the reference expression level of the biomarker is an expression level of the biomarker determined based on a cohort of healthy subjects. In some embodiments, the reference expression level of the biomarker is an expression level of the biomarker in a subject whose severity of CFS has been determined and known. In some embodiments, the reference expression level of the biomarker is an expression level of the biomarker in a subject having moderate CFS or a cohort of subjects having moderate CFS. In some embodiments, the reference expression level of the biomarker is a median or a mean expression level of the biomarker of the expression levels of the biomarker in a cohort of subjects (e.g., a cohort of healthy subjects, a cohort of subjects having moderate CFS, or a cohort of subjects whose severity of CFS has been determined and known).

In some embodiments, the biomarker is HSPA8 or ABCE1 and the reference expression level of the biomarker is the expression level of the biomarker in a healthy subject or a subject does not have CFS, or a cohort of healthy subjects or subjects not having CFS. In some embodiments, the biomarker is selected from the group consisting of IKZF2, IKZF3, ABCE1, BACH2, CD3D and HSPA8, and the reference expression level of the biomarker is the expression level of the biomarker in a subject having mild CFS or a cohort of subjects having mild CFS.

In yet other embodiments, provided herein is a method of monitoring progress of CFS in a subject comprising obtaining an expression level of a biomarker in a sample from the subject, wherein the biomarker is a CAP selected from the group consisting of IKZF2, IKZF3, ABCE1, BACH2, CD3D and HSPA8, and assessing the progress of CFS based on the expression level of the biomarker. In some embodiments, the expression level of the biomarker is obtained at two or more timepoints, e.g., periodically. In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8. In some embodiments, the reference expression level of the biomarker is an expression level of the biomarker in the same subject at an earlier timepoint. In certain embodiments, a higher expression level of the biomarker as compared to the reference expression level of the biomarker (e.g., the expression level of the biomarker at an earlier timepoint) indicates that the CFS progresses. In certain embodiments, a lower expression level of the biomarker as compared to the reference expression level of the biomarker (e.g., the expression level of the biomarker at an earlier timepoint) indicates that the CFS regresses.

The expression level of a biomarker can be determined using any known method in the art, and exemplary methods are described in more detail in Section 5.3 below. In some embodiments, a level is determined to be higher than a reference level if the level is higher (e.g., statistically significantly higher) than the reference level as observed according to a measurement assay. In some embodiments, a level is determined to be lower than a reference level if the level is lower (e.g., statistically significant) than the reference level as observed according to a measurement assay.

In certain embodiments, the method comprises using two or more biomarkers selected from the group consisting of IKZF2, IKZF3, ABCE1, BACH2, CD3D and HSPA8. In certain embodiments, the method comprises using three or more biomarkers selected from the group consisting of IKZF2, IKZF3, ABCE1, BACH2, CD3D and HSPA8. In certain embodiments, the method comprises using four or more biomarkers selected from the group consisting of IKZF2, IKZF3, ABCE1, BACH2, CD3D and HSPA8. In certain embodiments, the method comprises using five or more biomarkers selected from the group consisting of IKZF2, IKZF3, ABCE1, BACH2, CD3D and HSPA8. In certain embodiments, the method comprises using all biomarkers selected from the group consisting of IKZF2, IKZF3, ABCE1, BACH2, CD3D and HSPA8. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined. In some embodiments, when multiple biomarkers are used, the expression level of each biomarker is compared with a reference level of that biomarker. In other embodiments, when multiple biomarkers are used, a composite score is calculated based on the multiple biomarkers and compared with a reference composite score. In some embodiments, a composite score is calculated using the Median Z-Score method. In other embodiments, a composite score is calculated using the Single-Sample Gene Set Enrichment (ssGSEA) method.

In another aspect, the biomarkers provided herein are used to predict a subject's responsiveness to a treatment to CFS. In some embodiments, provided herein is a method of identifying a subject who is likely or not likely to be responsive to a treatment of CFS or predicting the responsiveness of a subject to a treatment of CFS comprising obtaining an expression level of a biomarker in a sample from the subject, wherein the biomarker is a CAP selected from the group consisting of IKZF2, IKZF3, ABCE1, BACH2, CD3D and HSPA8; and identifying or predicting the subject as being likely to be responsive to a treatment of CFS if the expression level of the biomarker is higher than a reference expression level of the biomarker. In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8.

In some embodiments, the reference expression level of the biomarker is a predetermined expression level of the biomarker obtained from a public database. In some embodiments, the reference expression level of the biomarker is an expression level of the biomarker in a healthy subject or a subject who does not have CFS. In some embodiments, the reference expression level of the biomarker is an expression level of the biomarker in a subject having moderate CFS. In some embodiments, the reference expression level of the biomarker is an expression level of the biomarker determined based on a cohort of subjects (e.g., a cohort of healthy subjects, a cohort of subjects not having CFS, or a cohort of subjects having moderate CFS). In some embodiments, the reference expression level of the biomarker is a median or a mean expression level of the biomarker of the expression levels of the biomarker in a cohort of subjects (e.g., a cohort of healthy subjects, a cohort of subjects not having CFS, or a cohort of subjects having moderate CFS). In some embodiments, the biomarker is HSPA8 or ABCE1 and the reference expression level of the biomarker is the expression level of the biomarker in a healthy subject or a subject does not have CFS, or a cohort of healthy subjects or subjects not having CFS.

In some embodiments, the method comprises identifying or predicting the subject as likely to be responsive to a treatment to CFS if the expression level of the biomarker is at least 5% higher than a reference expression level of the biomarker. In some embodiments, the method comprises identifying or predicting the subject as likely to be responsive to a treatment to CFS if the expression level of the biomarker is at least 10% higher than a reference expression level of the biomarker. In some embodiments, the method comprises identifying or predicting the subject as likely to be responsive to a treatment to CFS if the expression level of the biomarker is at least 20% higher than a reference expression level of the biomarker. In some embodiments, the method comprises identifying or predicting the subject as likely to be responsive to a treatment to CFS if the expression level of the biomarker is at least 30% higher than a reference expression level of the biomarker. In some embodiments, the method comprises identifying or predicting the subject as likely to be responsive to a treatment to CFS if the expression level of the biomarker is at least 40% higher than a reference expression level of the biomarker. In some embodiments, the method comprises identifying or predicting the subject as likely to be responsive to a treatment to CFS if the expression level of the biomarker is at least 50% higher than a reference expression level of the biomarker. In some embodiments, the method comprises identifying or predicting the subject as likely to be responsive to a treatment to CFS if the expression level of the biomarker is at least 60% higher than a reference expression level of the biomarker. In some embodiments, the method comprises identifying or predicting the subject as likely to be responsive to a treatment to CFS if the expression level of the biomarker is at least 70% higher than a reference expression level of the biomarker. In some embodiments, the method comprises identifying or predicting the subject as likely to be responsive to a treatment to CFS if the expression level of the biomarker is at least 80% higher than a reference expression level of the biomarker. In some embodiments, the method comprises identifying or predicting the subject as likely to be responsive to a treatment to CFS if the expression level of the biomarker is at least 90% higher than a reference expression level of the biomarker. In some embodiments, the method comprises identifying or predicting the subject as likely to be responsive to a treatment to CFS if the expression level of the biomarker is at least 2 fold of a reference expression level of the biomarker. In some embodiments, the method comprises identifying or predicting the subject as likely to be responsive to a treatment to CFS if the expression level of the biomarker is at least 3 fold of a reference expression level of the biomarker. In some embodiments, the method comprises identifying or predicting the subject as likely to be responsive to a treatment to CFS if the expression level of the biomarker is at least 4 fold of a reference expression level of the biomarker. In some embodiments, the method comprises identifying or predicting the subject as likely to be responsive to a treatment to CFS if the expression level of the biomarker is at least 5 fold of a reference expression level of the biomarker. In some embodiments, the method comprises identifying or predicting the subject as likely to be responsive to a treatment to CFS if the expression level of the biomarker is at least 6 fold of a reference expression level of the biomarker. In some embodiments, the method comprises identifying or predicting the subject as likely to be responsive to a treatment to CFS if the expression level of the biomarker is at least 7 fold of a reference expression level of the biomarker. In some embodiments, the method comprises identifying or predicting the subject as likely to be responsive to a treatment to CFS if the expression level of the biomarker is at least 8 fold of a reference expression level of the biomarker. In some embodiments, the method comprises identifying or predicting the subject as likely to be responsive to a treatment to CFS if the expression level of the biomarker is at least 9 fold of a reference expression level of the biomarker. In some embodiments, the method comprises identifying or predicting the subject as likely to be responsive to a treatment to CFS if the expression level of the biomarker is at least 10 fold of a reference expression level of the biomarker. The expression level of a biomarker can be determined using any known method in the art, and exemplary methods are described in more detail in Section 5.3 below. In some embodiments, a level is determined to be higher than a reference level if the level is higher (e.g., statistically significantly higher) than the reference level as observed according to a measurement assay.

In certain embodiments, the method comprises using two or more biomarkers selected from the group consisting of IKZF2, IKZF3, ABCE1, BACH2, CD3D and HSPA8. In certain embodiments, the method comprises using three or more biomarkers selected from the group consisting of IKZF2, IKZF3, ABCE1, BACH2, CD3D and HSPA8. In certain embodiments, the method comprises using four or more biomarkers selected from the group consisting of IKZF2, IKZF3, ABCE1, BACH2, CD3D and HSPA8. In certain embodiments, the method comprises using five or more biomarkers selected from the group consisting of IKZF2, IKZF3, ABCE1, BACH2, CD3D and HSPA8. In certain embodiments, the method comprises using all biomarkers selected from the group consisting of IKZF2, IKZF3, ABCE1, BACH2, CD3D and HSPA8. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined. In some embodiments, when multiple biomarkers are used, the level of each biomarker is compared with a reference level of that biomarker, and the method comprises identifying or predicting the subject as being likely to be responsive to a treatment to CFS if the level of each biomarker is higher than the reference level of that biomarker. In other embodiments, when multiple biomarkers are used, a composite score is calculated based on the multiple biomarkers and compared with a reference composite score, and the method comprises identifying or predicting the subject as being likely to be responsive a treatment to CFS if the composite score is higher than the reference composite score. In some embodiments, a composite score is calculated using the Median Z-Score method. In other embodiments, a composite score is calculated using the Single-Sample Gene Set Enrichment (ssGSEA) method.

In another aspect, provided herein is a selective treatment method comprising administering a treatment of CFS to the subject identified or predicted to be likely to be responsive to the treatment according to a method provided herein. Specifically, in some embodiments, provided herein is a method of selectively treating a subject having or suspected of having CFS with a treatment, the method comprising obtaining an expression level of a biomarker in a sample from the subject, wherein the biomarker is a CAP selected from the group consisting of IKZF2, IKZF3, ABCE1, BACH2, CD3D and HSPA8; identifying or predicting the subject as being likely to be responsive to a treatment of CFS if the expression level of the biomarker is higher than a reference expression level of the biomarker; and administering the treatment to the subject identified or predicted to be likely to be responsive to the treatment.

The treatment of CFS in the above mentioned method includes any treatment for reducing any symptom associated with CFS. In some embodiments, the treatment comprises an immunomodulatory compound.

In some specific embodiments, the treatment for CFS comprises an immunomodulatory drug (IMiD). IMiDs comprise a group of compounds that can be useful to treat several types of human diseases, including certain cancers. As used herein and unless otherwise indicated, the term “immunomodulatory compound” can encompass certain small organic molecules that inhibit LPS induced monocyte TNF-α, IL-1B, IL-12, IL-6, MIP-1α, MCP-1, GM-CSF, G-CSF, and COX-2 production. These compounds can be prepared synthetically or can be obtained commercially. The inflammatory cytokine TNF-α, which is produced by macrophages and monocytes during acute inflammation, causes a diverse range of signaling events within cells. Without being limited by a particular theory, one of the biological effects exerted by the immunomodulatory compounds disclosed herein is the reduction of myeloid cell TNF-α production. Immunomodulatory compounds disclosed herein may enhance the degradation of TNF-α mRNA. Further, without being limited by theory, immunomodulatory compounds disclosed herein may also be potent co-stimulators of T cells and increase cell proliferation dramatically in a dose dependent manner. Immunomodulatory compounds disclosed herein may also have a greater co-stimulatory effect on the CD8+ T cell subset than on the CD4+ T cell subset. In addition, the compounds may have anti-inflammatory properties against myeloid cell responses, yet efficiently co-stimulate T cells to produce greater amounts of IL-2, IFN-γ, and to enhance T cell proliferation and CD8+ T cell cytotoxic activity. Further, without being limited by a particular theory, immunomodulatory compounds disclosed herein may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-γ, and to enhance NK and NKT cell cytotoxic activity. Various immunomodulatory compounds disclosed herein contain one or more chiral centers, and can exist as racemic mixtures of enantiomers or mixtures of diastereomers. Thus, also provided herein is the use of stereomerically pure forms of such compounds, as well as the use of mixtures of those forms. For example, mixtures comprising equal or unequal amounts of the enantiomers of a particular immunomodulatory compounds may be used. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

In other embodiments, the treatment of CFS provided herein comprises a CRBN modulator. In some embodiments, a CRBN modulator is an agent that can modulate at least one of CRBN's biological activities directly or indirectly. In some embodiments, a CRBN modulator is an agent that can physically bind to CRBN. In other embodiments, a CRBN modulator does not directly bind to CRBN, but can otherwise exert an effect via a CRBN mediated pathway. Cereblon (CRBN), a component of the DDB1-CUL4a-Roc1 ubiquitin ligase complex, has been identified as a target of certain immunomodulatory compounds, e.g., thalidomide, lenalidomide, pomalidomide, and iberdomide (Lopez-Girona et al., Leukemia volume 26, pages 2326-2335 (2012); Bjorklund et al., Leukemia. 2020; 34(4): 1197-1201). It is believed that the interactions of CRBN with certain immunomodulatory compounds mediate their anti-proliferative effects in multiple myeloma (MM) cells (Lopez-Girona et al., Leukemia 2012; Zhu et al., Blood 2011, 118, Abstract 127). CRBN is encoded by a 25 kb gene on chromosome 6, consisting of 11 exons and 10 introns. Thus, alternative splicing process can potentially generate multiple functional proteins as well as variants of a protein from a single gene having different structural organization and functional activity. Truncated proteins that have lost interaction domains or critical functional amino acid residues may create non-functional or aberrant CRBN proteins that may interfere with the functions of the full-length CRBN protein and reduce or alter the therapeutic activity of a treatment compound that exerts its activity via its interactions with the full-length CRBN protein. At least two isoforms of the protein cereblon (CRBN) exist, which are 442 and 441 amino acids long, respectively, and CRBN is conserved from plant to human. In humans, the CRBN gene has been identified as a candidate gene of an autosomal recessive nonsyndromic mental retardation (ARNSMR). See Higgins, J. J. et al., Neurology, 2004, 63:1927-1931. CRBN was initially characterized as an RGS-containing novel protein that interacted with a calcium-activated potassium channel protein (SLO1) in the rat brain, and was later shown to interact with a voltage-gated chloride channel (CIC-2) in the retina with AMPK1 and DDB1. See Jo, S. et al., J. Neurochem, 2005, 94:1212-1224; Hohberger B. et al., FEBS Lett, 2009, 583:633-637; Angers S. et al., Nature, 2006, 443:590-593. DDB1 was originally identified as a nucleotide excision repair protein that associates with damaged DNA binding protein 2 (DDB2). Its defective activity causes the repair defect in the patients with xeroderma pigmentosum complementation group E (XPE). DDB1 also appears to function as a component of numerous distinct DCX (DDB1-CUL4-X-box) E3 ubiquitin-protein ligase complexes which mediate the ubiquitination and subsequent proteasomal degradation of target proteins. CRBN has also been identified as a target for the development of therapeutic agents for diseases of the cerebral cortex. See WO 2010/137547 A1. In some embodiments, binding to CRBN or one or more substrates of CRBN is required for the beneficial effects of certain treatment compounds provided herein. In some embodiments, the compound provided herein to treat CFS can induce CRBN to undergo conformational changes. In some embodiments, the use of a treatment compound provided herein leads to a distinct conformational change or other alteration in the properties of the CRBN surface, and a resulting distinct phenotypic response. In certain embodiments, a CRBN modulator is an immunomodulatory compound. In other embodiments, a CRBN is not an immunomodulatory compound.

In some embodiments, the treatment of CFS disclosed herein comprises an agent that depletes B cells. In some embodiments, the agent that depletes B cells is an antibody that specifically binds an antigen of B cells. In some embodiments, the antigen of B cells is CD20, CD19, CD22, CD38, or B-cell activating factor (BAFF). In some embodiments, the agent that depletes B cells is an anti-CD20 antibody. In some embodiments, the anti-CD20 antibody is rituximab, ocrelizumab, or ofatumumab. the agent that depletes B cells is an anti-CD19 antibody. In some embodiments, the anti-CD19 antibody is inebilizumab. In some embodiments, the agent that depletes B cells is an anti-BAFF antibody. In some embodiments, the anti-BAFF antibody is belimumab.

In some embodiments, the treatment compound in the present selective treatment method is formulated in a pharmaceutical composition which comprises a treatment compound provided herein and a pharmaceutically acceptable excipient. Pharmaceutical compositions comprising treatment compounds provided herein are prepared for storage by mixing the compound provided herein with optional physiologically acceptable excipients (see, e.g., Remington, Remington's Pharmaceutical Sciences (18^{th} ed. 1980)) in the form of aqueous solutions or lyophilized or other dried forms. The treatment compound of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45). The treatment compound provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra. Various compositions and delivery systems are known and can be used with a compound as described herein. In some embodiments, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising the treatment compound described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60). Various delivery systems are known and can be used to administer a treatment compound provided herein.

In yet another aspect according to the present disclosure, a subject's responsiveness to a treatment compound can be determined by a change of the expression level of a biomarker provided herein. For example, a decrease in the expression level of a biomarker provided herein upon a treatment indicates that the subject is responsive to the treatment; whereas no change of the expression level of the biomarker indicates that the subject is not responsive. The degree of the change of the biomarker may also be used to indicate the degree of the responsiveness. A treatment and dose adjustment can then be designed accordingly. Thus, in some embodiments, provided herein is a method of determining or monitoring effectiveness of a treatment in a subject having CFS comprising obtaining a first expression level of a biomarker in a first sample from the subject, wherein the biomarker is a CAP selected from the group consisting of IKZF2, IKZF3, ABCE1, BACH2, CD3D and HSPA8; administering the treatment to the subject; obtaining a second expression level of the biomarker in a second sample obtained from the subject after administering the treatment to the subject; and determining the effectiveness of the treatment based on the comparison of the first expression level with the second expression level. In some embodiments, the method comprises determining that the treatment is effective if the second expression level is lower than the first expression level. In some embodiments, the method comprises determining that the treatment is not effective if the second expression level is not lower than the first expression level. In some embodiments, the method comprises determining or adjusting (e.g., increasing) the dose of the treatment or administering a different treatment to the subject if the second expression level is not lower than the first expression level

In some embodiments, the method comprises determining that the treatment is effective if the second expression level is at least 10% lower than the first expression level. In some embodiments, the method comprises determining that the treatment is effective if the second expression level is at least 20% lower than the first expression level. In some embodiments, the method comprises determining that the treatment is effective if the second expression level is at least 30% lower than the first expression level. In some embodiments, the method comprises determining that the treatment is effective if the second expression level is at least 40% lower than the first expression level. In some embodiments, the method comprises determining that the treatment is effective if the second expression level is at least 50% lower than the first expression level. In some embodiments, the method comprises determining that the treatment is effective if the second expression level is at least 60% lower than the first expression level. In some embodiments, the method comprises determining that the treatment is effective if the second expression level is at least 70% lower than the first expression level. In some embodiments, the method comprises determining that the treatment is effective if the second expression level is at least 80% lower than the first expression level. In some embodiments, the method comprises determining that the treatment is effective if the second expression level is at least 90% lower than the first expression level. In some embodiments, the treatment comprises an immunomodulatory drug (IMiD). In other embodiments, the treatment comprises a CRBN modulator. In some embodiments, the treatment comprises an agent that depletes B cells (e.g., an anti-CD20 antibody, e.g., rituximab).

Based on the comparison of the first expression level and the second expression level, a different treatment or a different dosing regimen may be administered to the subject in the subsequent treatment cycle(s). In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8. The expression level of a biomarker can be determined using any known method in the art, and exemplary methods are described in more detail in Section 5.3 below. In some embodiments, a level is determined to be higher than a second level if the level is higher (e.g., statistically significantly higher) than the second level as observed according to a measurement assay. In some embodiments, a level is determined to be lower than a second level if the level is lower (e.g., statistically significant) than the second level as observed according to a measurement assay.

In certain embodiments, the method comprises using two or more biomarkers selected from the group consisting of IKZF2, IKZF3, ABCE1, BACH2, CD3D and HSPA8. In certain embodiments, the method comprises using three or more biomarkers selected from the group consisting of IKZF2, IKZF3, ABCE1, BACH2, CD3D and HSPA8. In certain embodiments, the method comprises using four or more biomarkers selected from the group consisting of IKZF2, IKZF3, ABCE1, BACH2, CD3D and HSPA8. In certain embodiments, the method comprises using five or more biomarkers selected from the group consisting of IKZF2, IKZF3, ABCE1, BACH2, CD3D and HSPA8. In certain embodiments, the method comprises using all biomarkers selected from the group consisting of IKZF2, IKZF3, ABCE1, BACH2, CD3D and HSPA8. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined. In some embodiments, when multiple biomarkers are used, the second expression level of each biomarker is compared with a first expression level of that biomarker, and the method comprises determining that the treatment is effective if the second expression level of each biomarker is lower than the first expression level of that biomarker. In other embodiments, when multiple biomarkers are used, a composite score is calculated based on the multiple biomarkers and compared with a reference composite score. In some embodiments, a composite score is calculated using the Median Z-Score method. In other embodiments, a composite score is calculated using the Single-Sample Gene Set Enrichment (ssGSEA) method.

The present disclosure also includes uses of the biomarkers provided herein to screen compounds for effectiveness in treating CFS. Thus, in some embodiments, provided herein is a method of screening a compound for effectiveness in treating CFS comprising obtaining a first expression level of a biomarker in a sample, wherein the biomarker is a CAP selected from the group consisting of IKZF2, IKZF3, ABCE1, BACH2, CD3D and HSPA8; administering the compound to the sample; obtaining a second expression level of the biomarker in the sample after administering the compound to the sample; comparing the first expression level with the second expression level; and selecting the compound if the second expression level is lower than the first expression level. In some embodiments, the method comprises selecting the compound if the second expression level is at least 10% lower than the first expression level. In some embodiments, the method comprises selecting the compound if the second expression level is at least 20% lower than the first expression level. In some embodiments, the method comprises selecting the compound if the second expression level is at least 30% lower than the first expression level. In some embodiments, the method comprises selecting the compound if the second expression level is at least 40% lower than the first expression level. In some embodiments, the method comprises selecting the compound if the second expression level is at least 50% lower than the first expression level. In some embodiments, the method comprises selecting the compound if the second expression level is at least 60% lower than the first expression level. In some embodiments, the method comprises selecting the compound if the second expression level is at least 70% lower than the first expression level. In some embodiments, the method comprises selecting the compound if the second expression level is at least 80% lower than the first expression level. In some embodiments, the method comprises selecting the compound if the second expression level is at least 10% lower than the first expression level. In some embodiments, the method comprises selecting the compound if the second expression level is at least 90% lower than the first expression level. In some embodiments, the treatment compound is an immunomodulatory drug (IMiD). In other embodiments, the treatment compound is a CRBN modulator or a compound capable of binding and/or inducing conformational change to CRBN. In some embodiments, the treatment compound is an agent that depletes B cells (e.g., an anti-CD20 antibody, e.g., rituximab). In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8. In certain embodiments, the method comprises using two or more biomarkers selected from the group consisting of IKZF2, IKZF3, ABCE1, BACH2, CD3D and HSPA8. In certain embodiments, the method comprises using three or more biomarkers selected from the group consisting of IKZF2, IKZF3, ABCE1, BACH2, CD3D and HSPA8. In certain embodiments, the method comprises using four or more biomarkers selected from the group consisting of IKZF2, IKZF3, ABCE1, BACH2, CD3D and HSPA8. In certain embodiments, the method comprises using five or more biomarkers selected from the group consisting of IKZF2, IKZF3, ABCE1, BACH2, CD3D and HSPA8. In certain embodiments, the method comprises using all biomarkers selected from the group consisting of IKZF2, IKZF3, ABCE1, BACH2, CD3D and HSPA8. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined. In some embodiments, when multiple biomarkers are used, the second expression level of each biomarker is compared with a first expression level of that biomarker, and the method comprises selecting the compound if the second expression level of each biomarker is lower than the first expression level of that biomarker. In other embodiments, when multiple biomarkers are used, a composite score is calculated based on the multiple biomarkers and compared with a reference composite score, and selecting the compound if the composite score is higher than the reference composite score. In some embodiments, the method comprises obtaining a first composite score based on the first expression levels of the biomarkers and a second composite score based on the second expression level of the biomarkers, and comparing the first composite score with the second composite score. In some embodiments, a composite score is calculated using the Median Z-Score method. In other embodiments, a composite score is calculated using the Single-Sample Gene Set Enrichment (ssGSEA) method.

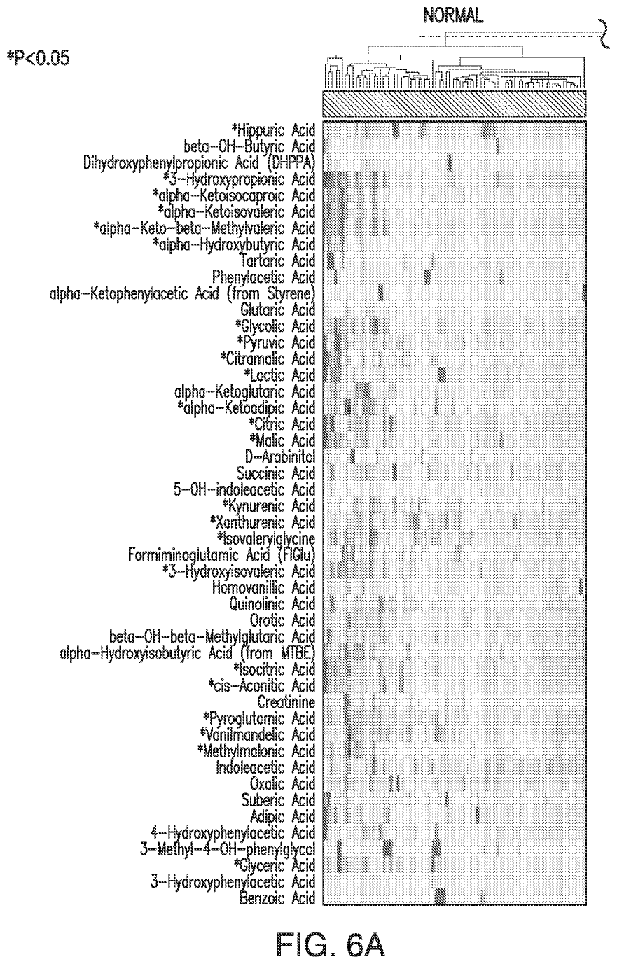

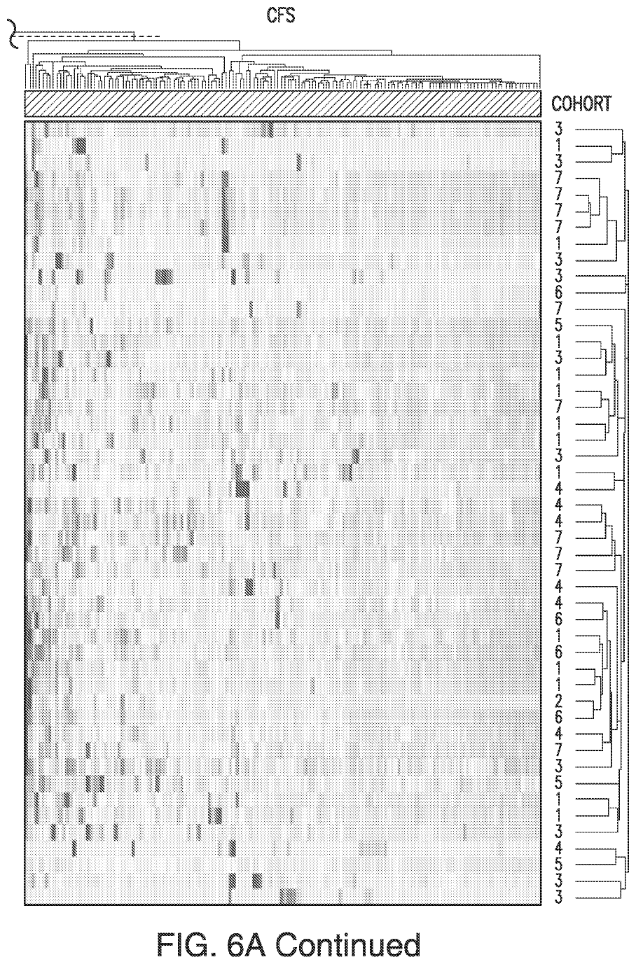

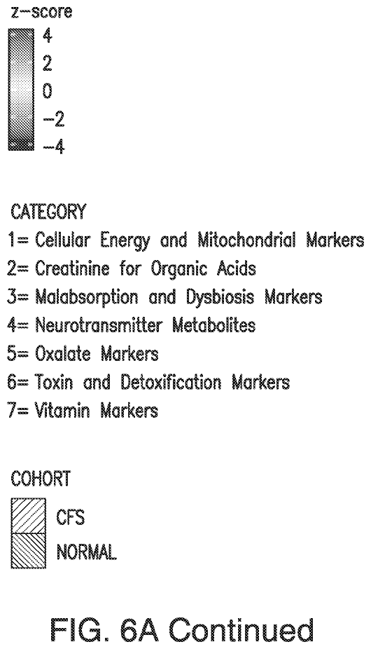

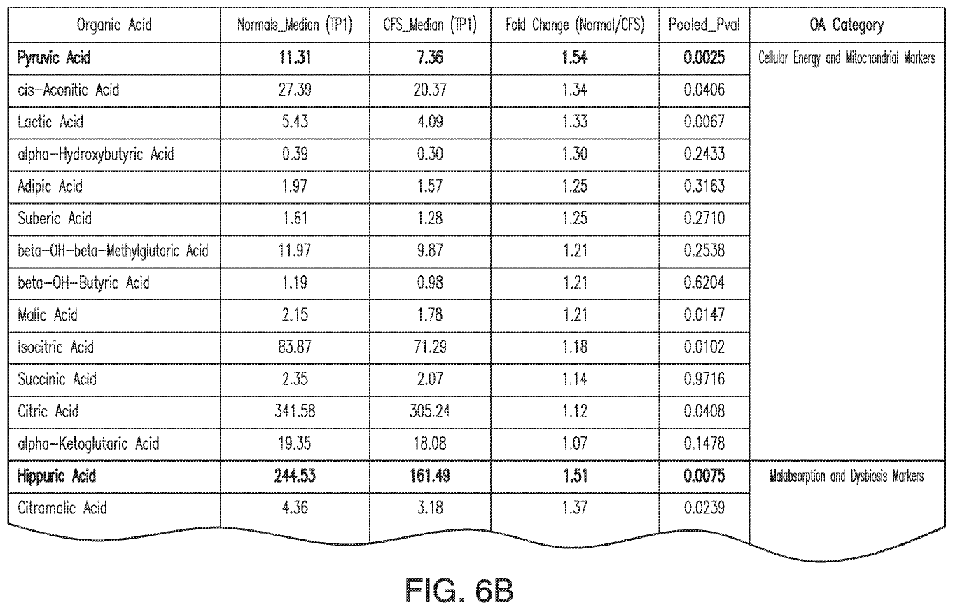

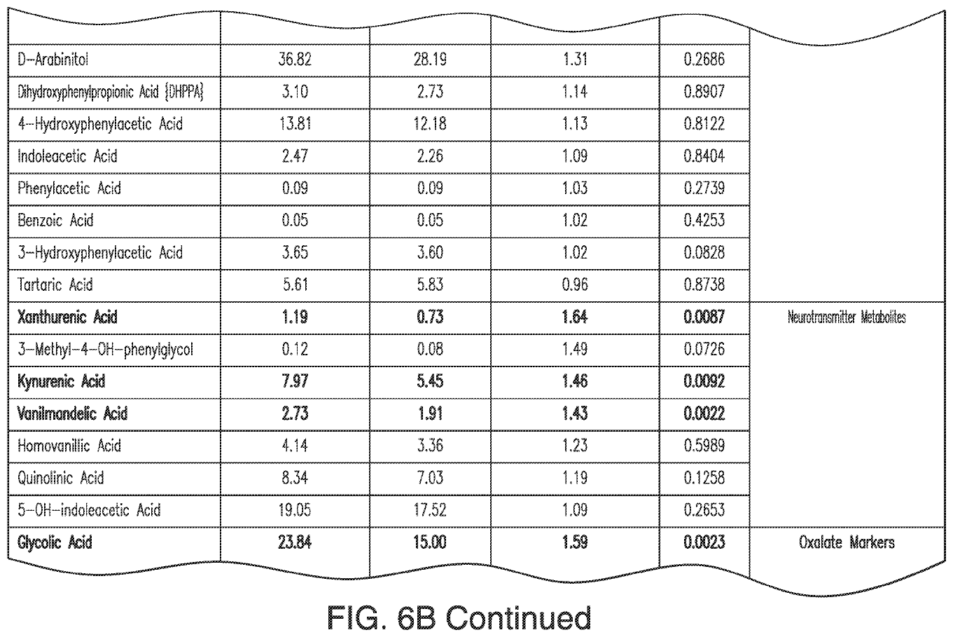

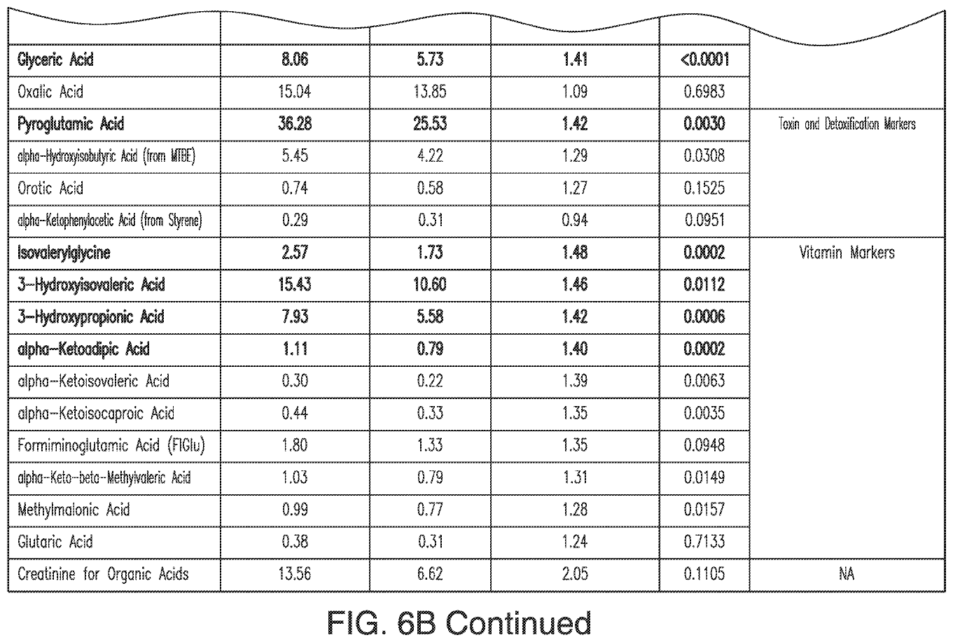

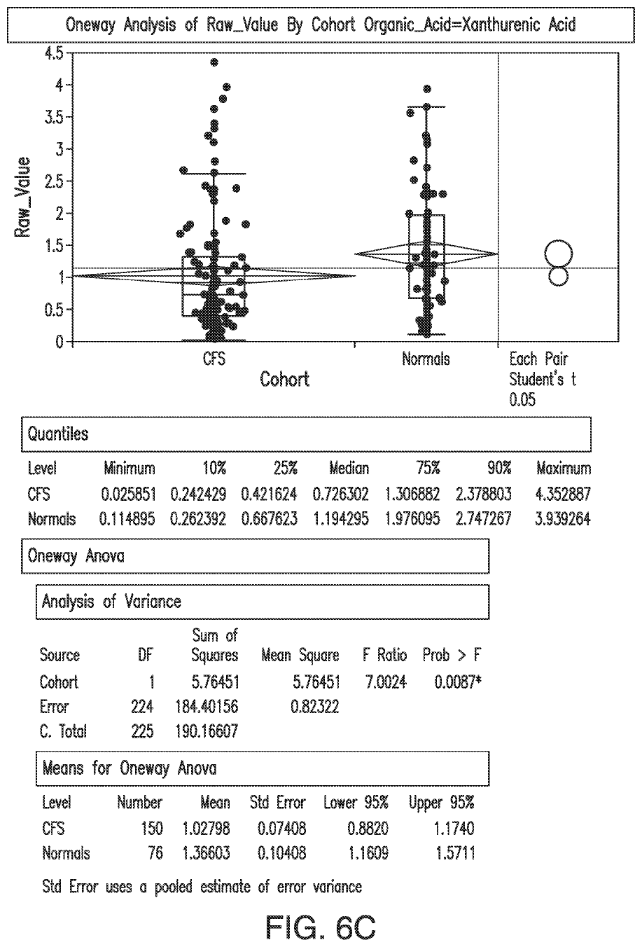

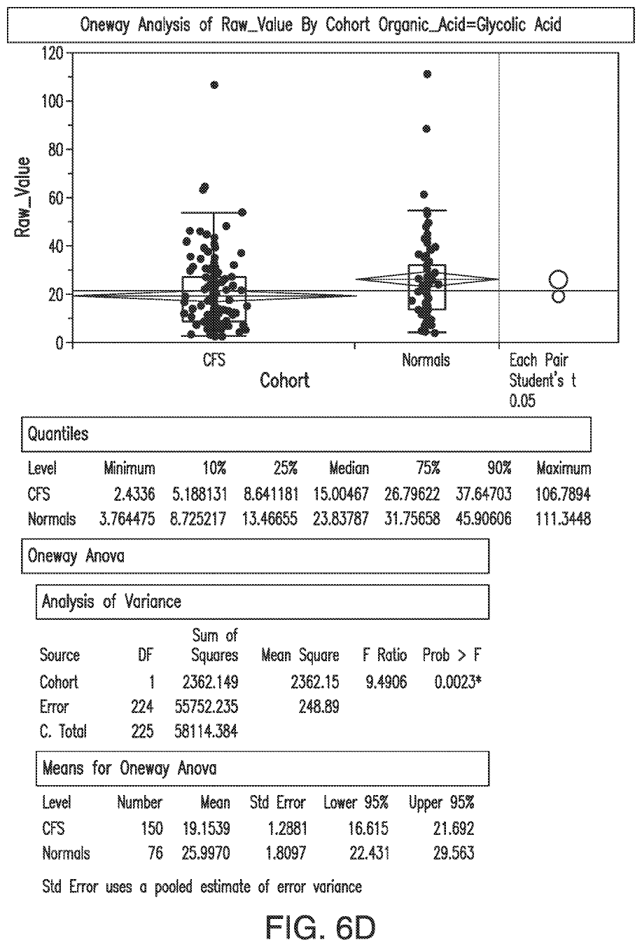

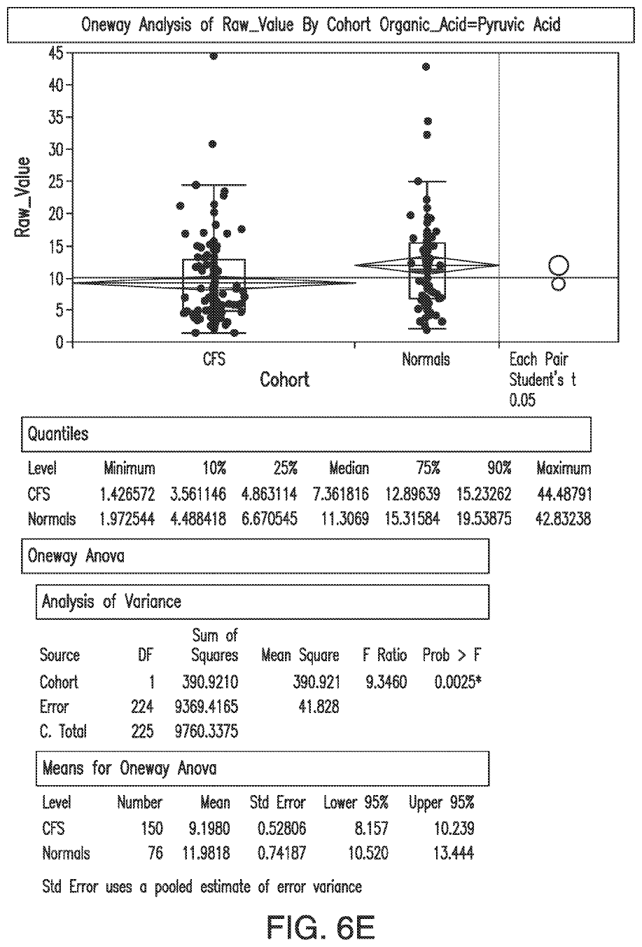

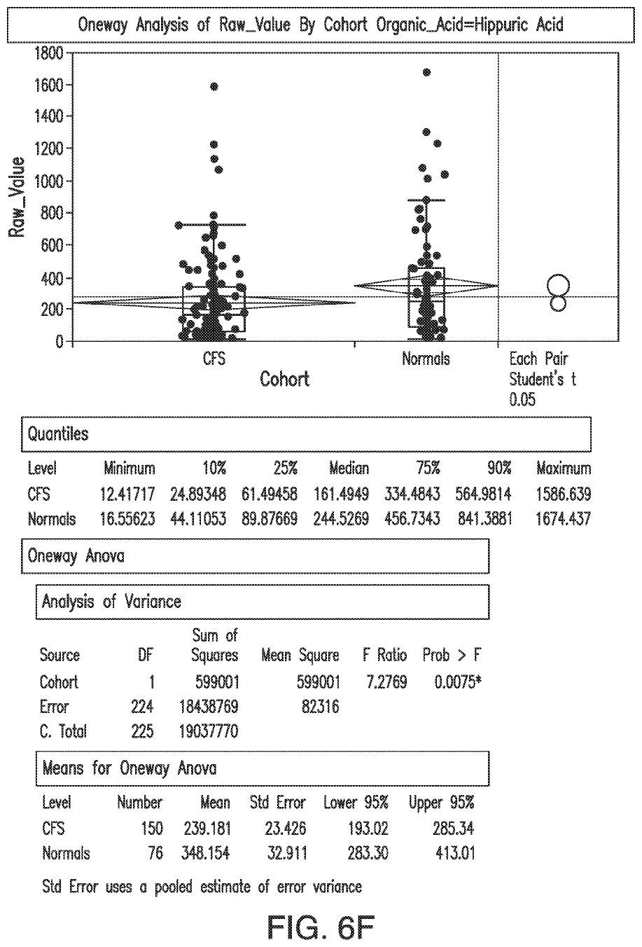

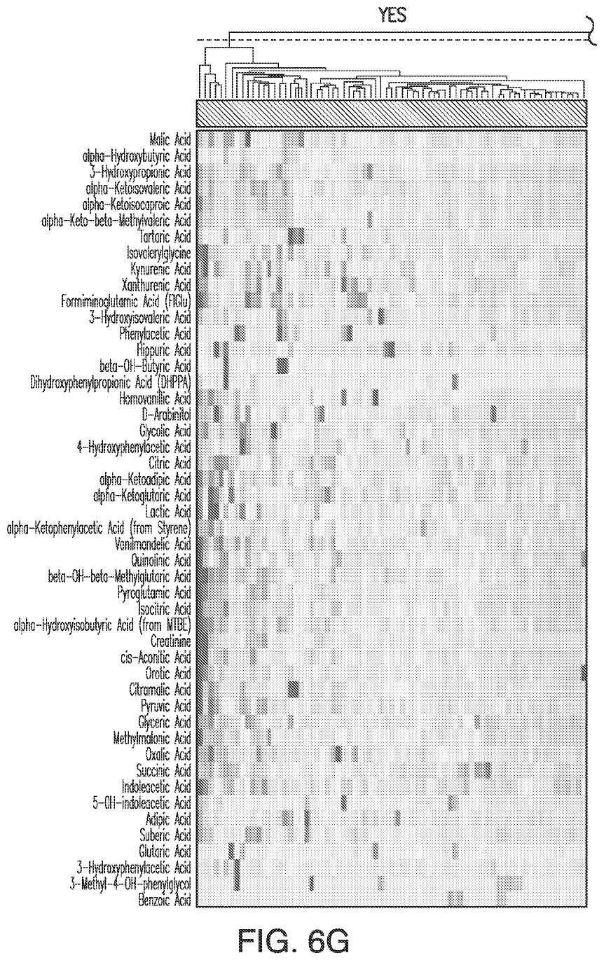

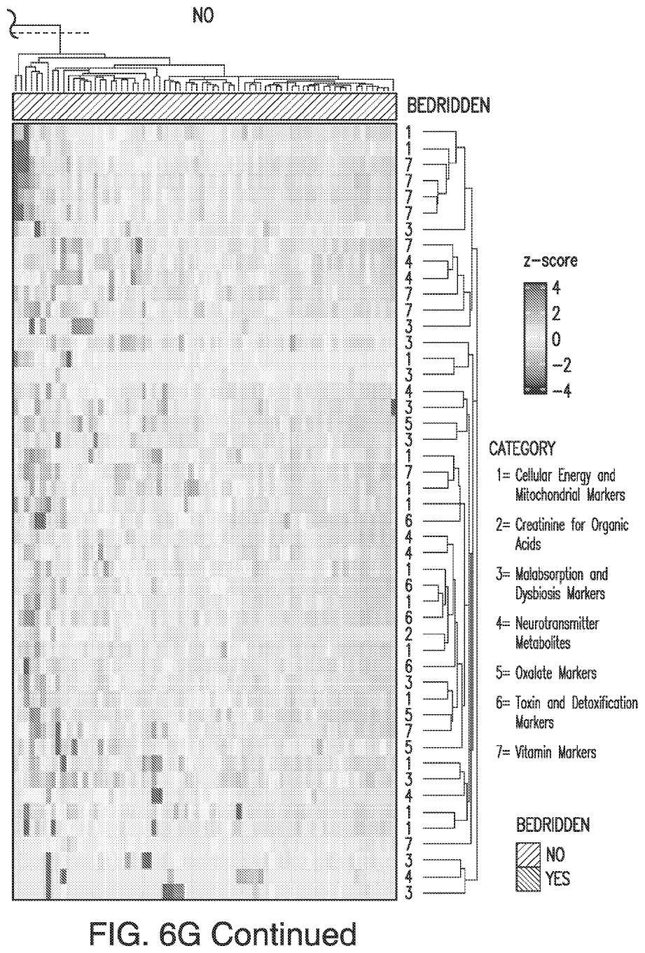

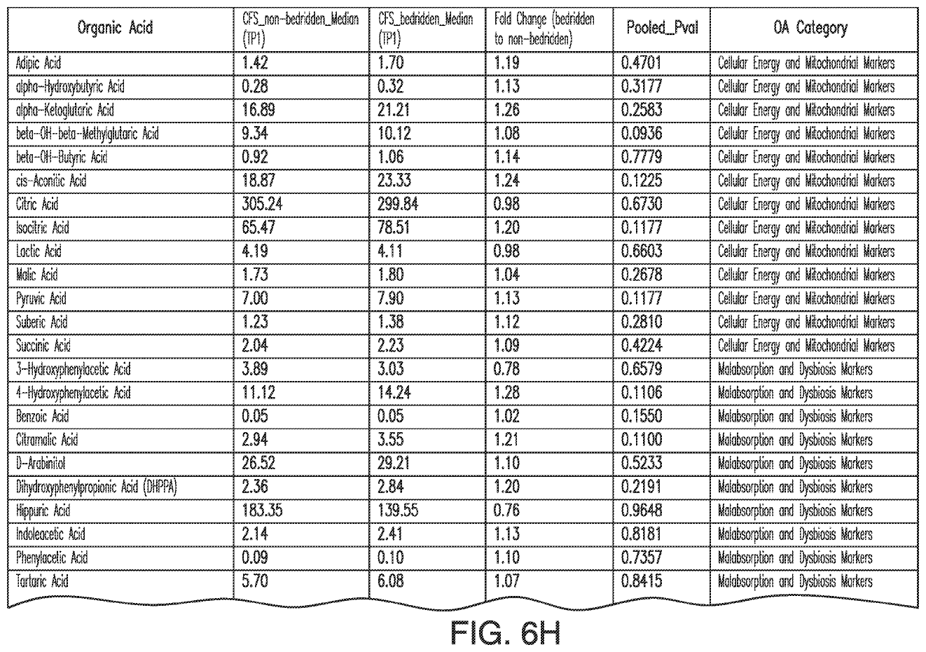

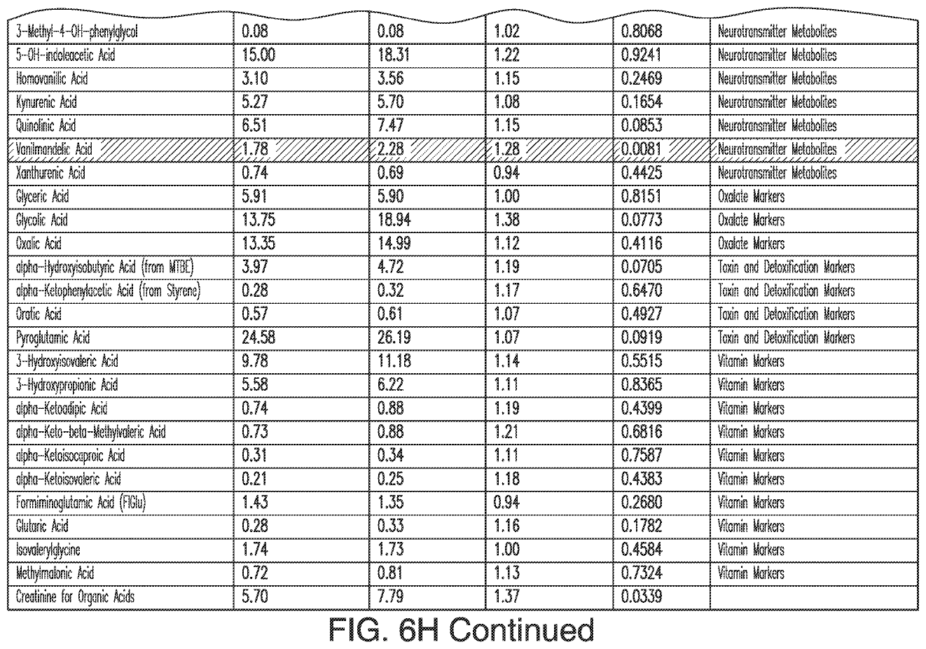

In another aspect, the present disclosure also provides methods and kits based on the surprising finding of the correlation between CSF and certain organic acids. As shown in the Example section, urine organic acid profiling reveals difference between CFS and normal cohorts. More specifically, profiles of urinary organic acids showed statistically significant (p<0.05) differences between CFS patients and normal subjects in 23 organic acids-hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, glyceric acid (see FIGS. 6A-6F). Among them, 12 organic acids had median fold change of >1.4 (decreased in CFS) and p<0.05 and they are xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In addition, there was significant difference in vanilmandelic acid level between CFS bedridden and non-bedridden subjects. Thus, one or more of these organic acids can be used in various methods described above in combination with IKZF2, IKZF3, ABCE1, BACH2, CD3D and/or HSPA8. In other embodiments, one or more of these organic acids can be used independent of IKZF2, IKZF3, ABCE1, BACH2, CD3D and/or HSPA8 in various methods, e.g., for identifying a subject having CFS or verifying CFS in a subject, determining severity of CFS in a subject, identifying a subject who is likely or not likely to be responsive to a treatment of CFS or predicting the responsiveness of a subject to a treatment of CFS, selectively treating a subject having or suspected of having CFS with a treatment, and/or determining or monitoring effectiveness of a treatment in a subject having CFS as well as screening a compound for effectiveness in treating CFS, as described in Embodiments 56-94 below.

In some embodiments, provided herein is a method of identifying a subject having Chronic Fatigue Syndrome (CFS) or verifying CFS in a subject, comprising obtaining a level of a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; and identifying or verifying the subject as having CFS if the level of the urine organic acid in the sample is different from a reference level of the urine organic acid. In some embodiments, the urine organic acid is selected from the group consisting of xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In some embodiments, the reference level of the organic acid is a predetermined level of the organic acid obtained from a public database. In some embodiments, the reference level of the organic acid is a level of the organic acid in a healthy subject or a subject who does not have CFS. In some embodiments, the reference level of the organic acid is a level of the organic acid in a subject having moderate CFS. In some embodiments, the reference level of the organic acid is a level of the organic acid determined based on a cohort of subject (e.g. a cohort of healthy subjects, a cohort of subject not having CFS, or a cohort of subject having moderate CFS). In some embodiments, the reference level of the organic acid is a median or a mean level of the organic acid of the levels of the organic acid in a cohort of subjects (e.g., a cohort of healthy subjects, a cohort of subjects not having CFS, or a cohort of subjects having moderate CFS). In some embodiments, the method comprises identifying or verifying the subject as having CFS if the level of the urine organic acid is lower than a reference level of the urine organic acid. In some embodiments, the method comprises identifying the subject as having CFS if the level of the organic acid is at least 5% lower than a reference level of the organic acid. In some embodiments, the method comprises identifying the subject as having CFS if the level of the organic acid is at least 10% lower than a reference level of the organic acid. In some embodiments, the method comprises identifying the subject as having CFS if the level of the organic acid is at least 20% lower than a reference level of the organic acid. In some embodiments, the method comprises identifying the subject as having CFS if the level of the organic acid is at least 30% lower than a reference level of the organic acid. In some embodiments, the method comprises identifying the subject as having CFS if the level of the organic acid is at least 40% lower than a reference level of the organic acid. In some embodiments, the method comprises identifying the subject as having CFS if the level of the organic acid is at least 50% lower than a reference level of the organic acid. In some embodiments, the method comprises identifying the subject as having CFS if the level of the organic acid is at least 60% lower than a reference level of the organic acid. In some embodiments, the method comprises identifying the subject as having CFS if the level of the organic acid is at least 70% lower than a reference level of the organic acid. In some embodiments, the method comprises identifying the subject as having CFS if the level of the organic acid is at least 80% lower than a reference level of the organic acid. In some embodiments, the method comprises identifying the subject as having CFS if the level of the organic acid is at least 90% lower than a reference level of the organic acid. In some embodiments, the method comprises identifying the subject as having CFS if the level of the organic acid is at least 2 fold lower compared to a reference level of the organic acid. In some embodiments, the method comprises identifying the subject as having CFS if the level of the organic acid is at least 3 fold lower compared to a reference level of the organic acid. In some embodiments, the method comprises identifying the subject as having CFS if the level of the organic acid is at least 4 fold lower compared to a reference level of the organic acid. In some embodiments, the method comprises identifying the subject as having CFS if the level of the organic acid is at least 5 fold lower compared to a reference level of the organic acid. In some embodiments, the method comprises identifying the subject as having CFS if the level of the organic acid is at least 6 fold lower compared to a reference level of the organic acid. In some embodiments, the method comprises identifying the subject as having CFS if the level of the organic acid is at least 7 fold lower compared to a reference level of the organic acid. In some embodiments, the method comprises identifying the subject as having CFS if the level of the organic acid is at least 8 fold lower compared to a reference level of the organic acid. In some embodiments, the method comprises identifying the subject as having CFS if the level of the organic acid is at least 9 fold lower compared to a reference level of the organic acid. In some embodiments, the method comprises identifying the subject as having CFS if the level of the organic acid is at least 10 fold lower compared to a reference level of the organic acid. The level of an organic acid can be determined using any known method in the art, and exemplary methods are described in more detail in Section 5.3 below. In some embodiments, a level of the organic acid is determined to be lower than a reference level if the level is lower (e.g., statistically significant) than the reference level as observed according to a measurement assay. In certain embodiments, the method comprises using two or more organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid to identify a subject having CFS. In some embodiments, the method comprises identifying or verifying the subject as having CFS if the level of the urine organic acid in the sample is lower than a reference level of the urine organic acid. In some embodiments, the CFS is associated with an autoimmune disease or an infection.

In some embodiments, provided herein is a method of determining severity of CFS in a subject, comprising obtaining a level of a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; comparing the level of the urine organic acid in the sample with a reference level of the urine organic acid; and determining the severity of CFS in the subject based on the comparison of the level of the urine organic in the sample to the reference level of the urine organic acid. In some embodiments, the urine organic acid is selected from the group consisting of xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In some embodiments, the method further comprises comparing the level of the organic acid with a reference level of the organic acid. In some embodiments, a lower level of the organic acid indicates more severe CFS. In yet other embodiments, provided herein is a method of monitoring progress of CFS in a subject comprising obtaining a level of an organic acid in a sample from the subject, wherein the organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid, and assessing the progress of CFS based on the level of the biomarker In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In certain embodiments, the method comprises using two or more organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid to identify a subject having CFS. In some embodiments, the reference level of the organic acid is a predetermined level of the organic acid obtained from a public database. In some embodiments, the reference level of the organic acid is a level of the organic acid in a healthy subject or a subject who does not have CFS. In some embodiments, the reference level of the organic acid is a level of the organic acid in a subject having moderate CFS. In some embodiments, the reference level of the organic acid is a level of the organic acid determined based on a cohort of healthy subjects. In some embodiments, the reference level of the organic acid is a level of the organic acid in a subject whose severity of CFS has been determined and known. In some embodiments, the reference level of the organic acid is a median or a mean level of the organic acid of the levels of the organic acid in a cohort of subjects (e.g., a cohort of healthy subjects, a cohort of subjects having moderate CFS, or a cohort of subjects whose severity of CFS has been determined and known). In other embodiments, the reference level of the organic acid is a level of the organic acid in the same subject but measured at a different time. The level of an organic acid can be determined using any known method in the art, and exemplary methods are described in more detail in Section 5.3 below. In some embodiments, a level is determined to be higher than a reference level if the level is higher (e.g., statistically significantly higher) than the reference level as observed according to a measurement assay. In some embodiments, a level is determined to be lower than a reference level if the level is lower (e.g., statistically significant) than the reference level as observed according to a measurement assay. In certain embodiments, the method comprises using two or more organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid to identify a subject having CFS. In some embodiments, the CFS is associated with an autoimmune disease or an infection.

In some embodiments, provided herein is a method of identifying a subject who is likely or not likely to be responsive to a treatment of CFS or predicting the responsiveness of a subject to a treatment of CFS, comprising obtaining a level a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; and identifying or predicting the subject as being likely to be responsive to a treatment of CFS if the level of the urine organic acid in the sample is different from a reference level of the urine organic acid. In some embodiments, the urine organic acid is selected from the group consisting of xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In some embodiments, the reference level of the organic acid is a predetermined level of the organic acid obtained from a public database. In some embodiments, the reference level of the organic acid is a level of the organic acid in a healthy subject or a subject who does not have CFS. In some embodiments, the reference level of the organic acid is a level of the organic acid in a subject having moderate CFS. In some embodiments, the reference level of the organic acid is a level of the organic acid determined based on a cohort of subjects (e.g., a cohort of healthy subjects, a cohort of subjects not having CFS, or a cohort of subjects having moderate CFS). In some embodiments, the reference level of the organic acid is a median or a mean level of the organic acid of the levels of the organic acid in a cohort of subjects (e.g., a cohort of healthy subjects, a cohort of subjects not having CFS, or a cohort of subjects having moderate CFS). In some embodiments, the method comprises identifying or predicting the subject as likely to be responsive to a treatment of CFS if the level of the organic acid is lower than a reference level of the organic acid. In some embodiments, the method comprises identifying or predicting the subject as likely to be responsive to a treatment of CFS if the level of the organic acid is at least 5% lower than a reference level of the organic acid. In some embodiments, the method comprises identifying or predicting the subject as likely to be responsive to a treatment of CFS if the level of the organic acid is at least 10% lower than a reference level of the organic acid. In some embodiments, the method comprises identifying or predicting the subject as likely to be responsive to a treatment of CFS if the level of the organic acid is at least 20% lower than a reference level of the organic acid. In some embodiments, the method comprises identifying or predicting the subject as likely to be responsive to a treatment of CFS if the level of the organic acid is at least 30% lower than a reference level of the organic acid. In some embodiments, the method comprises identifying or predicting the subject as likely to be responsive to a treatment of CFS if the level of the organic acid is at least 40% lower than a reference level of the organic acid. In some embodiments, the method comprises identifying or predicting the subject as likely to be responsive to a treatment of CFS if the level of the organic acid is at least 50% lower than a reference level of the organic acid. In some embodiments, the method comprises identifying or predicting the subject as likely to be responsive to a treatment of CFS if the level of the organic acid is at least 60% lower than a reference level of the organic acid. In some embodiments, the method comprises identifying or predicting the subject as likely to be responsive to a treatment of CFS if the level of the organic acid is at least 70% lower than a reference level of the organic acid. In some embodiments, the method comprises identifying or predicting the subject as likely to be responsive to a treatment of CFS if the level of the organic acid is at least 80% lower than a reference level of the organic acid. In some embodiments, the method comprises identifying or predicting the subject as likely to be responsive to a treatment of CFS if the level of the organic acid is at least 90% lower than a reference level of the organic acid. In some embodiments, the method comprises identifying or predicting the subject as likely to be responsive to a treatment of CFS if the level of the organic acid is at least 2 fold lower compared to a reference level of the organic acid. In some embodiments, the method comprises identifying or predicting the subject as likely to be responsive to a treatment of CFS if the level of the organic acid is at least 3 fold lower compared to a reference level of the organic acid. In some embodiments, the method comprises identifying or predicting the subject as likely to be responsive to a treatment of CFS if the level of the organic acid is at least 4 fold lower compared to a reference level of the organic acid. In some embodiments, the method comprises identifying or predicting the subject as likely to be responsive to a treatment of CFS if the level of the organic acid is at least 5 fold lower compared to a reference level of the organic acid. In some embodiments, the method comprises identifying or predicting the subject as likely to be responsive to a treatment of CFS if the level of the organic acid is at least 6 fold lower compared to a reference level of the organic acid. In some embodiments, the method comprises identifying or predicting the subject as likely to be responsive to a treatment of CFS if the level of the organic acid is at least 7 fold lower compared to a reference level of the organic acid. In some embodiments, the method comprises identifying or predicting the subject as likely to be responsive to a treatment of CFS if the level of the organic acid is at least 8 fold lower compared to a reference level of the organic acid. In some embodiments, the method comprises identifying or predicting the subject as likely to be responsive to a treatment of CFS if the level of the organic acid is at least 9 fold lower compared to a reference level of the organic acid. In some embodiments, the method comprises identifying or predicting the subject as likely to be responsive to a treatment of CFS if the level of the organic acid is at least 10 fold lower compared to a reference level of the organic acid. In some embodiments, the method comprises identifying or predicting the subject as being likely to be responsive to a treatment of CFS if the level of the urine organic acid is lower than a reference level of the urine organic acid. In some embodiments, the method comprises administering the treatment to the subject identified or predicted to be likely to be responsive to the treatment. In certain embodiments, the method comprises using two or more organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid to identify a subject having CFS. In some embodiments, the CFS is associated with an autoimmune disease or an infection.

In some embodiments, provided herein is a method of selectively treating a subject having or suspected of having CFS with a treatment, comprising obtaining a level a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; identifying or predicting the subject as being likely to be responsive to a treatment of CFS if the level of the urine organic acid in the sample is different from a reference level of the urine organic acid; and administering the treatment to the subject identified or predicted to be likely to be responsive to the treatment. In some embodiments, the urine organic acid is selected from the group consisting of xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In some embodiments, the reference level of the organic acid is a predetermined level of the organic acid obtained from a public database. In some embodiments, the reference level of the organic acid is a level of the organic acid in a healthy subject or a subject who does not have CFS. In some embodiments, the reference level of the organic acid is a level of the organic acid in a subject having moderate CFS. In some embodiments, the reference level of the organic acid is a level of the organic acid determined based on a cohort of subjects (e.g., a cohort of healthy subjects, a cohort of subjects not having CFS, or a cohort of subjects having moderate CFS). In some embodiments, the reference level of the organic acid is a median or a mean level of the organic acid of the levels of the organic acid in a cohort of subjects (e.g., a cohort of healthy subjects, a cohort of subjects not having CFS, or a cohort of subjects having moderate CFS). In some embodiments, the method comprises identifying or predicting the subject as likely to be responsive to a treatment of CFS if the level of the organic acid is lower than a reference level of the organic acid. In some embodiments, the method comprises identifying or predicting the subject as likely to be responsive to a treatment of CFS if the level of the organic acid is at least 5% lower than a reference level of the organic acid. In some embodiments, the method comprises identifying or predicting the subject as likely to be responsive to a treatment of CFS if the level of the organic acid is at least 10% lower than a reference level of the organic acid. In some embodiments, the method comprises identifying or predicting the subject as likely to be responsive to a treatment of CFS if the level of the organic acid is at least 20% lower than a reference level of the organic acid. In some embodiments, the method comprises identifying or predicting the subject as likely to be responsive to a treatment of CFS if the level of the organic acid is at least 30% lower than a reference level of the organic acid. In some embodiments, the method comprises identifying or predicting the subject as likely to be responsive to a treatment of CFS if the level of the organic acid is at least 40% lower than a reference level of the organic acid. In some embodiments, the method comprises identifying or predicting the subject as likely to be responsive to a treatment of CFS if the level of the organic acid is at least 50% lower than a reference level of the organic acid. In some embodiments, the method comprises identifying or predicting the subject as likely to be responsive to a treatment of CFS if the level of the organic acid is at least 60% lower than a reference level of the organic acid. In some embodiments, the method comprises identifying or predicting the subject as likely to be responsive to a treatment of CFS if the level of the organic acid is at least 70% lower than a reference level of the organic acid. In some embodiments, the method comprises identifying or predicting the subject as likely to be responsive to a treatment of CFS if the level of the organic acid is at least 80% lower than a reference level of the organic acid. In some embodiments, the method comprises identifying or predicting the subject as likely to be responsive to a treatment of CFS if the level of the organic acid is at least 90% lower than a reference level of the organic acid. In some embodiments, the method comprises identifying or predicting the subject as likely to be responsive to a treatment of CFS if the level of the organic acid is at least 2 fold lower compared to a reference level of the organic acid. In some embodiments, the method comprises identifying or predicting the subject as likely to be responsive to a treatment of CFS if the level of the organic acid is at least 3 fold lower compared to a reference level of the organic acid. In some embodiments, the method comprises identifying or predicting the subject as likely to be responsive to a treatment of CFS if the level of the organic acid is at least 4 fold lower compared to a reference level of the organic acid. In some embodiments, the method comprises identifying or predicting the subject as likely to be responsive to a treatment of CFS if the level of the organic acid is at least 5 fold lower compared to a reference level of the organic acid. In some embodiments, the method comprises identifying or predicting the subject as likely to be responsive to a treatment of CFS if the level of the organic acid is at least 6 fold lower compared to a reference level of the organic acid. In some embodiments, the method comprises identifying or predicting the subject as likely to be responsive to a treatment of CFS if the level of the organic acid is at least 7 fold lower compared to a reference level of the organic acid. In some embodiments, the method comprises identifying or predicting the subject as likely to be responsive to a treatment of CFS if the level of the organic acid is at least 8 fold lower compared to a reference level of the organic acid. In some embodiments, the method comprises identifying or predicting the subject as likely to be responsive to a treatment of CFS if the level of the organic acid is at least 9 fold lower compared to a reference level of the organic acid. In some embodiments, the method comprises identifying or predicting the subject as likely to be responsive to a treatment of CFS if the level of the organic acid is at least 10 fold lower compared to a reference level of the organic acid. In certain embodiments, the method comprises using two or more organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid to identify a subject having CFS. In some embodiments, the CFS is associated with an autoimmune disease or an infection.

In some embodiments, provided herein is a method of determining or monitoring effectiveness of a treatment in a subject having CFS comprising obtaining a first level of a urine organic acid in a first sample from the subject before administering the treatment to the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; administering the treatment to the subject, and obtaining a second level of the urine organic acid in a second sample obtained from the subject after administering the treatment to the subject; and determining the effectiveness of the treatment based on the comparison of the first level with the second level. In some embodiments, the urine organic acid is selected from the group consisting of xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In some embodiments, the urine organic acid is selected from the group consisting of xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid. In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In some embodiments, the method comprises determining that the treatment is effective if the second level is higher than the first level. In some embodiments, the method comprises determining that the treatment is effective if the second level is at least 10% higher than the first level. In some embodiments, the method comprises determining that the treatment is effective if the second level is at least 20% higher than the first level. In some embodiments, the method comprises determining that the treatment is effective if the second level is at least 30% higher than the first level. In some embodiments, the method comprises determining that the treatment is effective if the second level is at least 40% higher than the first level. In some embodiments, the method comprises determining that the treatment is effective if the second level is at least 50% higher than the first level. In some embodiments, the method comprises determining that the treatment is effective if the second level is at least 60% higher than the first level. In some embodiments, the method comprises determining that the treatment is effective if the second level is at least 70% higher than the first level. In some embodiments, the method comprises determining that the treatment is effective if the second level is at least 80% higher than the first level. In some embodiments, the method comprises determining that the treatment is effective if the second level is at least 90% higher than the first level. In some embodiments, the method comprises determining or adjusting a dose of the treatment to the subject. In some embodiments, the method comprises determining the levels of two or more urine organic acids and comparing the level of each of the urine organic acids with their respective reference level. In some embodiments, the reference level of the organic acid is a predetermined level of the organic acid from a public database. In some embodiments, the reference level is a level of the organic acid in a healthy subject or a subject who does not have CFS. In some embodiments, the reference level of the organic acid is a level of the organic acid in a subject having moderate CFS. In some embodiments, the reference level of the organic acid is a level of the organic acid determined based on a cohort of healthy subjects. In some embodiments, the reference level of the organic acid is a level of the organic acid in the first sample obtained from the subject. In some embodiments, the reference level of the organic acid is a median or a mean level of the organic acid of the levels of the organic acid in a cohort of subjects (e.g., a cohort of healthy subjects, a cohort of subjects having moderate CFS, or a cohort of first samples obtained from subjects. In some embodiments, the CFS is associated with an autoimmune disease or an infection.

In some embodiments, provided herein is a method of screening a compound for effectiveness in treating CFS, the method comprising obtaining a first level of a urine organic acid in a sample, wherein (i) the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; (ii) xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid; and/or (iii) the urine organic acid is vanilmandelic acid; administering the compound to the sample; obtaining a second level of the urine organic acid in the sample after administering the compound to the sample; comparing the first level with the second level; and selecting the compound if the second level is different from the first level, wherein the compound is selected if the second level is higher than the first level. In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In some embodiments, the method comprises selecting the compound if the second level is higher than the first level. In some embodiments, the method comprises selecting the compound if the second level is 10% higher than the first level. In some embodiments, the method comprises selecting the compound if the second level is 20% higher than the first level. In some embodiments, the method comprises selecting the compound if the second level is 30% higher than the first level. In some embodiments, the method comprises selecting the compound if the second level is 40% higher than the first level. In some embodiments, the method comprises selecting the compound if the second level is 50% higher than the first level. In some embodiments, the method comprises selecting the compound if the second level is 60% higher than the first level. In some embodiments, the method comprises selecting the compound if the second level is 70% higher than the first level. In some embodiments, the method comprises selecting the compound if the second level is 80% higher than the first level. In some embodiments, the method comprises selecting the compound if the second level is 90% higher than the first level. In some embodiments, the method comprises determining the levels of two or more urine organic acids and comparing the level of each of the urine organic acids with their respective reference level. In some embodiments, the reference level of the organic acid is a predetermined level of the organic acid from a public database. In some embodiments, the reference level is a level of the organic acid in a healthy subject or a subject who does not have CFS. In some embodiments, the reference level of the organic acid is a level of the organic acid in a subject having moderate CFS. In some embodiments, the reference level of the organic acid is a level of the organic acid determined based on a cohort of healthy subjects. In some embodiments, the reference level of the organic acid is a level of the organic acid in the first sample obtained from the subject. In some embodiments, the reference level of the organic acid is a median or a mean level of the organic acid of the levels of the organic acid in a cohort of subjects (e.g., a cohort of healthy subjects, a cohort of subjects having moderate CFS, or a cohort of first samples obtained from subjects. In some embodiments, the CFS is associated with an autoimmune disease or an infection.

In some embodiments, the treatment of CFS includes any treatment for reducing any symptom associated with CFS. In some embodiments, the treatment comprises an immunomodulatory compound. In some embodiments, the treatment comprises an immunomodulatory drug (IMiD). IMiDs comprise a group of compounds that can be useful to treat several types of human diseases, including certain cancers. As used herein and unless otherwise indicated, the term “immunomodulatory compound” can encompass certain small organic molecules that inhibit LPS induced monocyte TNF-α, IL-1β, IL-12, IL-6, MIP-1α, MCP-1, GM-CSF, G-CSF, and COX-2 production. These compounds can be prepared synthetically or can be obtained commercially. In other embodiments, the treatment of CFS provided herein comprises a CRBN modulator. In some embodiments, a CRBN modulator is an agent that can modulate at least one of CRBN's biological activities directly or indirectly. In some embodiments, a CRBN modulator is an agent that can physically bind to CRBN. In other embodiments, a CRBN modulator does not directly bind to CRBN, but can otherwise exert an effect via a CRBN mediated pathway. In some embodiments, the treatment of CFS provided herein comprises an agent that depletes B cells. In some embodiments, the agent that depletes B cells is an antibody that specifically binds an antigen of B cells. In some embodiments, the antigen of B cells is CD20, CD19, CD22, CD38, or B-cell activating factor (BAFF). In some embodiments, the agent that depletes B cells is an anti-CD20 antibody. In some embodiments, the anti-CD20 antibody is rituximab, ocrelizumab, or ofatumumab. the agent that depletes B cells is an anti-CD19 antibody. In some embodiments, the anti-CD19 antibody is inebilizumab. In some embodiments, the agent that depletes B cells is an anti-BAFF antibody. In some embodiments, the anti-BAFF antibody is belimumab.

In some embodiments, the treatment compound is formulated in a pharmaceutical composition which comprises a treatment compound provided herein and a pharmaceutically acceptable excipient. In some embodiments, the CFS is associated with an autoimmune disease or an infection.

In some embodiments, when multiple organic acids are used, the second level of each organic acid is compared with a first level of that organic acid, and the method comprises identifying the subject as having CFS if the second level of each organic acid is lower than the first level of that organic acid. In some embodiments, when two or more organic acids are used, a composite score is calculated based on the multiple organic acids and compared with a reference composite score. In some embodiments, the method comprises determining the first and second levels of two or more urine organic acids and obtaining a first composite score based on the first levels of the urine organic acids and a second composite score based on the second levels of the urine organic acids, and comparing the first composite score with the second composite score. In some embodiments, a composite score is calculated using the Median Z-Score method. In other embodiments, a composite score is calculated using the Single-Sample Gene Set Enrichment (ssGSEA) method.

In some embodiments, provided herein is a kit comprising an agent for determining the level of at least one urine organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid. In some embodiments, the organic acid is hippuric acid. In some embodiments, the organic acid is 3-hyroxypropionic acid. In some embodiments, the organic acid is alpha-ketoisocaproic acid. In some embodiments, the organic acid is alpha-keto-beta-methylvaleric acid. In some embodiments, the organic acid is alpha-hydroxybutyric acid. In some embodiments, the organic acid is glycolic acid. In some embodiments, the organic acid is pyruvic acid. In some embodiments, the organic acid is glycolic acid. In some embodiments, the organic acid is citramalic acid. In some embodiments, the organic acid is lactic acid. In some embodiments, the organic acid is alpha-ketoadipic acid. In some embodiments, the organic acid is citric acid. In some embodiments, the organic acid is malic acid. In some embodiments, the organic acid is kynurenic acid. In some embodiments, the organic acid is xanthurenic acid. In some embodiments, the organic acid is isovalerylglycine. In some embodiments, the organic acid is 3-hydroxyisovaleric acid. In some embodiments, the organic acid is isocitric acid. In some embodiments, the organic acid is cis-aconitic acid. In some embodiments, the organic acid is pyroglutamic acid. In some embodiments, the organic acid is vanilmandelic acid. In some embodiments, the organic acid is methylmalonic acid. In some embodiments, the organic acid is glyceric acid. In some embodiments, the kit comprises a tool for obtaining the sample. In some embodiments, the kit comprises an instruction on interpreting the determined level(s). In some embodiments, the CFS is associated with an autoimmune disease or an infection.

### 5.2.2 Biomarkers for Long COVID

Long COVID is also known as Post-COVID Conditions, long-haul COVID, post-acute COVID-19, long-term effects of COVID, and chronic COVID. Long COVID is a wide range of new, returning, or ongoing health problems that people experience after being infected with the virus that causes COVID-19 (see e.g., Thaweethai et al., JAMA (published online May 25, 2023) “Development of a Definition of Postacute Sequelae of SARS-CoV-2 Infection”). Most people with COVID-19 get better within a few days to a few weeks after infection, so at least 3 months after infection is the start of when Long COVID could first be identified. Symptoms of long COVID can include tiredness or fatigue that interferes with daily life, shortness of breath, cough, chest pain, heart palpitations, difficulty thinking or concentrating (sometimes referred to as “brain fog”), headache, sleep problems, dizziness when standing up (lightheadedness), pins-and-needles feelings, change in smell or taste, depression or anxiety, diarrhea, stomach pain, joint or muscle pain, rash, and changes in menstrual cycles. In certain embodiments, the long COVID patients have at least one long COVID symptom at least 3 months, at least 6 months, at least 9 months, at least 12 months, at least 18 months, or at least 2 years after the initial infection with the virus that causes COVID-19. In certain embodiments, the long COVID patients have at least one long COVID symptom at least 6 months after the infection with the virus that causes COVID-19. In some embodiments, the long COVID symptom is selected from fatigue, post-exertional malaise, cognitive dysfunction, sensorimotor symptoms, headache, memory issues, insomnia, muscle aches, heart palpitations, shortness of breath, dizziness and balance issues, speech and language issues, joint pain, and tightness of chest.

In some embodiments, provided herein is a method of identifying a subject having long COVID or verifying long COVID in a subject, the method comprising: (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and (b) identifying or verifying the subject as having long COVID if the expression level of the biomarker is higher than a reference expression level of the biomarker.

In some embodiments, provided herein is a method of identifying a subject who is likely or not likely to be responsive to a treatment of long COVID or predicting the responsiveness of a subject to a treatment of long COVID, the method comprising: (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and (b) identifying or predicting the subject as being likely to be responsive to the treatment if the expression level of the biomarker is higher than a reference expression level of the biomarker. In certain embodiments, the method further comprises administering the treatment to the subject identified or predicted to be likely to be responsive to the treatment.

In some embodiments, provided herein is a method of selectively treating a subject having or suspected of having long COVID with a treatment, the method comprising: (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; (b) identifying or predicting the subject as being likely to be responsive to a treatment of long COVID if the expression level of the biomarker is higher than a reference expression level of the biomarker; and (c) administering the treatment to the subject identified or predicted to be likely to be responsive to the treatment.

In some embodiments, the biomarker is HSPA8. In some embodiments, the biomarker is IKZF3. In some embodiments, the biomarker is ABCE1. In some embodiments, the biomarker is BACH2. In some embodiments, the biomarker is CD3D. In some embodiments, the biomarker is IKZF2.

In some embodiments, the reference expression level of the biomarker is a predetermined expression level of the biomarker. In some embodiments, the reference expression level of the biomarker is a predetermined expression level of the biomarker obtained from a public database. In some embodiments, the reference expression level of the biomarker is an expression level of the biomarker in a healthy subject or a subject who does not have long COVID. In some embodiments, the reference expression level of the biomarker is an expression level of the biomarker in a subject having acute COVID (e.g., severe acute COVID). In some embodiments, the reference expression level of the biomarker is an expression level of the biomarker determined based on a cohort of subjects (e.g., a cohort of healthy subjects, a cohort of subjects not having long COVID, or a cohort of subjects having acute COVID (e.g., severe acute COVID). In some embodiments, the reference expression level of the biomarker is a median or a mean expression level of the biomarker of the expression levels of the biomarker in a cohort of subjects (e.g., a cohort of healthy subjects, a cohort of subjects not having long COVID, a cohort of subjects having acute COVID (e.g., severe acute COVID).

In some embodiments, the biomarker is HSPA8 or IKZF3 and the reference expression level of the biomarker is the expression level of the biomarker in a healthy subject or a subject does not have long COVID, or a cohort of healthy subjects or subjects not having long COVID. In some embodiments, the biomarker is selected from the group consisting of HSPA8, IKZF3, ABCE1, IKZF2, BACH2 and CD3D, and the reference expression level of the biomarker is the expression level of the biomarker in a subject having acute COVID (e.g., severe acute COVID) or a cohort of subjects having acute COVID (e.g., severe acute COVID).

As used herein, a subject having acute COVID refers to a subject was confirmed SARS-CoV-2 infection and is going through the acute phase of the disease, which begins with the appearance of the first symptom associated with SARS-CoV-2 infection and ending 15 days later. A subject having severe acute COVID refers to a subject having acute COVID and with respiratory compromise as defined by requirement of oxygen supplementation but not requiring mechanical ventilation. In certain embodiments, the subject having severe acute COVID were hospitalized (or in the ED awaiting hospitalization).

In some embodiments, the method comprises identifying the subject as having long COVID if the expression level of the biomarker is at least about 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% higher than a reference expression level of the biomarker. The expression level of a biomarker can be determined using any known method in the art, and exemplary methods are described in more detail in Section 5.3 below. In some embodiments, a level is determined to be higher than a reference level if the level is higher (e.g., statistically significantly higher) than the reference level as observed according to a measurement assay.

In certain embodiments, the method comprises using two, three, four, five, or all six biomarkers selected from the group consisting of IKZF2, IKZF3, ABCE1, BACH2, CD3D and HSPA8 to identify or verify a subject having long COVID, or identify or predict a subject as being likely to be responsive to a long COVID treatment. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined.

In some embodiments, when multiple biomarkers are used, the expression level of each biomarker is compared with a reference expression level of that biomarker, and the method comprises identifying or verifying a subject having long COVID, or identifying or predicting a subject as being likely to be responsive to a long COVID treatment if the expression level of each biomarker is higher than the reference expression level of that biomarker. In other embodiments, when multiple biomarkers are used, a composite score is calculated based on the multiple biomarkers and compared with a reference composite score. In some embodiments, the method comprises identifying or verifying a subject having long COVID, or identifying or predicting a subject as being likely to be responsive to a long COVID treatment if the composite score is higher than the reference composite score. In some embodiments, a composite score is calculated using the Median Z-Score method. Briefly, Median Z-Scores are derived by first calculating the mean of each gene from all samples within a gene expression matrix. The mean is then subtracted from each corresponding gene for all samples and then scaling is performed by dividing the values by their standard deviations. The median scaled value from multiple genes of interest comprises the composite score. Another exemplary method for calculating a composite score is the Single-Sample Gene Set Enrichment (ssGSEA) method. Single-sample gene scores represent the degree to which the genes in a particular gene set are coordinately up- or down-regulated within a sample. The score is calculated by adjusting a running-sum statistic based on a decreasing walk through a ranked expression list. The enrichment score is the maximum deviation from zero encountered in the walk; it corresponds to a weighted Kolmogorov-Smirnov-like statistic (see, e.g., Subramanian et al., PNAS, 102 (43): 15545-15550 (2005); and Barbie et al., Nature, 462 (7269): 108-112).

In some embodiments, provided herein is a method of determining or monitoring effectiveness of a treatment in a subject having long COVID, the method comprising: (a) determining a first expression level of a biomarker in a first sample obtained from the subject before administering the treatment to the subject, wherein the biomarker is a cereblon (CRBN)-associated protein (CAP) selected from the group consisting of HSPA8, IKZF3, ABCE1, IKZF2, BACH2 and CD3D; (b) administering the treatment to the subject; (c) determining a second expression level of the biomarker in a second sample obtained from the subject after administering the treatment to the subject; and (d) determining the effectiveness of the treatment based on the comparison of the first expression level with the second expression level. In certain embodiments, the method comprises determining that the treatment is effective if the second expression level is lower than the first expression level. In certain embodiments, the method comprises determining that the treatment is not effective if the second expression level is not lower than the first expression level. In certain embodiments, the method further comprises determining or adjusting (e.g., increasing) the dose of the treatment or administering a different long COVID treatment to the subject if the second expression level is not lower than the first expression level.

In some embodiments, provided herein is a method of screening a treatment for effectiveness in treating long COVID, the method comprising: (a) determining a first expression level of a biomarker in a sample before administering the compound to the sample, wherein the biomarker is a cereblon (CRBN)-associated protein (CAP) selected from the group consisting of HSPA8, IKZF3, ABCE1, IKZF2, BACH2 and CD3D; (b) administering the treatment to the sample; (c) determining a second expression level of the biomarker in the sample after administering the treatment to the sample; (d) comparing the first expression level with the second expression level; and (e) selecting the treatment if the second expression level is lower than the first expression level.

In some embodiments, the method comprises determining that the treatment is effective if the second expression level is at least 10%, at least 20%, at least 30%, at least 40%, at least 50%, at least 60%, at least 70%, at least 80%, or at least 90% lower than the first expression level. Based on the comparison of the first expression level and the second expression level, a different treatment or a different dosing regimen may be administered to the subject in the subsequent treatment cycle(s).

In some embodiments, the biomarker is IKZF2. In some embodiments, the biomarker is IKZF3. In some embodiments, the biomarker is ABCE1. In some embodiments, the biomarker is BACH2. In some embodiments, the biomarker is CD3D. In some embodiments, the biomarker is HSPA8.

The expression level of a biomarker can be determined using any known method in the art, and exemplary methods are described in more detail in Section 5.3 below. In some embodiments, a level is determined to be higher than a second level if the level is higher (e.g., statistically significantly higher) than the second level as observed according to a measurement assay. In some embodiments, a level is determined to be lower than a second level if the level is lower (e.g., statistically significantly higher) than the second level as observed according to a measurement assay.

In certain embodiments, the method comprises using two, three, four, five, or all six biomarkers selected from the group consisting of IKZF2, IKZF3, ABCE1, BACH2, CD3D and HSPA8. In some embodiments, the expression levels of IKZF2 and IKZF3 are determined. In some embodiments, the expression levels of IKZF2 and ABCE1 are determined. In some embodiments, the expression levels of IKZF2 and BACH2 are determined. In some embodiments, the expression levels of IKZF2 and CD3D are determined. In some embodiments, the expression levels of IKZF2 and HSPA8 are determined. In some embodiments, the expression levels of IKZF3 and ABCE1 are determined. In some embodiments, the expression levels of IKZF3 and BACH2 are determined. In some embodiments, the expression levels of IKZF3 and CD3D are determined. In some embodiments, the expression levels of IKZF3 and HSPA8 are determined. In some embodiments, the expression levels of ABCE1 and BACH2 are determined. In some embodiments, the expression levels of ABCE1 and CD3D are determined. In some embodiments, the expression levels of ABCE1 and HSPA8 are determined. In some embodiments, the expression levels of BACH2 and CD3D are determined. In some embodiments, the expression levels of BACH2 and HSPA8 are determined. In some embodiments, the expression levels of CD3D and HSPA8 are determined. In some embodiments, the expression levels of IKZF2, IKZF3 and ABCE1 are determined. In some embodiments, the expression levels of IKZF2, IKZF3 and BACH2 are determined. In some embodiments, the expression levels of IKZF2, IKZF3 and CD3D are determined. In some embodiments, the expression levels of IKZF2, IKZF3 and HSPA8 are determined. In some embodiments, the expression levels of IKZF2, ABCE1 and BACH2 are determined. In some embodiments, the expression levels of IKZF2, ABCE1 and CD3D are determined. In some embodiments, the expression levels of IKZF2, ABCE1 and HSPA8 are determined. In some embodiments, the expression levels of IKZF2, BACH2 and CD3D are determined. In some embodiments, the expression levels of IKZF2, BACH2 and HSPA8 are determined. In some embodiments, the expression levels of IKZF2, CD3D and HSPA8 are determined. In some embodiments, the expression levels of IKZF3, ABCE1 and BACH2 are determined. In some embodiments, the expression levels of IKZF3, ABCE1 and CD3D are determined. In some embodiments, the expression levels of IKZF3, ABCE1 and HSPA8 are determined. In some embodiments, the expression levels of IKZF3, BACH2 and CD3D are determined. In some embodiments, the expression levels of IKZF3, BACH2 and HSPA8 are determined. In some embodiments, the expression levels of IKZF3, CD3D and HSPA8 are determined. In some embodiments, the expression levels of ABCE1, BACH2 and CD3D are determined. In some embodiments, the expression levels of ABCE1, BACH2 and HSPA8 are determined. In some embodiments, the expression levels of ABCE1, CD3D and HSPA8 are determined. In some embodiments, the expression levels of BACH2, CD3D and HSPA8 are determined. In some embodiments, the expression levels of ABCE1, BACH2, CD3D and HSPA8 are determined. In some embodiments, the expression levels of IKZF3, BACH2, CD3D and HSPA8 are determined. In some embodiments, the expression levels of IKZF3, ABCE1, CD3D and HSPA8 are determined. In some embodiments, the expression levels of IKZF3, ABCE1, BACH2 and HSPA8 are determined. In some embodiments, the expression levels of IKZF3, ABCE1, BACH2 and CD3D are determined. In some embodiments, the expression levels of IKZF2, BACH2, CD3D and HSPA8 are determined. In some embodiments, the expression levels of IKZF2, ABCE1, CD3D and HSPA8 are determined. In some embodiments, the expression levels of IKZF2, ABCE1, BACH2 and HSPA8 are determined. In some embodiments, the expression levels of IKZF2, ABCE1, BACH2 and CD3D are determined. In some embodiments, the expression levels of IKZF2, IKZF3, CD3D and HSPA8 are determined. In some embodiments, the expression levels of IKZF2, IKZF3, BACH2 and HSPA8 are determined. In some embodiments, the expression levels of IKZF2, IKZF3, BACH2 and CD3D are determined. In some embodiments, the expression levels of IKZF2, IKZF3, ABCE1 and HSPA8 are determined. In some embodiments, the expression levels of IKZF2, IKZF3, ABCE1 and CD3D are determined. In some embodiments, the expression levels of IKZF2, IKZF3, ABCE1 and BACH2 are determined. In some embodiments, the expression levels of IKZF3, ABCE1, BACH2, CD3D and HSPA8 are determined. In some embodiments, the expression levels of IKZF2, ABCE1, BACH2, CD3D and HSPA8 are determined. In some embodiments, the expression levels of IKZF2, IKZF3, BACH2, CD3D and HSPA8 are determined. In other embodiments, the expression levels of IKZF2, IKZF3, ABCE1, CD3D and HSPA8 are determined. In other embodiments, the expression levels of IKZF2, IKZF3, ABCE1, BACH2 and HSPA8 are determined. In other embodiments, the expression levels of IKZF2, IKZF3, ABCE1, BACH2 and CD3D are determined. In some embodiments, the expression levels of IKZF2, IKZF3, ABCE1, BACH2, CD3D and HSPA8 are determined.

In some embodiments, when multiple biomarkers are used, the second expression level of each biomarker is compared with a first expression level of that biomarker, and the method comprises determining that the treatment is effective if the second expression level of each biomarker is lower than the first expression level of that biomarker. In other embodiments, when multiple biomarkers are used, a composite score is calculated based on the multiple biomarkers and compared with a reference composite score. In some embodiments, a composite score is calculated using the Median Z-Score method. In other embodiments, a composite score is calculated using the Single-Sample Gene Set Enrichment (ssGSEA) method.

In certain embodiments, the treatment for long COVID disclosed herein comprises an immunomodulatory drug (IMiD). IMiDs comprise a group of compounds that can be useful to treat several types of human diseases, including certain cancers. As used herein and unless otherwise indicated, the term “immunomodulatory compound” can encompass certain small organic molecules that inhibit LPS induced monocyte TNF-α, IL-1β, IL-12, IL-6, MIP-1α, MCP-1, GM-CSF, G-CSF, and COX-2 production. These compounds can be prepared synthetically or can be obtained commercially. The inflammatory cytokine TNF-α, which is produced by macrophages and monocytes during acute inflammation, causes a diverse range of signaling events within cells. Without being limited by a particular theory, one of the biological effects exerted by the immunomodulatory compounds disclosed herein is the reduction of myeloid cell TNF-α production. Immunomodulatory compounds disclosed herein may enhance the degradation of TNF-α mRNA. Further, without being limited by theory, immunomodulatory compounds disclosed herein may also be potent co-stimulators of T cells and increase cell proliferation dramatically in a dose dependent manner. Immunomodulatory compounds disclosed herein may also have a greater co-stimulatory effect on the CD8+ T cell subset than on the CD4+ T cell subset. In addition, the compounds may have anti-inflammatory properties against myeloid cell responses, yet efficiently co-stimulate T cells to produce greater amounts of IL-2, IFN-γ, and to enhance T cell proliferation and CD8+ T cell cytotoxic activity. Further, without being limited by a particular theory, immunomodulatory compounds disclosed herein may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-7, and to enhance NK and NKT cell cytotoxic activity. Various immunomodulatory compounds disclosed herein contain one or more chiral centers, and can exist as racemic mixtures of enantiomers or mixtures of diastereomers. Thus, also provided herein is the use of stereomerically pure forms of such compounds, as well as the use of mixtures of those forms. For example, mixtures comprising equal or unequal amounts of the enantiomers of a particular immunomodulatory compounds may be used. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

In certain embodiments, the treatment for long COVID disclosed herein comprises a CRBN modulator. In some embodiments, a CRBN modulator is an agent that can modulate at least one of CRBN's biological activities directly or indirectly. In some embodiments, a CRBN modulator is an agent that can physically bind to CRBN. In other embodiments, a CRBN modulator does not directly bind to CRBN, but can otherwise exert an effect via a CRBN mediated pathway. Cereblon (CRBN), a component of the DDB1-CUL4a-Roc1 ubiquitin ligase complex, has been identified as a target of certain immunomodulatory compounds, e.g., thalidomide, lenalidomide, and pomalidomide, and iberdomide (Lopez-Girona et al., Leukemia volume 26, pages 2326-2335 (2012); Bjorklund et al., Leukemia. 2020; 34(4): 1197-1201). It is believed that the interactions of CRBN with certain immunomodulatory compounds mediate their anti-proliferative effects in multiple myeloma (MM) cells (Lopez-Girona et al., Leukemia 2012; Zhu et al., Blood 2011, 118, Abstract 127). CRBN is encoded by a 25 kb gene on chromosome 6, consisting of 11 exons and 10 introns. Thus, alternative splicing process can potentially generate multiple functional proteins as well as variants of a protein from a single gene having different structural organization and functional activity. Truncated proteins that have lost interaction domains or critical functional amino acid residues may create non-functional or aberrant CRBN proteins that may interfere with the functions of the full-length CRBN protein and reduce or alter the therapeutic activity of a treatment compound that exerts its activity via its interactions with the full-length CRBN protein. At least two isoforms of the protein cereblon (CRBN) exist, which are 442 and 441 amino acids long, respectively, and CRBN is conserved from plant to human. In humans, the CRBN gene has been identified as a candidate gene of an autosomal recessive nonsyndromic mental retardation (ARNSMR). See Higgins, J. J. et al., Neurology, 2004, 63:1927-1931. CRBN was initially characterized as an RGS-containing novel protein that interacted with a calcium-activated potassium channel protein (SLO1) in the rat brain, and was later shown to interact with a voltage-gated chloride channel (CIC-2) in the retina with AMPK1 and DDB1. See Jo, S. et al., J. Neurochem, 2005, 94:1212-1224; Hohberger B. et al., FEBS Lett, 2009, 583:633-637; Angers S. et al., Nature, 2006, 443:590-593. DDB1 was originally identified as a nucleotide excision repair protein that associates with damaged DNA binding protein 2 (DDB2). Its defective activity causes the repair defect in the patients with xeroderma pigmentosum complementation group E (XPE). DDB1 also appears to function as a component of numerous distinct DCX (DDB1-CUL4-X-box) E3 ubiquitin-protein ligase complexes which mediate the ubiquitination and subsequent proteasomal degradation of target proteins. CRBN has also been identified as a target for the development of therapeutic agents for diseases of the cerebral cortex. See WO 2010/137547 A1. In some embodiments, binding to CRBN or one or more substrates of CRBN is required for the beneficial effects of certain treatment compounds provided herein. In some embodiments, the treatment compound provided herein can induce CRBN to undergo conformational changes. In some embodiments, the use of a treatment compound provided herein leads to a distinct conformational change or other alteration in the properties of the CRBN surface, and a resulting distinct phenotypic response. In certain embodiments, a CRBN modulator is an immunomodulatory compound. In other embodiments, a CRBN is not an immunomodulatory compound.

In some embodiments, the treatment for long COVID disclosed herein comprises an agent that depletes B cells. In some embodiments, the agent that depletes B cells is an antibody that specifically binds an antigen of B cells. In some embodiments, the antigen of B cells is CD20, CD19, CD22, CD38, or B-cell activating factor (BAFF). In some embodiments, the agent that depletes B cells is an anti-CD20 antibody. In some embodiments, the anti-CD20 antibody is rituximab, ocrelizumab, or ofatumumab. the agent that depletes B cells is an anti-CD19 antibody. In some embodiments, the anti-CD19 antibody is inebilizumab. In some embodiments, the agent that depletes B cells is an anti-BAFF antibody. In some embodiments, the anti-BAFF antibody is belimumab.

In some embodiments, the treatment compound provided herein is formulated in a pharmaceutical composition which comprises a treatment compound provided herein and a pharmaceutically acceptable excipient. Pharmaceutical compositions comprising treatment compounds provided herein are prepared for storage by mixing the compound provided herein with optional physiologically acceptable excipients (see, e.g., Remington, Remington's Pharmaceutical Sciences (18th ed. 1980)) in the form of aqueous solutions or lyophilized or other dried forms. The treatment compound of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45). The treatment compound provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra. Various compositions and delivery systems are known and can be used with a compound as described herein. In some embodiments, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising the treatment compound described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60). Various delivery systems are known and can be used to administer a treatment compound provided herein.

### 5.3. Methods of Detecting and Quantifying Biomarkers

Any methods and technologies known in the art for detecting or quantifying a biomarker may be used in the present methods. In certain embodiments, the expression level of a biomarker is determined by measuring the nucleic acid level of the biomarker. In certain embodiments, the expression level of a biomarker provided herein is determined by measuring the mRNA level of the biomarker. In other embodiments, the expression level of a biomarker provided herein is determined by measuring cDNA level of the biomarker. In other embodiments, the expression level of a biomarker provided herein is determined by measuring the protein level of the biomarker.

Several methods of detecting or quantitating mRNA levels are known in the art. Exemplary methods include, but are not limited to, northern blots, RNA sequencing, microarray, ribonuclease protection assays, PCR-based methods, and the like. The mRNA sequence of a biomarker can be used to prepare a probe that is at least partially complementary to the mRNA sequence. The probe can then be used to detect the mRNA in a sample, using any suitable assay, such as PCR-based methods, northern blotting, a dipstick assay, and the like.

In other embodiments, a nucleic acid assay for detecting or quantitating a biomarker in a biological sample can be prepared. The assay can include a solid support and at least one nucleic acid contacting the support, where the nucleic acid corresponds to at least a portion of an mRNA of a biomarker. The assay can also have a means for detecting the expression (e.g., altered expression) of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample. Exemplary methods also include Next Generation Sequencing (NGS).

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs.

In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3^{rd} ed. 1995); Sambrook et al., Molecular Cloning: A Laboratory Manual (Cold Spring Harbor, N.Y., 3^{rd} ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5-carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258), phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

The nucleic acids may be present in specific, addressable locations on a solid support, each corresponding to at least a portion of mRNA sequences that are differentially expressed upon treatment of a compound in a cell or a patient.

For example, in certain embodiments, a method of detecting and quantifying the RNA (e.g., mRNA) level of a biomarker from a biological sample comprises: (a) obtaining RNA from the sample; (b) contacting the RNA with a primer that specifically binds to a sequence in the RNA to generate a first DNA molecule having a sequence complementary to said RNA; (c) amplifying the DNA corresponding to a segment of a gene encoding the biomarker; and (d) determining the RNA level of the biomarker based on the amount of the amplified DNA.

A typical mRNA assay method can contain the steps of 1) obtaining surface-bound subject probes; 2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g., under conditions sufficient to provide for specific binding of target mRNAs in the sample to the probes) are described in Kallioniemi et al., Science 1992, 258:818-821 and International Patent Application Publication No. WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al., Meth. Enzymol. 1981, 21:470-480; Angerer et al., Genetic Engineering: Principles and Methods, Vol 7, pgs 43-65 (Plenum Press, New York, Setlow and Hollaender, eds. 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to detect or qualify the expression of a biomarker. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using 7500 Real-Time PCR System Sequence Detection software vs. using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

Several protein detection and quantization methods can be used to measure the level of a biomarker. Any suitable protein quantization method can be used. In some embodiments, antibody-based methods are used. In some embodiments, the protein level of a biomarker is measured using an immunoassay. In some embodiments, the immunoassay comprises Western blots, enzyme-linked immunosorbent assay (ELISA), flow cytometry, immunoprecipitation, immunohistochemistry, immunofluorescence, radioimmunoassay (RIA), dot blotting, and flow cytometry. In some embodiments, the ELISA is direct ELISA (enzyme-linked immunosorbent assay), indirect ELISA, sandwich ELISA, competitive ELISA, multiplex ELISA, ELISPOT technologies, and other similar techniques known in the art.

In some embodiments, the protein level of a biomarker is determined by using mass spectrometry (MS). In some embodiments, MS comprises liquid chromatography-tandem mass spectrometry (LC MS/MS), liquid chromatography-mass spectrometry (LC-MS), multiple reaction monitoring (MRM), selected reaction monitoring (SRM), affinity-capture MS (AC-MS), matrix-assisted laser desorption/ionization time-of-flight (MALDI-TOF) MS, MALDI-TOF post-source-decay (PSD), MALDI-TOF/TOF, surface-enhanced laser desorption/ionization time-of-flight mass spectrometry (SELDI-TOF) MS, electrospray ionization mass spectrometry (ESI-MS), ESI-MS/MS, ESI-MS/(MS)n (n is an integer greater than zero), ESI 3D or linear (2D) ion trap MS, ESI triple quadrupole MS, ESI quadrupole orthogonal TOF (Q-TOF), ESI Fourier transform MS systems, desorption/ionization on silicon (DIOS), secondary ion mass spectrometry (SIMS), atmospheric pressure chemical ionization mass spectrometry (APCI-MS), APCI-MS/MS, APCI-(MS)n, ion mobility spectrometry (IMS), inductively coupled plasma mass spectrometry (ICP-MS)atmospheric pressure photoionization mass spectrometry (APPI-MS), APPI-MS/MS, and APPI-(MS)n.

An exemplary antibody based assay for determining a protein level of a biomarker comprises contacting proteins within the sample with a first antibody that immunospecifically binds to the biomarker protein. In some embodiments, the methods provided herein further comprise (i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of the second antibody bound to the biomarker protein; and (iii) determining the amount of the biomarker protein based on the amount of detectable label in the second antibody. In other embodiments, the methods provided herein further comprise (i) contacting the biomarker protein bound to the first antibody with a second antibody with a detectable label, wherein the second antibody immunospecifically binds to the first antibody; (ii) detecting the presence of the second antibody bound to the first antibody; and (iii) determining the amount of the biomarker protein based on the amount of detectable label in the second antibody.

In certain embodiments of the various methods provided herein, the two or more of the steps are performed sequentially. In other embodiments of the methods provided herein, two or more of steps are performed in parallel (e.g., at the same time).

### 5.4. Subjects, Samples, and Types of Cells

In certain embodiments, the various methods provided herein use samples (e.g., biological samples) from subjects or individuals (e.g., patients). The subject can be a patient, such as, e.g., a patient with a CFS. The subject can be a mammal, for example, a human. The subject can be male or female, and can be an adult, a child, or an infant. Samples can be analyzed at a time during an active phase of CFS, or when the CFS is inactive. In certain embodiments, more than one sample from a subject is obtained.

In some embodiments, the subject (e.g., patient) has one or more symptoms of CFS. In some embodiments, the subject is a patient with fatigue, e.g., a bedridden patient with fatigue. In some embodiments, the fatigue was triggered by an infection, e.g., a viral or bacterial infection. In some embodiments, the patient has or has had EBV, CMV, HHV-6, HHV-7. In some embodiments, the patient is positive for EBV, CMV, HHV-6A, HHV-6B, or HHV-7 (e.g., based on detection of viral DNA, e.g., in a blood sample, e.g., in a plasma sample). In some embodiments, the patient has been infected with a coronavirus (e.g., SARS-CoV-2). In some embodiments, the patient has had symptoms of COVID-19.

In some embodiments, the patient has been diagnosed with an autoimmune disease. In some embodiments, the patient has or has had an autoimmune disease. In some embodiments, the autoimmune disease is Hashimoto's thyroiditis, fibromyalgia, inflammatory bowel disease (IBD) (e.g., Crohn's disease or ulcerative colitis), postural orthostatic tachycardia syndrome (POTS), Grave's Disease, psoriasis, rheumatoid arthritis or Sjogren's syndrome. In some embodiments, the patient is positive for, or has tested positive for, autoantibodies indicative of an autoimmune disease.

In certain embodiments, the sample used in the methods provided herein comprises body fluids from a subject. Non-limiting examples of body fluids include blood (e.g., whole blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids (including cerebrospinal fluid surrounding the brain and the spinal cord), synovial fluid, intracellular fluid (the fluid inside cells), and vitreous humour (the fluid in the eyeball). In some embodiments, the sample is a blood sample. The blood sample can be obtained using conventional techniques as described in, e.g., Innis et al, eds., PCR Protocols (Academic Press, 1990). White blood cells can be separated from blood samples using conventional techniques or commercially available kits, e.g., RosetteSep kit (Stein Cell Technologies, Vancouver, Canada). Sub-populations of white blood cells, e.g., mononuclear cells, B cells, T cells, monocytes, granulocytes, or lymphocytes, can be further isolated using conventional techniques, e.g., magnetically activated cell sorting (MACS) (Miltenyi Biotec, Auburn, California) or fluorescently activated cell sorting (FACS) (Becton Dickinson, San Jose, California).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3.0 mL. In another embodiment, the blood sample is about 0.3 mL, about 0.4 mL, about 0.5 mL, about 0.6 mL, about 0.7 mL, about 0.8 mL, about 0.9 mL, about 1.0 mL, about 1.5 mL, about 2.0 mL, about 2.5 mL, about 3.0 mL, about 3.5 mL, about 4.0 mL, about 4.5 mL, about 5.0 mL, about 6.0 mL, about 7.0 mL, about 8.0 mL, about 9.0 mL, or about 10.0 mL.

In some embodiments, the sample used in the present methods comprises a biopsy. The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the subject receiving a treatment for the disease or disorder. In another embodiment, the sample is obtained from the subject during the subject receiving a treatment for the disease or disorder. In another embodiment, the sample is obtained from the subject after the subject receiving a treatment for the disease or disorder. In various embodiments, the treatment comprises administering a compound to the subject.

In certain embodiments, the sample used in the methods provided herein comprises a plurality of cells. In certain embodiments, the number of cells used in the methods provided herein can range from a single cell to about 10⁹ cells. In some embodiments, the number of cells used in the methods provided herein is about 1×10⁴ cells, about 5×10⁴ cells, about 1×10⁵ cells, about 5×10⁵ cells, about 1×10⁶ cells, about 5×10⁶ cells, about 1×10⁷ cells, about 5×10⁷ cells, about 1×10⁸ cells, about 5×10⁸ cells, or about 1×10⁹ cells.

The number and type of cells collected from a subject can be monitored, for example, by measuring changes in cell surface markers using standard cell detection techniques such as flow cytometry, cell sorting, immunocytochemistry (e.g., staining with tissue specific or cell-marker specific antibodies), fluorescence activated cell sorting (FACS), magnetic activated cell sorting (MACS), by examining the morphology of cells using light or confocal microscopy, and/or by measuring changes in gene expression using techniques well known in the art, such as PCR and gene expression profiling. These techniques can be used, too, to identify cells that are positive for one or more particular markers.

In certain embodiments, subsets of cells are used in the methods provided herein. Methods of sorting and isolating specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc. Fluorescence activated cell sorting (FACS) is a well-known method for separating particles, including cells, based on the fluorescent properties of the particles (Kamarch, Methods Enzymol. 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one embodiment, RNA (e.g., mRNA) or protein is purified, and the presence or absence of a biomarker is measured by gene or protein expression analysis. In certain embodiments, the presence or absence of a biomarker is measured by quantitative real-time PCR (qRT-PCR), microarray, flow cytometry, or immunofluorescence. In other embodiments, the presence or absence of a biomarker is measured by ELISA or other similar methods known in the art. Other exemplary methods are described in Section 5.3 above.

### 5.5. Kits

In another aspect, provided herein is a kit for performing a method provided herein. In some embodiments, provided herein is a kit for identifying a subject having CFS or verifying CFS in a subject, comprising an agent for obtaining an expression level of a biomarker in a sample from the subject, wherein the biomarker is selected from the group consisting of IKZF2, IKZF3, ABCE1, BACH2, CD3D and HSPA8. In some embodiments, the kit further comprises an instruction for identifying or verifying the subject as having CFS if the expression level of the biomarker is higher than a reference expression level of the biomarker. In some embodiments, the kit (for example, in the instruction) provides the reference expression level of the biomarker. In some embodiments, the kit further comprises a tool for obtaining a sample from a subject.

In some embodiments, provided herein is a kit for determining severity of CFS in a subject comprising an agent for obtaining an expression level of a biomarker in a sample from the subject, wherein the biomarker is selected from the group consisting of IKZF2, IKZF3, ABCE1, BACH2, CD3D and HSPA8. In some embodiments, the kit further comprises an instruction on how to determine severity of CFS. In some embodiments, the kit (for example, in the instruction) provides the reference expression level of the biomarker. In some embodiments, the kit provides an instruction on comparing the expression level of the biomarker with the reference level of the biomarker and determining the severity of CFS based on the comparison. In some embodiments, the kit further comprises a tool for obtaining a sample from a subject.

In some embodiments, provided herein is a kit of identifying a subject who is likely or not likely to be responsive to a treatment of CFS or predicting the responsiveness of a subject to a treatment of CFS comprising an agent for obtaining an expression level of a biomarker in a sample from the subject, wherein the biomarker is selected from the group consisting of IKZF2, IKZF3, ABCE1, BACH2, CD3D and HSPA8. In some embodiments, the kit further comprises an instruction on how to identify a subject who is likely or not likely to be responsive to a treatment of CFS or predict the responsiveness of a subject to a treatment of CFS. In some embodiments, the kit (for example, in the instruction) provides the reference expression level of the biomarker. In some embodiments, the kit provides an instruction on identifying or predicting the subject as being likely to be responsive to a treatment of CFS if the expression level of the biomarker is higher than a reference expression level of the biomarker. In some embodiments, the kit further comprises a tool for obtaining a sample from a subject. In some embodiments, the kit is for identifying a subject who is likely or not likely to be responsive to an immunomodulatory drug (IMiD) or predicting the responsiveness of a subject to an IMiD. In other embodiments, the kit is for identifying a subject who is likely or not likely to be responsive to a CRBN modulator or a compound capable of binding and/or inducing conformational change to CRBN or predicting the responsiveness of a subject to a CRBN modulator or a compound capable of binding and/or inducing conformational change to CRBN. In some embodiments, the kit is for identifying a subject who is likely or not likely to be responsive to an agent that depletes B cells (e.g., an anti-CD20 antibody, e.g., rituximab) or predicting the responsiveness of a subject to an agent that depletes B cells (e.g., an anti-CD20 antibody, e.g., rituximab).

In some embodiments of the various kits provided herein, the reference expression level of the biomarker provided in the kit is a predetermined expression level of the biomarker from a public database. In some embodiments of the various kits provided herein, the reference expression level of the biomarker provided in the kit is an expression level of the biomarker in a healthy subject or in a subject who does not have CFS. In some embodiments, the reference expression level of the biomarker is an expression level of the biomarker in a subject having moderate CFS. In some embodiments of the various kits provided herein, the reference expression level of the biomarker provided in the kit is an expression level of the biomarker determined based on a cohort of subjects (e.g., a cohort of healthy subjects, a cohort of subjects not having CFS, or a cohort of subjects having moderate CFS). In some embodiments, the reference expression level of the biomarker is a median or a mean expression level of the biomarker of the expression levels of the biomarker in a cohort of subjects (e.g., a cohort of healthy subjects, a cohort of subjects not having CFS, or a cohort of subjects having moderate CFS). In some embodiments, the biomarker is HSPA8 or ABCE1 and the reference expression level of the biomarker is the expression level of the biomarker in a healthy subject or a subject does not have CFS, or a cohort of healthy subjects or subjects not having CFS. In some embodiments, the biomarker is selected from the group consisting of IKZF2, IKZF3, ABCE1, BACH2, CD3D and HSPA8, and the reference expression level of the biomarker is the expression level of the biomarker in a subject having mild CFS or a cohort of subjects having mild CFS.

In other embodiments, provided herein is a kit for determining or monitoring effectiveness of a treatment in a subject having CFS comprising an agent for obtaining a first expression level of a biomarker in a first sample from the subject, wherein the biomarker is selected from the group consisting of IKZF2, IKZF3, ABCE1, BACH2, CD3D and HSPA8; and an agent for obtaining a second expression level of the biomarker in a second sample obtained from the subject after administering a treatment to the subject. In some embodiments, the kit further comprises an instruction on how to determine or monitor effectiveness of a treatment in a subject having CFS. In some embodiments, the kit provides an instruction on determining the effectiveness of the treatment based on the comparison of the first expression level with the second expression level. In some embodiments, the kit provides an instruction on determining that the treatment is effective if the second expression level is lower than the first expression level. In some embodiments, the kit further comprises a tool for obtaining a sample from a subject. In some embodiments, the kit is for determining or monitoring effectiveness of an immunomodulatory drug (IMiD) in a subject having CFS. In some embodiments, the kit is for determining or monitoring effectiveness of a CRBN modulator or a compound capable of binding and/or inducing conformational change to CRBN in a subject having CFS. In some embodiments, the kit is for determining or monitoring effectiveness of an agent that depletes B cells (e.g., an anti-CD20 antibody, e.g., rituximab) in a subject having CFS. In some embodiments, the kit further comprises an instruction on determining or adjusting a dose of the treatment to the subject.

In yet other embodiments, provided herein is a kit for screening a compound for effectiveness in treating CFS comprising an agent for obtaining a first level of a biomarker in a sample, wherein the biomarker is selected from the group consisting of IKZF2, IKZF3, ABCE1, BACH2, CD3D and HSPA8; and an agent for obtaining a second level of the biomarker in the sample after administering the compound to the sample. In some embodiments, the kit further comprises an instruction on how to select a compound for treating CFS. In some embodiments, the kit provides an instruction on selecting the compound if the second level is lower than the first level. In some embodiments, the kit further comprises a tool for obtaining a sample. In some embodiments, the kit is for screening an immunomodulatory drug (IMiD) for effectiveness in treating CFS. In some embodiments, the kit is for screening a CRBN modulator or a compound capable of binding and/or inducing conformational change to CRBN for effectiveness in treating CFS. In some embodiments, the kit is for screening an agent that depletes B cells (e.g., an anti-CD20 antibody, e.g., rituximab) for effectiveness in treating CFS.

In yet other embodiments, provided herein is a kit for identifying a subject having long COVID or verifying long COVID in a subject, comprising an agent for determining an expression level of a biomarker in a sample from the subject, wherein the biomarker is a CAP selected from the group consisting of HSPA8, IKZF3, ABCE1, IKZF2, BACH2 and CD3D. In some embodiments, the kit further comprises an instruction for identifying or verifying the subject as having long COVID if the expression level of the biomarker is higher than a reference expression level of the biomarker. In some embodiments, the kit provides the reference expression level of the biomarker. In some embodiments, the kit further comprises a tool for obtaining a sample from a subject.

In some embodiments, provided herein is a kit for identifying a subject who is likely or not likely to be responsive to a treatment of long COVID or predicting the responsiveness of a subject to a treatment of long COVID, comprising an agent for determining an expression level of a biomarker in a sample from the subject, wherein the biomarker is a CAP selected from the group consisting of HSPA8, IKZF3, ABCE1, IKZF2, BACH2 and CD3D. In some embodiments, the kit further comprises an instruction for identifying or predicting the subject as being likely to be responsive to the treatment if the expression level of the biomarker is higher than a reference expression level of the biomarker. In certain embodiments, the kit further comprises instructions for administering the treatment to the subject identified or predicted to be likely to be responsive to the treatment. In some embodiments, the kit provides the reference expression level of the biomarker. In some embodiments, the kit further comprises a tool for obtaining a sample from a subject.

In some embodiments, provided herein is a kit for selectively treating a subject having or suspected of having long COVID with a treatment, comprising an agent for determining an expression level of a biomarker in a sample from the subject, wherein the biomarker is a CAP selected from the group consisting of HSPA8, IKZF3, ABCE1, IKZF2, BACH2 and CD3D. In some embodiments, the kit further comprises an instruction for identifying or predicting the subject as being likely to be responsive to a treatment of long COVID if the expression level of the biomarker is higher than a reference expression level of the biomarker. In certain embodiments, the kit further comprises instructions for administering the treatment to the subject identified or predicted to be likely to be responsive to the treatment. In some embodiments, the kit provides the reference expression level of the biomarker. In some embodiments, the kit further comprises a tool for obtaining a sample from a subject.

In some embodiments of the various kits provided herein, the reference expression level of the biomarker is a predetermined expression level of the biomarker. In some embodiments, the reference expression level of the biomarker is a predetermined expression level of the biomarker obtained from a public database. In some embodiments, the reference expression level of the biomarker is an expression level of the biomarker in a healthy subject or a subject who does not have long COVID. In some embodiments, the reference expression level of the biomarker is an expression level of the biomarker in a subject having acute COVID (e.g., severe acute COVID). In some embodiments, the reference expression level of the biomarker is an expression level of the biomarker determined based on a cohort of subjects (e.g., a cohort of healthy subjects, a cohort of subjects not having long COVID, or a cohort of subjects having acute COVID (e.g., severe acute COVID). In some embodiments, the reference expression level of the biomarker is a median or a mean expression level of the biomarker of the expression levels of the biomarker in a cohort of subjects (e.g., a cohort of healthy subjects, a cohort of subjects not having long COVID, a cohort of subjects having acute COVID (e.g., severe acute COVID).

In some embodiments of the various kits provided herein, the biomarker is HSPA8 or IKZF3 and the reference expression level of the biomarker is the expression level of the biomarker in a healthy subject or a subject does not have long COVID, or a cohort of healthy subjects or subjects not having long COVID. In some embodiments, the biomarker is selected from the group consisting of HSPA8, IKZF3, ABCE1, IKZF2, BACH2 and CD3D, and the reference expression level of the biomarker is the expression level of the biomarker in a subject having acute COVID (e.g., severe acute COVID) or a cohort of subjects having acute COVID (e.g., severe acute COVID).

In some embodiments of the various kits provided herein, the kit comprises identifying the subject as having long COVID if the expression level of the biomarker is at least about 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1000% higher than a reference expression level of the biomarker. The expression level of a biomarker can be determined using any known method in the art, and exemplary methods are described in more detail in Section 5.3 below. In some embodiments, a level is determined to be higher than a reference level if the level is higher (e.g., statistically significantly higher) than the reference level as observed according to a measurement assay.

In some embodiments of the various kits provided herein, when multiple biomarkers are used, the expression level of each biomarker is compared with a reference expression level of that biomarker, and the kit comprises instructions for identifying or verifying a subject having long COVID, or identifying or predicting a subject as being likely to be responsive to a long COVID treatment if the expression level of each biomarker is higher than the reference expression level of that biomarker. In other embodiments, when multiple biomarkers are used, a composite score is calculated based on the multiple biomarkers and compared with a reference composite score. In some embodiments, the kit comprises instructions for identifying or verifying a subject having long COVID, or identifying or predicting a subject as being likely to be responsive to a long COVID treatment if the composite score is higher than the reference composite score. In some embodiments, a composite score is calculated using the Median Z-Score method. Briefly, Median Z-Scores are derived by first calculating the mean of each gene from all samples within a gene expression matrix. The mean is then subtracted from each corresponding gene for all samples and then scaling is performed by dividing the values by their standard deviations. The median scaled value from multiple genes of interest comprises the composite score. Another exemplary method for calculating a composite score is the Single-Sample Gene Set Enrichment (ssGSEA) method. Single-sample gene scores represent the degree to which the genes in a particular gene set are coordinately up- or down-regulated within a sample. The score is calculated by adjusting a running-sum statistic based on a decreasing walk through a ranked expression list. The enrichment score is the maximum deviation from zero encountered in the walk; it corresponds to a weighted Kolmogorov-Smirnov-like statistic (see, e.g., Subramanian et al., PNAS, 102 (43): 15545-15550 (2005); and Barbie et al., Nature, 462 (7269): 108-112).

In other embodiments, provided herein is a kit for determining or monitoring effectiveness of a treatment in a subject having long COVID comprising an agent for obtaining a first expression level of a biomarker in a first sample from the subject, wherein the biomarker is a CAP selected from the group consisting of IKZF2, IKZF3, ABCE1, BACH2, CD3D and HSPA8; and an agent for obtaining a second expression level of the biomarker in a second sample obtained from the subject after administering a treatment to the subject. In some embodiments, the kit further comprises an instruction on how to determine or monitor effectiveness of a treatment in a subject having long COVID. In some embodiments, the kit provides an instruction on determining the effectiveness of the treatment based on the comparison of the first expression level with the second expression level. In some embodiments, the kit provides an instruction on determining that the treatment is effective if the second expression level is lower than the first expression level. In some embodiments, the kit further comprises a tool for obtaining a sample from a subject. In some embodiments, the kit is for determining or monitoring effectiveness of an immunomodulatory drug (IMiD) in a subject having long COVID. In some embodiments, the kit is for determining or monitoring effectiveness of a CRBN modulator or a compound capable of binding and/or inducing conformational change to CRBN in a subject having long COVID. In some embodiments, the kit is for determining or monitoring effectiveness of an agent that depletes B cells (e.g., an anti-CD20 antibody, e.g., rituximab) in a subject having long COVID.

In certain embodiments, the kit further comprises instructions for determining or adjusting (e.g., increasing) a dose of the treatment or administering a different long COVID treatment to the subject if the second expression level is not lower than the first expression level.

In yet other embodiments, provided herein is a kit for screening a compound for effectiveness in treating long COVID comprising an agent for obtaining a first level of a biomarker in a sample, wherein the biomarker is a CAP selected from the group consisting of IKZF2, IKZF3, ABCE1, BACH2, CD3D and HSPA8; and an agent for obtaining a second level of the biomarker in the sample after administering the compound to the sample. In some embodiments, the kit further comprises an instruction on how to select a compound for treating long COVID. In some embodiments, the kit provides an instruction on selecting the compound if the second level is lower than the first level. In some embodiments, the kit further comprises a tool for obtaining a sample. In some embodiments, the kit is for screening an immunomodulatory drug (IMiD) for effectiveness in treating long COVID. In some embodiments, the kit is for screening a CRBN modulator or a compound capable of binding and/or inducing conformational change to CRBN for effectiveness in treating long COVID. In some embodiments, the kit is for screening an agent that depletes B cells (e.g., an anti-CD20 antibody, e.g., rituximab) for effectiveness in treating long COVID.

In some embodiments, the kit provided herein comprises instructions for determining that the treatment is effective if the second expression level is at least 10%, at least 20%, at least 30%, at least 40%, at least 50%, at least 60%, at least 70%, at least 80%, or at least 90% lower than the first expression level. Based on the comparison of the first expression level and the second expression level, a different treatment or a different dosing regimen may be administered to the subject in the subsequent treatment cycle(s).

In some embodiments, the treatment disclosed herein comprises an immunomodulatory drug (IMiD) as disclosed in Section 5.2. In some embodiments, the treatment comprises a CRBN modulator or a compound capable of binding and/or inducing conformational change to CRBN as disclosed in Section 5.2. In some embodiments, the treatment comprises an agent that depletes B cells (e.g., an anti-CD20 antibody, e.g., rituximab) as disclosed in Section 5.2.

In some embodiments of various kits provided herein, the kit comprises agents for determining/measuring the expression levels of two or more biomarkers, three or more biomarkers, four or more biomarkers, five or more biomarkers, or all biomarkers selected from the group consisting of IKZF2, IKZF3, ABCE1, BACH2, CD3D and HSPA8.

In some embodiments, the kit comprises agents for determining/measuring the expression levels of IKZF2 and at least one, two, three or four of IKZF3, ABCE1, BACH2, CD3D and HSPA8. In some embodiments, the kit comprises agents for determining/measuring the expression levels of IKZF3 and at least one, two, three or four of IKZF2, ABCE1, BACH2, CD3D and HSPA8. In some embodiments, the kit comprises agents for determining/measuring the expression levels of ABCE1 and at least one, two, three or four of IKZF2, IKZF3, BACH2, CD3D and HSPA8. In some embodiments, the kit comprises agents for determining/measuring the expression levels of BACH2 and at least one, two, three or four of IKZF2, IKZF3, ABCE1, CD3D and HSPA8. In some embodiments, the kit comprises agents for determining/measuring the expression levels of CD3D and at least one, two, three or four of IKZF2, IKZF3, ABCE1, BACH2, and HSPA8. In some embodiments, the kit comprises agents for determining/measuring the expression levels of HSPA8 and at least one, two, three or four of IKZF2, IKZF3, ABCE1, BACH2, and CD3D. In some embodiments, the kit comprises agents for determining/measuring the expression levels of all biomarkers in the group consisting of IKZF2, IKZF3, ABCE1, BACH2, CD3D and HSPA8. In some embodiments, the kit comprises agents for determining/measuring the expression level of IKZF2. In some embodiments, the kit comprises agents for determining/measuring the expression level of IKZF3. In some embodiments, the kit comprises agents for determining/measuring the expression level of ABCE1. In some embodiments, the kit comprises agents for determining/measuring the expression level of BACH2. In some embodiments, the kit comprises agents for determining/measuring the expression level of CD3D. In some embodiments, the kit comprises agents for determining/measuring the expression level of HSPA8. In certain embodiments, the kit comprises agents for determining/measuring the expression levels of two or more biomarkers selected from the group consisting of IKZF2, IKZF3, ABCE1, BACH2, CD3D and HSPA8. In certain embodiments, the kit comprises agents for determining/measuring the expression levels of IKZF2, IKZF3, ABCE1, BACH2, CD3D and HSPA8. In certain embodiments, the kit comprises agents for determining/measuring the expression levels of IKZF2, IKZF3, ABCE1, BACH2, CD3D and HSPA8. In certain embodiments, the kit comprises agents for determining/measuring the expression levels of IKZF2, IKZF3, ABCE1, BACH2, CD3D and HSPA8. In certain embodiments, the kit comprises agents for determining/measuring the expression levels of IKZF2, IKZF3, ABCE1, BACH2, CD3D and HSPA8. In some embodiments, the kit comprises agents for determining/measuring the expression levels of IKZF2 and IKZF3. In some embodiments, the kit comprises agents for determining/measuring the expression levels of IKZF2 and ABCE1. In some embodiments, the kit comprises agents for determining/measuring the expression levels of IKZF2 and BACH2. In some embodiments, the kit comprises agents for determining/measuring the expression levels of IKZF2 and CD3D. In some embodiments, the kit comprises agents for determining/measuring the expression levels of IKZF2 and HSPA8. In some embodiments, the kit comprises agents for determining/measuring the expression levels of IKZF3 and ABCE1. In some embodiments, the kit comprises agents for determining/measuring the expression levels of IKZF3 and BACH2. In some embodiments, the kit comprises agents for determining/measuring the expression levels of IKZF3 and CD3D. In some embodiments, the kit comprises agents for determining/measuring the expression levels of IKZF3 and HSPA8. In some embodiments, the kit comprises agents for determining/measuring the expression levels of ABCE1 and BACH2. In some embodiments, the kit comprises agents for determining/measuring the expression levels of ABCE1 and CD3D. In some embodiments, the kit comprises agents for determining/measuring the expression levels of ABCE1 and HSPA8. In some embodiments, the kit comprises agents for determining/measuring the expression levels of BACH2 and CD3D. In some embodiments, the kit comprises agents for determining/measuring the expression levels of BACH2 and HSPA8. In some embodiments, the kit comprises agents for determining/measuring the expression levels of CD3D and HSPA8. In some embodiments, the kit comprises agents for determining/measuring the expression levels of IKZF2, IKZF3 and ABCE1. In some embodiments, the kit comprises agents for determining/measuring the expression levels of IKZF2, IKZF3 and BACH2. In some embodiments, the kit comprises agents for determining/measuring the expression levels of IKZF2, IKZF3 and CD3D. In some embodiments, the kit comprises agents for determining/measuring the expression levels of IKZF2, IKZF3 and HSPA8. In some embodiments, the kit comprises agents for determining/measuring the expression levels of IKZF2, ABCE1 and BACH2. In some embodiments, the kit comprises agents for determining/measuring the expression levels of IKZF2, ABCE1 and CD3D. In some embodiments, the kit comprises agents for determining/measuring the expression levels of IKZF2, ABCE1 and HSPA8. In some embodiments, the kit comprises agents for determining/measuring the expression levels of IKZF2, BACH2 and CD3D. In some embodiments, the kit comprises agents for determining/measuring the expression levels of IKZF2, BACH2 and HSPA8. In some embodiments, the kit comprises agents for determining/measuring the expression levels of IKZF2, CD3D and HSPA8. In some embodiments, the kit comprises agents for determining/measuring the expression levels of IKZF3, ABCE1 and BACH2. In some embodiments, the kit comprises agents for determining/measuring the expression levels of IKZF3, ABCE1 and CD3D. In some embodiments, the kit comprises agents for determining/measuring the expression levels of IKZF3, ABCE1 and HSPA8. In some embodiments, the kit comprises agents for determining/measuring the expression levels of IKZF3, BACH2 and CD3D. In some embodiments, the kit comprises agents for determining/measuring the expression levels of IKZF3, BACH2 and HSPA8. In some embodiments, the kit comprises agents for determining/measuring the expression levels of IKZF3, CD3D and HSPA8. In some embodiments, the kit comprises agents for determining/measuring the expression levels of ABCE1, BACH2 and CD3D. In some embodiments, the kit comprises agents for determining/measuring the expression levels of ABCE1, BACH2 and HSPA8. In some embodiments, the kit comprises agents for determining/measuring the expression levels of ABCE1, CD3D and HSPA8. In some embodiments, the kit comprises agents for determining/measuring the expression levels of BACH2, CD3D and HSPA8. In some embodiments, the kit comprises agents for determining/measuring the expression levels of ABCE1, BACH2, CD3D and HSPA8. In some embodiments, the kit comprises agents for determining/measuring the expression levels of IKZF3, BACH2, CD3D and HSPA8. In some embodiments, the kit comprises agents for determining/measuring the expression levels of IKZF3, ABCE1, CD3D and HSPA8. In some embodiments, the kit comprises agents for determining/measuring the expression levels of IKZF3, ABCE1, BACH2 and HSPA8. In some embodiments, the kit comprises agents for determining/measuring the expression levels of IKZF3, ABCE1, BACH2 and CD3D. In some embodiments, the kit comprises agents for determining/measuring the expression levels of IKZF2, BACH2, CD3D and HSPA8. In some embodiments, the kit comprises agents for determining/measuring the expression levels of IKZF2, ABCE1, CD3D and HSPA8. In some embodiments, the kit comprises agents for determining/measuring the expression levels of IKZF2, ABCE1, BACH2 and HSPA8. In some embodiments, the kit comprises agents for determining/measuring the expression levels of IKZF2, ABCE1, BACH2 and CD3D. In some embodiments, the kit comprises agents for determining/measuring the expression levels of IKZF2, IKZF3, CD3D and HSPA8. In some embodiments, the kit comprises agents for determining/measuring the expression levels of IKZF2, IKZF3, BACH2 and HSPA8. In some embodiments, the kit comprises agents for determining/measuring the expression levels of IKZF2, IKZF3, BACH2 and CD3D. In some embodiments, the kit comprises agents for determining/measuring the expression levels of IKZF2, IKZF3, ABCE1 and HSPA8. In some embodiments, the kit comprises agents for determining/measuring the expression levels of IKZF2, IKZF3, ABCE1 and CD3D. In some embodiments, the kit comprises agents for determining/measuring the expression levels of IKZF2, IKZF3, ABCE1 and BACH2. In some embodiments, the kit comprises agents for determining/measuring the expression levels of IKZF3, ABCE1, BACH2, CD3D and HSPA8. In some embodiments, the kit comprises agents for determining/measuring the expression levels of IKZF2, ABCE1, BACH2, CD3D and HSPA8. In some embodiments, the kit comprises agents for determining/measuring the expression levels of IKZF2, IKZF3, BACH2, CD3D and HSPA8. In other embodiments, the kit comprises agents for determining/measuring the expression levels of IKZF2, IKZF3, ABCE1, CD3D and HSPA8. In other embodiments, the kit comprises agents for determining/measuring the expression levels of IKZF2, IKZF3, ABCE1, BACH2 and HSPA8. In other embodiments, the kit comprises agents for determining/measuring the expression levels of IKZF2, IKZF3, ABCE1, BACH2 and CD3D. In some embodiments, the kit comprises agents for determining/measuring the expression levels of IKZF2, IKZF3, ABCE1, BACH2, CD3D and HSPA8.

In some embodiments, the kit provides a reference expression level for each biomarker when agents for multiple biomarkers are included in the kit. In other embodiments, the kit provides a reference composite score for multiple biomarkers.

In some embodiments, the kit comprises agents for determining the protein level. In other embodiments, the kit comprises agents for determining the mRNA level. In yet other embodiments, the kit comprises agents for determining the cDNA level.

In certain embodiments, provided herein is a kit for detecting the mRNA level of one or more biomarkers. In certain embodiments, the kit comprises one or more probes that bind specifically to the mRNAs of the one or more biomarkers. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more biomarkers. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit can employ, for example, a dipstick, a membrane, a chip, a disk, a test strip, a filter, a microsphere, a slide, a multi-well plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an organ, an organelle, a biological fluid, a blood sample, a urine sample, or a skin sample.

In another embodiment, the kit comprises a solid support, nucleic acids attached to the support, where the nucleic acids are complementary to at least 1, 2, 3, 4, 5, 6, 10, 20, 50, 100, 200, 350, or more bases of mRNA, and a means for detecting the expression of the mRNA in a biological sample.

In a specific embodiment, the kit comprises, in a container, a compound or a pharmaceutical composition thereof, and further comprises, in one or more containers, components for isolating RNA. In another specific embodiment, the kit comprises, in a container, a compound or a pharmaceutical composition, and further comprises, in one or more containers, components for conducting RT-PCR, qRT-PCR, deep sequencing, or microarray.

In certain embodiments, the kits provided herein employ means for detecting the expression of a biomarker by quantitative real-time PCR (qRT-PCR), microarray, flow cytometry, immunofluorescence, sequencing (e.g., Next Generation Sequencing or NGS). In other embodiments, the expression of the biomarker is measured by ELISA-based methodologies or other similar methods known in the art.

In another specific embodiment, the kit comprises, in a container, a compound or a pharmaceutical composition thereof, and further comprises, in one or more containers, components for isolating protein. In another specific embodiment, the pharmaceutical or assay kit comprises, in a container, a compound or a pharmaceutical composition, and further comprises, in one or more containers, components for conducting flow cytometry or ELISA.

In another aspect, provided herein are kits for measuring biomarkers that supply the materials necessary to measure the abundance of one or more gene products of the biomarkers or a subset of the biomarkers (e.g., one, two, three, four, five, or more biomarkers) provided herein.

Such kits may comprise materials and reagents required for measuring RNA or protein. In some embodiments, such kits include microarrays, wherein the microarray is comprised of oligonucleotides and/or DNA and/or RNA fragments which hybridize to one or more gene products of the biomarkers or a subset of the biomarkers provided herein, or any combination thereof. In some embodiments, such kits may include primers for PCR of either the RNA product or the cDNA copy of the RNA product of the biomarkers or a subset of the biomarkers, or both. In some embodiments, such kits may include primers for PCR as well as probes for qPCR. In some embodiments, such kits may include multiple primers and multiple probes, wherein some of the probes have different fluorophores so as to permit simultaneously measuring multiple gene products of the biomarkers or a subset of the biomarkers provided herein. In some embodiments, such kits may further include materials and reagents for creating cDNA from RNA. In some embodiments, such kits may include antibodies specific for the protein products of the biomarkers or a subset of the biomarkers provided herein. Such kits may additionally comprise materials and reagents for isolating RNA and/or proteins from a biological sample. In addition, such kits may include materials and reagents for synthesizing cDNA from RNA isolated from a biological sample. In some embodiments, such kits may include a computer program product embedded on computer readable media for performing various functions according to the present method. In some embodiments, the kits may include a computer program product embedded on a computer readable media along with instructions.

In some embodiments, such kits measure the expression of one or more nucleic acid products of the biomarkers or a subset of the biomarkers provided herein. In accordance with this embodiment, the kits may comprise materials and reagents that are necessary for measuring the expression of particular nucleic acid products of the biomarkers or a subset of the biomarkers provided herein. For example, a microarray or RT-PCR kit may be produced for a specific condition and contain only those reagents and materials necessary for measuring the levels of specific RNA transcript products of the biomarkers or a subset of the biomarkers provided herein, to predict whether a patient is clinically sensitive to a compound. Alternatively, in some embodiments, the kits can comprise materials and reagents necessary for measuring the expression of particular nucleic acid products of genes other than the biomarkers provided herein. For example, in certain embodiments, the kits comprise materials and reagents necessary for measuring the expression levels of 1 genes, 2 genes, 3 genes, 4 genes, 5 genes, 6 genes, 7 genes, 8 genes, 9 genes, 10 genes, 15 genes, 20 genes, 25 genes, 30 genes, 35 genes, 40 genes, 45 genes, 50 genes, or more of the genes of the biomarkers provided herein, in addition to reagents and materials necessary for measuring the expression levels of at least 1 gene, at least 2 genes, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 35 genes, at least 40 genes, at least 45 genes, at least 50 genes, or more genes other than the biomarkers provided herein. In other embodiments, the kits contain reagents and materials necessary for measuring the expression levels of at least 1 genes, at least 2 genes, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 35 genes, at least 40 genes, at least 45 genes, at least 50 genes, or more of the biomarkers provided herein, and 1 gene, 2 genes, 3 genes, 4 genes, 5 genes, 10 genes, 15 genes, 20 genes, 25 genes, 30 genes, 35 genes, 40 genes, 45 genes, 50 genes, 55 genes, 60 genes, 65 genes, 70 genes, 75 genes, 80 genes, 85 genes, 90 genes, 95 genes, 100 genes, 125 genes, 150 genes, 175 genes, 200 genes, 225 genes, 250 genes, 300 genes, 350 genes, 400 genes, 450 genes, or more genes that are not the biomarkers provided herein. In certain embodiments, the kits contain reagents and materials necessary for measuring the expression levels of at least 1 genes, at least 2 genes, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 35 genes, at least 40 genes, at least 45 genes, at least 50 genes, or more of the genes of the biomarkers provided herein, and 1-10 genes, 1-100 genes, 1-150 genes, 1-200 genes, 1-300 genes, 1-400 genes, 1-500 genes, 1-1000 genes, 25-100 genes, 25-200 genes, 25-300 genes, 25-400 genes, 25-500 genes, 25-1000 genes, 100-150 genes, 100-200 genes, 100-300 genes, 100-400 genes, 100-500 genes, 100-1000 genes or 500-1000 genes that are not the biomarkers provided herein.

For nucleic acid microarray kits, the kits generally comprise probes attached to a solid support surface. In one such embodiment, probes can be either oligonucleotides or longer probes including probes ranging from 150 nucleotides to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from performing the assay. In a specific embodiment, the kits comprise instructions for predicting whether a patient is clinically sensitive to a compound. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own suitable container.

In certain embodiments, a nucleic acid microarray kit comprises materials and reagents necessary for measuring the expression levels of 1 gene, 2 genes, 3 genes, 4 genes, 5 genes, 6 genes, 7 genes, 8 genes, 9 genes, 10 genes, 15 genes, 20 genes, 25 genes, 30 genes, 35 genes, 40 genes, 45 genes, 50 genes, or more of the genes of the biomarkers provided herein, or a combination thereof, in addition to reagents and materials necessary for measuring the expression levels of at least 1 gene, at least 2 genes, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 35 genes, at least 40 genes, at least 45 genes, at least 50 genes, or more genes other than those of the biomarkers provided herein. In other embodiments, a nucleic acid microarray kit contains reagents and materials necessary for measuring the expression levels of at least 1 gene, at least 2 genes, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 35 genes, at least 40 genes, at least 45 genes, at least 50 genes, or more of the genes of the biomarkers provided herein, or any combination thereof, and 1 gene, 2 genes, 3 genes, 4 genes, 5 genes, 10 genes, 15 genes, 20 genes, 25 genes, 30 genes, 35 genes, 40 genes, 45 genes, 50 genes, 55 genes, 60 genes, 65 genes, 70 genes, 75 genes, 80 genes, 85 genes, 90 genes, 95 genes, 100 genes, 125 genes, 150 genes, 175 genes, 200 genes, 225 genes, 250 genes, 300 genes, 350 genes, 400 genes, 450 genes, or more genes that are not of the biomarkers provided herein. In another embodiment, a nucleic acid microarray kit contains reagents and materials necessary for measuring the expression levels of at least 1 gene, at least 2 genes, at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 30 genes, at least 35 genes, at least 40 genes, at least 45 genes, at least 50 genes, or more of the genes of the biomarkers provided herein, or any combination thereof, and 1-10 genes, 1-100 genes, 1-150 genes, 1-200 genes, 1-300 genes, 1-400 genes, 1-500 genes, 1-1000 genes, 25-100 genes, 25-200 genes, 25-300 genes, 25-400 genes, 25-500 genes, 25-1000 genes, 100-150 genes, 100-200 genes, 100-300 genes, 100-400 genes, 100-500 genes, 100-1000 genes, or 500-1000 genes that are not of the biomarkers provided herein.

For quantitative PCR, the kits generally comprise pre-selected primers specific for particular nucleic acid sequences. The quantitative PCR kits may also comprise enzymes suitable for amplifying nucleic acids (e.g., polymerases such as Taq polymerase), deoxynucleotides, and buffers needed for amplification reaction. The quantitative PCR kits may also comprise probes specific for the nucleic acid sequences associated with or indicative of a condition. The probes may or may not be labeled with a fluorophore. The probes may or may not be labeled with a quencher molecule. In some embodiments, the quantitative PCR kits also comprise components suitable for reverse-transcribing RNA, including enzymes (e.g., reverse transcriptase such as AMV, MMLV, and the like) and primers for reverse transcription along with deoxynucleotides and buffers needed for reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the reaction and methods for interpreting and analyzing the data resulting from performing the reaction. In a specific embodiment, the kits contain instructions for predicting whether a patient is clinically sensitive to a compound.

For antibody-based kits, the kit can comprise, for example: (1) a first antibody (which may or may not be attached to a solid support) that binds to a peptide, polypeptide or protein of interest; and, optionally, (2) a second, different antibody that binds to either the first antibody or the peptide, polypeptide, or protein, and is conjugated to a detectable label (e.g., a fluorescent label, radioactive isotope, or enzyme). In a specific embodiment, the peptide, polypeptide, or protein of interest is associated with or indicative of a condition (e.g., a disease). The antibody-based kits may also comprise beads for conducting immunoprecipitation. Each component of the antibody-based kits is generally in its own suitable container. Thus, these kits generally comprise distinct containers suitable for each antibody and reagent. Further, the antibody-based kits may comprise instructions for performing the assay and methods for interpreting and analyzing the data resulting from performing the assay. In a specific embodiment, the kits contain instructions for predicting whether a patient is clinically sensitive to a compound.

In one embodiment, a kit provided herein comprises a compound provided herein, or a pharmaceutically acceptable salt, solvate, stereoisomer, isotopologue, prodrug, hydrate, co-crystal, clathrate, or a polymorph thereof. Kits may further comprise additional active agents, including but not limited to those disclosed herein. Kits provided herein may further comprise devices that are used to administer the active ingredients. Examples of such devices include, but are not limited to, syringes, drip bags, patches, and inhalers.

In some embodiments, kits may further comprise cells or blood for transplantation, as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to, water for injection USP; aqueous vehicles (such as, but not limited to, sodium chloride injection, Ringer's injection, dextrose injection, dextrose and sodium chloride injection, and lactated Ringer's injection); water-miscible vehicles (such as, but not limited to, ethyl alcohol, polyethylene glycol, and polypropylene glycol); and non-aqueous vehicles (such as, but not limited to, corn oil, cottonseed oil, peanut oil, sesame oil, ethyl oleate, isopropyl myristate, and benzyl benzoate).

In certain embodiments of the methods and kits provided herein, solid phase supports are used for purifying proteins, labeling samples, or carrying out the solid phase assays. Examples of solid phases suitable for carrying out the methods disclosed herein include beads, particles, colloids, single surfaces, tubes, multi-well plates, microtiter plates, slides, membranes, gels, and electrodes. When the solid phase is a particulate material (e.g., a bead), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support, and the like, are also contemplated in relation to any of the various methods and/or kits provided herein.

In another aspect, provided herein is a kit for performing a method provided herein. In some embodiments, provided herein is a kit for identifying a subject having Chronic Fatigue Syndrome (CFS) or verifying CFS in a subject, comprising obtaining a level of a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; and identifying or verifying the subject as having CFS if the level of the urine organic acid in the sample is different from a reference level of the urine organic acid. In some embodiments, the kit further comprises an instruction for identifying or verifying the subject as having CFS if the level of the organic acid is lower than a reference level of the organic acid. In some embodiments, the kit (for example, in the instruction) provides the reference level of the organic acid. In some embodiments, the kit further comprises a tool for obtaining a sample from a subject.

In some embodiments, provided herein is a kit for determining severity of CFS in a subject, comprising obtaining a level of a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; comparing the level of the urine organic acid in the sample with a reference level of the urine organic acid; and determining the severity of CFS in the subject based on the comparison of the level of the urine organic in the sample to the reference level of the urine organic acid. In some embodiments, the kit further comprises an instruction on how to determine severity of CFS. In some embodiments, the kit (for example, in the instruction) provides the reference level of the organic acid. In some embodiments, the kit provides an instruction on comparing the level of the organic acid with the level of the organic acid and determining the severity of CFS based on the comparison. In some embodiments, the kit further comprises a tool for obtaining a sample from a subject.

In some embodiments, provided herein is a kit for identifying a subject who is likely or not likely to be responsive to a treatment of CFS or predicting the responsiveness of a subject to a treatment of CFS, comprising obtaining a level a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; and identifying or predicting the subject as being likely to be responsive to a treatment of CFS if the level of the urine organic acid in the sample is different from a reference level of the urine organic acid. In some embodiments, the kit further comprises an instruction on how to determine severity of CFS. In some embodiments, the kit (for example, in the instruction) provides the reference level of the organic acid. In some embodiments, the kit provides an instruction on comparing the level of the organic acid with the level of the organic acid and determining the severity of CFS based on the comparison. In some embodiments, the kit further comprises a tool for obtaining a sample from a subject.

In some embodiments, provided herein is a kit for selectively treating a subject having or suspected of having CFS with a treatment, comprising obtaining a level a urine organic acid in a sample from the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; identifying or predicting the subject as being likely to be responsive to a treatment of CFS if the level of the urine organic acid in the sample is different from a reference level of the urine organic acid; and administering the treatment to the subject identified or predicted to be likely to be responsive to the treatment. In some embodiments, the kit further comprises an instruction on how to determine severity of CFS. In some embodiments, the kit (for example, in the instruction) provides the reference level of the organic acid. In some embodiments, the kit provides an instruction on comparing the level of the organic acid with the level of the organic acid and determining the severity of CFS based on the comparison. In some embodiments, the kit further comprises a tool for obtaining a sample from a subject.

In some embodiments provided herein is a kit for determining or monitoring effectiveness of a treatment in a subject having CFS comprising obtaining a first level of a urine organic acid in a first sample from the subject before administering the treatment to the subject, wherein the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; administering the treatment to the subject, and obtaining a second level of the urine organic acid in a second sample obtained from the subject after administering the treatment to the subject; and determining the effectiveness of the treatment based on the comparison of the first level with the second level. In some embodiments, the kit further comprises an instruction on how to determine severity of CFS. In some embodiments, the kit (for example, in the instruction) provides the reference level of the organic acid. In some embodiments, the kit provides an instruction on comparing the level of the organic acid with the level of the organic acid and determining the severity of CFS based on the comparison. In some embodiments, the kit further comprises a tool for obtaining a sample from a subject.

In some embodiments, provided herein is a kit for screening a compound for effectiveness in treating CFS, the method comprising obtaining a first level of a urine organic acid in a sample, wherein (i) the urine organic acid is selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; (ii) xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid; and/or (iii) the urine organic acid is vanilmandelic acid; administering the compound to the sample; obtaining a second level of the urine organic acid in the sample after administering the compound to the sample; comparing the first level with the second level; and selecting the compound if the second level is different from the first level, wherein the compound is selected if the second level is higher than the first level. In some embodiments, the kit further comprises an instruction on how to determine severity of CFS. In some embodiments, the kit (for example, in the instruction) provides the reference level of the organic acid. In some embodiments, the kit provides an instruction on comparing the level of the organic acid with the level of the organic acid and determining the severity of CFS based on the comparison. In some embodiments, the kit further comprises a tool for obtaining a sample from a subject.

In some embodiments of various kits provided herein, the kit comprises agents for determining/measuring the levels of two or more organic acids, three or more organic acids, four or more organic acids, five or more organic acids, or all organic acids selected from the group consisting of hippuric acid, 3-hyroxypropionic acid, alpha-ketoisocaproic acid, alpha-ketoisovaleric acid, alpha-keto-beta-methylvaleric acid, alpha-hydroxybutyric acid, glycolic acid, pyruvic acid, citramalic acid, lactic acid, alpha-ketoadipic acid, citric acid, malic acid, kynurenic acid, xanthurenic acid, isovalerylglycine, 3-hydroxyisovaleric acid, isocitric acid, cis-aconitic acid, pyroglutamic acid, vanilmandelic acid, methylmalonic acid, and glyceric acid; (ii) xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid.

In some embodiments of the various kits provided herein, the reference expression level of the organic acid provided in the kit is a predetermined expression level of the organic acid from a public database. In some embodiments of the various kits provided herein, the reference level of the organic acid provided in the kit is a level of the organic acid in a healthy subject or in a subject who does not have CFS. In some embodiments, the reference level of the organic acid is a level of the organic acid in a subject having moderate CFS. In some embodiments of the various kits provided herein, the reference level of the organic acid provided in the kit is a level of the organic acid determined based on a cohort of subjects (e.g., a cohort of healthy subjects, a cohort of subjects not having CFS, or a cohort of subjects having moderate CFS). In some embodiments, the reference level of the organic acid is a median or a mean level of the organic acid of the levels of the organic acid in a cohort of subjects (e.g., a cohort of healthy subjects, a cohort of subjects not having CFS, or a cohort of subjects having moderate CFS).

In some embodiments, the kit provides a reference level for each organic acid when agents for multiple organic acids are included in the kit. In other embodiments, the kit provides a reference composite score for multiple organic acids.

In some embodiments, the kit comprises agents for determining the level of an organic acid or multiple organic acids. In some embodiments, the kit comprises instructions for using mass spectrometry (MS) to determine the level of an organic acid or multiple organic acids. In some embodiments, the MS comprises liquid chromatography-tandem mass spectrometry (LC MS/MS), liquid chromatography-mass spectrometry (LC-MS), multiple reaction monitoring (MRM), selected reaction monitoring (SRM), affinity-capture MS (AC-MS), matrix-assisted laser desorption/ionization time-of-flight (MALDI-TOF) MS, MALDI-TOF post-source-decay (PSD), MALDI-TOF/TOF, surface-enhanced laser desorption/ionization time-of-flight mass spectrometry (SELDI-TOF) MS, electrospray ionization mass spectrometry (ESI-MS), ESI-MS/MS, ESI-MS/(MS)n (n is an integer greater than zero), ESI 3D or linear (2D) ion trap MS, ESI triple quadrupole MS, ESI quadrupole orthogonal TOF (Q-TOF), ESI Fourier transform MS systems, desorption/ionization on silicon (DIOS), secondary ion mass spectrometry (SIMS), atmospheric pressure chemical ionization mass spectrometry (APCI-MS), APCI-MS/MS, APCI-(MS)n, ion mobility spectrometry (IMS), inductively coupled plasma mass spectrometry (ICP-MS)atmospheric pressure photoionization mass spectrometry (APPI-MS), APPI-MS/MS, and APPI-(MS)n.

### 6. EMBODIMENTS

The present disclosure includes the following non-limiting embodiments:
1. A method of identifying a subject having Chronic Fatigue Syndrome (CFS) or verifying CFS in a subject, the method comprising:
   (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; and
   (b) identifying or verifying the subject as having CFS if the expression level of the biomarker is higher than a reference expression level of the biomarker. 2. The method of embodiment 1, wherein the subject has reported chronic debilitating fatigue, unrefreshing sleep, mental and/or physical pain, neurological and cognitive impairment, and/or autoimmunity or immunodeficiencies. 3. A method of determining severity of CFS in a subject, the method comprising:
   (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D;
   (b) comparing the expression level of the biomarker with a reference expression level of the biomarker; and
   (c) determining the severity of CFS in the subject based on the comparison in step (b).
4. The method of embodiment 3, wherein the severity of CFS is determined to be severe if the expression level of the biomarker is higher than the reference expression level of the biomarker, optionally wherein the reference expression level of the biomarker is the expression level of the biomarker in a subject having mild CFS or a cohort of subjects having mild CFS.
5. A method of identifying a subject who is likely or not likely to be responsive to a treatment of CFS or predicting the responsiveness of a subject to a treatment of CFS, the method comprising:
   (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; and
   (b) identifying or predicting the subject as being likely to be responsive to the treatment if the expression level of the biomarker is higher than a reference expression level of the biomarker.
6. The method of embodiment 5, wherein the method further comprises administering the treatment to the subject identified or predicted to be likely to be responsive to the treatment.
7. A method of selectively treating a subject having or suspected of having CFS with a treatment, the method comprising:
   (a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D;
   (b) identifying or predicting the subject as being likely to be responsive to the treatment of CFS if the expression level of the biomarker is higher than a reference expression level of the biomarker; and
   (c) administering the treatment to the subject identified or predicted to be likely to be responsive to the treatment.
8. The method of any one of embodiments 1-7, wherein the reference expression level of the biomarker is a predetermined expression level of the biomarker.
9. The method of any one of embodiments 1-8, wherein the reference expression level of the biomarker is the expression level of the biomarker in a subject who does not have CFS or a cohort of subjects not having CFS.
10. The method of any one of embodiments 1-8, wherein the reference expression level of the biomarker is the expression level of the biomarker in a healthy subject or a cohort of healthy subjects.
11. The method of any one of embodiments 1-8, wherein the reference expression level of the biomarker is the expression level of the biomarker in a subject having mild CFS or a cohort of subjects having mild CFS.
12. The method of any one of embodiments 1-11, wherein the biomarker is HSPA8 or ABCE1, optionally wherein the reference expression level of the biomarker is the expression level of the biomarker in a healthy subject or a subject who does not have CFS, or a cohort of healthy subjects or subjects not having CFS.
13. The method of any one of embodiments 1-12, wherein the method comprises determining the expression levels of two, three, four, five, or all biomarkers selected from the group consisting of HSPA8, ABCE1, IKZF2, IKZF3, BACH2 and CD3D.
14. The method of embodiment 13, wherein the method comprises determining the expression levels of:
   (i) IKZF2 and at least one, two, three or four of IKZF3, ABCE1, BACH2, CD3D and HSPA8;
   (ii) IKZF3 and at least one, two, three or four of IKZF2, ABCE1, BACH2, CD3D and HSPA8;
   (iii) ABCE1 and at least one, two, three or four of IKZF2, IKZF3, BACH2, CD3D and HSPA8;
   (iv) BACH2 and at least one, two, three or four of IKZF2, IKZF3, ABCE1, CD3D and HSPA8;
   (v) CD3D and at least one, two, three or four of IKZF2, IKZF3, ABCE1, BACH2, and HSPA8; or
   (vi) HSPA8 and at least one, two, three or four of IKZF2, IKZF3, ABCE1, BACH2, and CD3D.
15. The method of embodiment 13, wherein the method comprises determining the expression levels IKZF2, IKZF3, ABCE1, BACH2, CD3D and HSPA8. 16. The method of any one of embodiments 13-15, wherein the method comprises comparing the expression level of each of the biomarkers with their respective reference expression level.
17. The method of any one of embodiments 13-15, wherein the method comprises obtaining a composite score based on the expression levels of the biomarkers and comparing the composite score with a reference score derived from the reference expression levels of the biomarkers.
18. A method of determining or monitoring effectiveness of a treatment in a subject having CFS, the method comprising:
   (a) determining a first expression level of a biomarker in a first sample obtained from the subject before administering the treatment to the subject, wherein the biomarker is a cereblon (CRBN)-associated protein (CAP) selected from the group consisting of HSPA8, ABCE1, IKZF2, IKZF3, BACH2 and CD3D;
   (b) administering the treatment to the subject;
   (c) determining a second expression level of the biomarker in a second sample obtained from the subject after administering the treatment to the subject; and
   (d) determining the effectiveness of the treatment based on the comparison of the first expression level with the second expression level.
19. The method of embodiment 18, wherein the method comprises determining that the treatment is effective if the second expression level is lower than the first expression level.
20. The method of embodiment 18 or 19, wherein the method further comprises determining or adjusting a dose of the treatment to the subject.
21. A method of screening a treatment for effectiveness in treating CFS, the method comprising:
   (a) determining a first expression level of a biomarker in a sample before administering the treatment to the sample, wherein the biomarker is a cereblon (CRBN)-associated protein (CAP) selected from the group consisting of HSPA8, ABCE1, IKZF2, IKZF3, BACH2 and CD3D;
   (b) administering the treatment to the sample;
   (c) determining a second expression level of the biomarker in the sample after administering the treatment to the sample;
   (d) comparing the first expression level with the second expression level; and
   (e) selecting the treatment if the second expression level is lower than the first expression level.
22. The method of any one of embodiments 18-21, wherein the biomarker is HSPA8 or ABCE1.
23. The method of any one of embodiments 18-22, wherein the method comprises determining the first and second expression levels of two, three, four, five, or all biomarkers selected from the group consisting of HSPA8, ABCE1, IKZF2, IKZF3, BACH2 and CD3D.
24. The method of embodiment 23, wherein the method comprises determining the first and second expression levels of:
   (i) IKZF2 and at least one, two, three or four of IKZF3, ABCE1, BACH2, CD3D and HSPA8;
   (ii) IKZF3 and at least one, two, three or four of IKZF2, ABCE1, BACH2, CD3D and HSPA8;
   (iii) ABCE1 and at least one, two, three or four of IKZF2, IKZF3, BACH2, CD3D and HSPA8;
   (iv) BACH2 and at least one, two, three or four of IKZF2, IKZF3, ABCE1, CD3D and HSPA8;
   (v) CD3D and at least one, two, three or four of IKZF2, IKZF3, ABCE1, BACH2, and HSPA8; or
   (vi) HSPA8 and at least one, two, three or four of IKZF2, IKZF3, ABCE1, BACH2, and CD3D.
25. The method of embodiment 23, wherein the method comprises determining the first and second expression levels of all biomarkers in the group consisting of HSPA8, ABCE1, IKZF2, IKZF3, BACH2 and CD3D.
26. The method of any one of embodiments 23-25, wherein the method comprises comparing the first expression level of each of the biomarkers with their respective second expression level.
27. The method of any one of embodiments 23-25, wherein the method comprises obtaining a first composite score based on the first expression levels of the biomarkers and a second composite score based on the second expression level of the biomarkers, and comparing the first composite score with the second composite score.
28. The method of any one of embodiments 5-27, wherein the treatment comprises an immunomodulatory drug (IMiD).
29. The method of any one of embodiments 5-27, wherein the treatment comprises a celebron (CRBN) modulator or a compound capable of binding and/or inducing conformational change to CRBN.
30. The method of any one of embodiments 5-27, wherein the treatment comprises an agent that depletes B cells.
31. The method of any one of embodiments 1-30, wherein the CFS is associated with an autoimmune disease or a viral infection.
32. A method of identifying a subject having long COVID or verifying long COVID in a subject, the method comprising:
   (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and
   (b) identifying or verifying the subject as having long COVID if the expression level of the biomarker is higher than a reference expression level of the biomarker.
33. A method of identifying a subject who is likely or not likely to be responsive to a treatment of long COVID or predicting the responsiveness of a subject to a treatment of long COVID, the method comprising:
   (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and
   (b) identifying or predicting the subject as being likely to be responsive to the treatment if the expression level of the biomarker is higher than a reference expression level of the biomarker.
34. The method of embodiment 33, wherein the method further comprises administering the treatment to the subject identified or predicted to be likely to be responsive to the treatment.
35. A method of selectively treating a subject having or suspected of having long COVID with a treatment, the method comprising:
   (a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D;
   (b) identifying or predicting the subject as being likely to be responsive to a treatment of long COVID if the expression level of the biomarker is higher than a reference expression level of the biomarker; and
   (c) administering the treatment to the subject identified or predicted to be likely to be responsive to the treatment.
36. The method of any one of embodiments 32-35, wherein the reference expression level of the biomarker is a predetermined expression level of the biomarker.
37. The method of any one of embodiments 32-35, wherein the reference expression level of the biomarker is the expression level of the biomarker in a subject who does not have long COVID or a cohort of subjects not having long COVID.
38. The method of any one of embodiments 32-35, wherein the reference expression level of the biomarker is the expression level of the biomarker in a healthy subject or a cohort of healthy subjects.
39. The method of any one of embodiments 32-35, wherein the reference expression level of the biomarker is the expression level of the biomarker in a subject having acute COVID or a cohort of subjects having acute COVID.
40. The method of any one of embodiments 32-39, wherein the biomarker is HSPA8 or IKZF3, optionally wherein the reference expression level of the biomarker is the expression level of the biomarker in a healthy subject or a subject who does not have long COVID, or a cohort of healthy subjects or subjects not having long COVID.
41. The method of any one of embodiments 32-39, wherein the method comprises determining the expression levels of two, three, four, five, or all biomarkers selected from the group consisting of HSPA8, IKZF3, ABCE1, IKZF2, BACH2 and CD3D.
42. The method of embodiment 41, wherein the method comprises determining the expression levels of:
   (i) IKZF2 and at least one, two, three or four of IKZF3, ABCE1, BACH2, CD3D and HSPA8;
   (ii) IKZF3 and at least one, two, three or four of IKZF2, ABCE1, BACH2, CD3D and HSPA8;
   (iii) ABCE1 and at least one, two, three or four of IKZF2, IKZF3, BACH2, CD3D and HSPA8;
   (iv) BACH2 and at least one, two, three or four of IKZF2, IKZF3, ABCE1, CD3D and HSPA8;
   (v) CD3D and at least one, two, three or four of IKZF2, IKZF3, ABCE1, BACH2, and HSPA8; or
   (vi) HSPA8 and at least one, two, three or four of IKZF2, IKZF3, ABCE1, BACH2, and CD3D.
43. The method of embodiment 41, wherein the method comprises determining the expression levels HSPA8, IKZF3, ABCE1, IKZF2, BACH2 and CD3D.
44. The method of any one of embodiments 41-43, wherein the method comprises comparing the expression level of each of the biomarkers with their respective reference expression level.
45. The method of any one of embodiments 41-43, wherein the method comprises obtaining a composite score based on the expression levels of the biomarkers and comparing the composite score with a reference score derived from the reference expression levels of the biomarkers.
46. A method of determining or monitoring effectiveness of a treatment in a subject having long COVID, the method comprising:
   (a) determining a first expression level of a biomarker in a first sample obtained from the subject before administering the treatment to the subject, wherein the biomarker is a cereblon (CRBN)-associated protein (CAP) selected from the group consisting of HSPA8, IKZF3, ABCE1, IKZF2, BACH2 and CD3D;
   (b) administering the treatment to the subject;
   (c) determining a second expression level of the biomarker in a second sample obtained from the subject after administering the treatment to the subject; and
   (d) determining the effectiveness of the treatment based on the comparison of the first expression level with the second expression level.
47. The method of embodiment 46, wherein the method comprises determining that the treatment is effective if the second expression level is lower than the first expression level.
48. The method of embodiment 46 or 47, wherein the method further comprises determining or adjusting a dose of the treatment to the subject.
49. A method of screening a treatment for effectiveness in treating long COVID, the method comprising:
   (a) determining a first expression level of a biomarker in a sample before administering the treatment to the sample, wherein the biomarker is a cereblon (CRBN)-associated protein (CAP) selected from the group consisting of HSPA8, IKZF3, ABCE1, IKZF2, BACH2 and CD3D;
   (b) administering the treatment to the sample;
   (c) determining a second expression level of the biomarker in the sample after administering the treatment to the sample;
   (d) comparing the first expression level with the second expression level; and
   (e) selecting the treatment if the second expression level is lower than the first expression level.
50. The method of any one of embodiments 46-49, wherein the biomarker is HSPA8 or IKZF3. 51. The method of any one of embodiments 46-50, wherein the method comprises determining the first and second expression levels of two, three, four, five, or all biomarkers selected from the group consisting of HSPA8, IKZF3, ABCE1, IKZF2, BACH2 and CD3D.
52. The method of embodiment 51, wherein the method comprises determining the first and second expression levels of:
   (i) IKZF2 and at least one, two, three or four of IKZF3, ABCE1, BACH2, CD3D and HSPA8;
   (ii) IKZF3 and at least one, two, three or four of IKZF2, ABCE1, BACH2, CD3D and HSPA8;
   (iii) ABCE1 and at least one, two, three or four of IKZF2, IKZF3, BACH2, CD3D and HSPA8;
   (iv) BACH2 and at least one, two, three or four of IKZF2, IKZF3, ABCE1, CD3D and HSPA8;
   (v) CD3D and at least one, two, three or four of IKZF2, IKZF3, ABCE1, BACH2, and HSPA8; or
   (vi) HSPA8 and at least one, two, three or four of IKZF2, IKZF3, ABCE1, BACH2, and CD3D.
53. The method of embodiment 51, wherein the method comprises determining the first and second expression levels of all biomarkers in the group consisting of HSPA8, IKZF3, ABCE1, IKZF2, BACH2 and CD3D.
54. The method of any one of embodiments 51-53, wherein the method comprises comparing the first expression level of each of the biomarkers with their respective second expression level.
55. The method of any one of embodiments 51-53, wherein the method comprises obtaining a first composite score based on the first expression levels of the biomarkers and a second composite score based on the second expression level of the biomarkers, and comparing the first composite score with the second composite score.
56. The method of any one of embodiments 33-55, wherein the treatment comprises an immunomodulatory drug (IMiD).
57. The method of any one of embodiments 33-55, wherein the treatment comprises a CRBN modulator or a compound capable of binding and/or inducing conformational change to CRBN.
58. The method of any one of embodiments 33-55, wherein the treatment comprises an agent that depletes B cells.
59. The method of any one of embodiments 32-58, wherein the subject has had Coronavirus Disease 2019 (COVID-19).
60. The method of any one of embodiments 1-59, wherein the expression level of the biomarker is determined by measuring the mRNA level of the biomarker.
61. The method of embodiment 60, wherein the mRNA level is determined by using quantitative reverse-transcriptase PCR (RT-qPCR), microarray, Northern blot or RNA sequencing.
62. The method of any one of embodiments 1-59, wherein the expression level of the biomarker is determined by measuring the protein level of the biomarker.
63. The method of embodiment 62, wherein the protein level of the biomarker is determined by using mass spectrometry (MS), liquid chromatography-tandem mass spectrometry (LC MS/MS), immunoassay, flow cytometry, immunohistochemistry, western blot, or enzyme-linked immunosorbent assay (ELISA).
64. A kit for performing the method of any one of embodiments 1-63, the kit comprising an agent for determining the expression level of at least one biomarkers selected from the group consisting of HSPA8, IKZF3, ABCE1, IKZF2, BACH2 and CD3D.
65. The kit of embodiment 64, wherein the kit further comprises a tool for obtaining the sample.
66. The kit of embodiment 64 or 65, wherein the kit further comprises an instruction on interpreting the determined expression level.
67. The kit of any one of embodiments 64-66, wherein the kit further comprises the reference expression level of the biomarker.

### 7. EXAMPLES

The examples below are carried out using standard techniques, which are well known and routine to those of skill in the art, except where otherwise described in detail. The examples are intended to be merely illustrative.

### 7.1. Methods

### 7.1.1 Study Design

This study was divided into three phases: Startup, Conduct, and Testing & Analysis. During the Startup phase, a protocol was written to allow for both site-based and direct to patient (D2P) study implementation. The study originally aimed to collect samples at a single timepoint from 100 CFS patients and 100 healthy control participants, and samples from a second timepoint from 10% of each group to assess sampling repeatability. During the Conduct phase, subjects were recruited, qualified, consented, characterized, and sampled per the study protocol. During the Testing & Analysis phase, whole blood fingerstick samples were tested using the autoimmune profile (AIP) test at DxTerity (Rancho Dominguez, CA). A portion of the whole blood samples was extracted to yield DNA which was tested for viral infection using a real time PCR method at Coppe (Waukesha, WI). The urine samples were tested for organic acid profiles at Genova (Asheville, North Carolina).

### 7.1.2 Recruitment of Subjects

Participants were recruited directly through online advertising, patient advocates and advocacy groups, registries, clinical sites, and physician referral. Participants (male and female age 18 or older at the time of consent) completed a single study collection event consisting of one Micro Collection Device (MCD) for fingerstick blood and one first morning void urine collected from home. About 20% of each group (ME/CFS and Control) completed a second study collection event approximately 3 to 4 weeks after the initial study collection to assess sampling repeatability. For the Disease Group, patients must have been either diagnosed with ME/CFS by their physicians, or met CFS definitions (per 2015 IOM diagnostic criteria for ME/CFS definition) (Clayton, E. W., Beyond myalgic encephalomyelitis/chronic fatigue syndrome: an IOM report on redefining an illness. JAMA, 2015. 313(11): p. 1101-2) as indicated on the qualification questionnaire. For the Control Group, participants were eligible if they did not have history of fatigue and did not meet the ME/CFS definition (Clayton, E. W., Beyond myalgic encephalomyelitis/chronic fatigue syndrome: an IOM report on redefining an illness. JAMA, 2015. 313(11): p. 1101-2). Case and control recruitment were handled by DxTerity Diagnostics. A total of 166 ME/CFS cases and 83 controls were recruited, among them, 163 ME/CFS cases and 79 controls were included in this study.

### 7.1.3 Self-Reported Data Collection

Once enrolled in the study, participants were asked to self-report and provide the following information:
Demographics (Age, Gender (at birth), Height, Weight, Race and Ethnicity
History and status of illness (first collection time only)
   Year that ME/CFS symptoms started
   Do ME/CFS symptoms result in being house bound?
   Triggering Event (Not identifiable, bacterial infections/illness, viral infection/illness, physical trauma, emotional trauma or other)
   Diagnosis by Health Care Professional?
   Managed by a physician for ME/CFS condition?
   How well are conditions/symptoms managed?
Current medications and treatment history
   Antibiotics
   Chemotherapy
   Non-Steroidal Anti-Inflammatory Drugs
   Other Anti-Inflammatory Drugs
   Antihistamines
   Immunomodulatory drugs
   Dietary Supplements
Medical history
   Diagnosis with Autoimmune Disease
   Other conditions/diagnostics
Involvement in another study/clinical trial for ME/CFS Study experience

### 7.1.4 Sample Collection

Each participant received a DxCollect® MCD Fingerstick Kit and a Genova Diagnostics® Urine Collection Kit and was also asked to complete a study-related questionnaire. The MCD blood collection kit was used to facilitate the collection, stabilization, and shipping of a microsample (about 150 μL) of blood for non-clinical use. A urine sample was collected in a 15 mL tube pre-coated with a stabilizer containing a preservative. Upon mixing after urine collection, the urine sample was frozen down at −20° C. or less before shipment was made within 24 hours of collection.

### 7.1.5 Sample Analysis

AIP Testing: The MCD blood samples and Autoimmune Profiling (AIP) gene expression testing were carried out at DxTerity by using a method that combines an RNA-stabilizing buffer and the target-dependent chemical ligation of probes, followed by PCR amplification of the ligated probes to perform the quantitative analysis of multiple transcripts (Kim, C. H., et al., A novel technology for multiplex gene expression analysis directly from whole blood samples stabilized at ambient temperature using an RNA-stabilizing buffer. J Mol Diagn, 2015. 17(2): p. 118-27). Fifty-one genes on the AIP panel were separated by capillary electrophoresis and the gene expression levels were calculated relative to the geometric mean of 3 house-keeping control genes. The results were grouped into Modules which demonstrate correlation to immune pathways or therapeutic targets.

### Viral Infection Testing:

DNA from MCD blood samples were extracted at DxTerity. The extracted DNA samples were shipped to an independent lab (Coppe Laboratories, Waukesha, WI) for viral infection testing for HHV6-A, HHV6-B, HHV-7, EBV and CMV.

### Urine Organic Acid Testing:

The urine samples were tested per The Genova Diagnostics Organic Acids testing protocol. Organic acids are a broad class of compounds formed during fundamental metabolic processes in the body. Urinary amino acids were measured via GC/MS, LC/MS/MS and alkaline picrate. For P value and fold change, median organic acid values from CFS group and normal group were calculated by the Quantiles function using JMP software. One way ANOVA and Student's t-test were used to calculate Prob >F (pooled_Pval) and mean comparison. Fold change (FC) was calculated by dividing median_normal by median_CFS. Twelve out of 48 organic acids met a set criteria (i.e., a combination of p<0.05 and FC >1.4).

Similar analysis was completed between CFS_bedridden and CFS_non_bedridden subsets. There was one organic acid (Vanilmandelic Acid) out of 48 which has p<0.05.

### 7.1.6 Statistical AIP Data Analysis

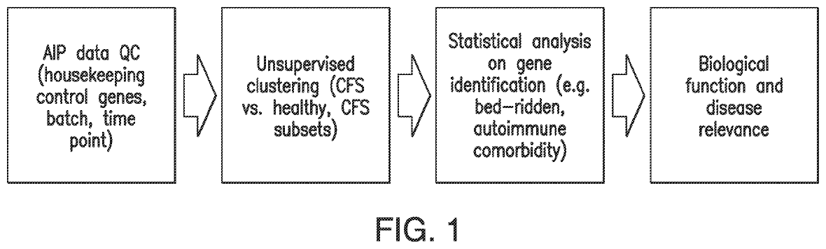

Computational data analyses were performed on the resulting data sets generated from each of the 3 tests to discover genes and metabolites associated with ME/CFS and/or sub-segmentation of ME/CFS patients based on disease severity and autoimmune disease comorbidities. The diagram in FIG. 1 depicts the data analysis workflow for AIP gene expression data.

### Normalization and Batch Quality Control:

In AIP testing, results from first timepoint (n=221) and second timepoint (n=45) were compared to assess biological concordance between replicate samples and confirm reproducibility of the proposed normalization procedure. AIP testing was performed in two batches depending on sample availability, with batch 1 (n=224) and batch 2 (n=43). Each batch contained a mixture of both timepoints.

Wilcoxon signed-rank tests were performed between replicates on both the raw and normalized relative florescent units (RFU) of three housekeeping genes (ACTB, GAPDH, TFRC). Coefficient of variation of housekeeping and PCR control levels were observed to ascertain their influence on possible variations in gene expression. Additionally, raw values were re-normalized by subtracting the raw RFU of each gene from the geometric mean reference expression of each patient for comparison to the initial results obtained from the DxTerity's AIP testing.

### Unsupervised Clustering and Obtaining Genes of Interest:

The Bioconductor package clusterExperiment (Risso, D., et al., clusterExperiment and RSEC: A Bioconductor package and framework for clustering of single-cell and other large gene expression datasets. PLoS Comput Biol, 2018. 14(9): p. e1006378) was used as a framework for resampling-based sequential ensemble clustering (RSEC) on the module genes. This workflow implements several clustering iterations over a range of both standard and user-defined tuning inputs such as k0 and alpha values, dimensionality reduction methods, and cluster sizes. A single consensus clustering was determined from several candidates and further merging of related hierarchical sister nodes is then performed.

Clustering was performed on the CFS cohort to reveal potential subgroups of interest with a required minimum consensus sample size of 5. The consensus yielded 6 major groups, denoted by m01-m06 (standard RSEC naming convention). The clinical and demographic composition of each cluster was tallied and assigned a category if 80% or more of the cluster contained that category type, i.e. if 80% of the cluster was female, the cluster was labeled as majority female.

Multivariable regression models were developed from the log 2 RFU of all 51 panel genes. Models controlled for demographic factors such as age, sex, and race. Clinical indications such as symptom duration, trigger event (yes/no), and whether the trigger event was based on prior viral or bacterial infection (yes/no), were also included in the additive model. Genes significantly (p<0.05) differentially expressed by each attribute were annotated within the rows of heatmaps developed from the scaled and centered log 2 RFU data to visually segment gene clusters of interest.

### Heatmaps of AIP Genes and Urine Organic Acid Data:

Heatmaps for ATP genes were derived from log 2 normalized RFU values and urine acid metabolites from mmol values normalized by group creatine levels. All heatmaps were scaled and centered prior to calculating Euclidean distances to represent dendrogram clusters.

### Regression Bootstrapping:

Regression models from genes which were borderline modulated between CFS bedridden and CFS non-bedridden patients (as determined based on prior multivariable regression models) underwent residual resampling to account for sample bias and potential noise in the dataset. Demographic and clinical confounders such as age, sex, prior infection, and symptom duration were controlled for. Coefficient confident intervals were reported from 2,000 resampling iterations.

### 7.1.8 COVID Analysis

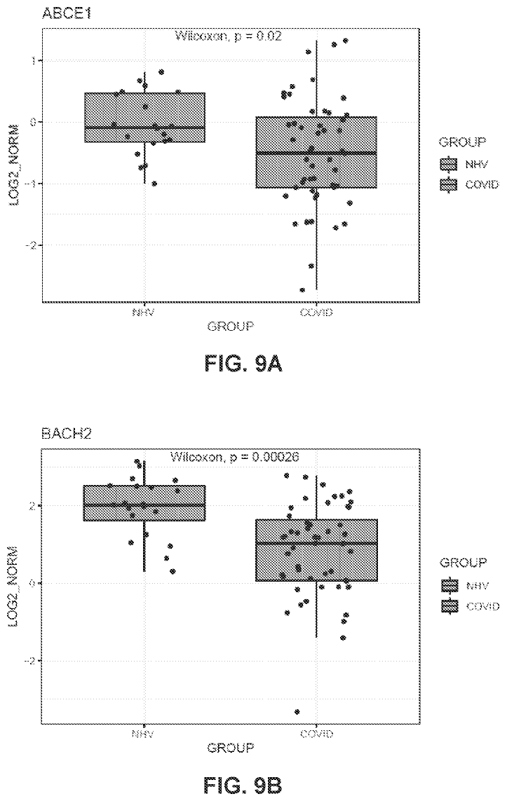

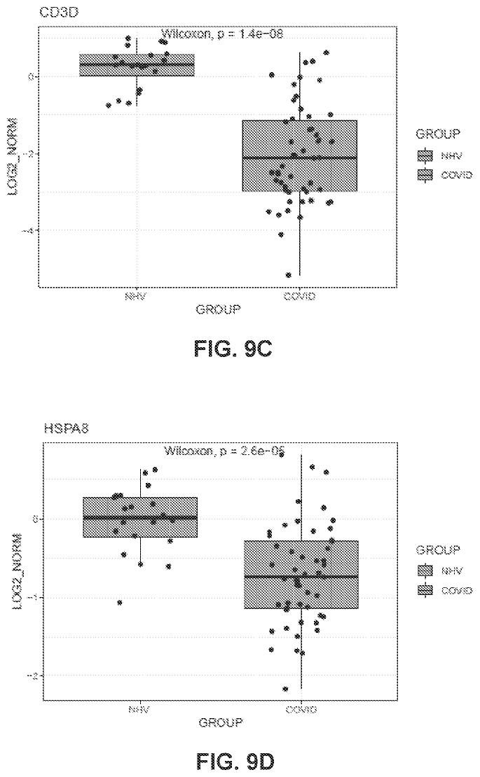

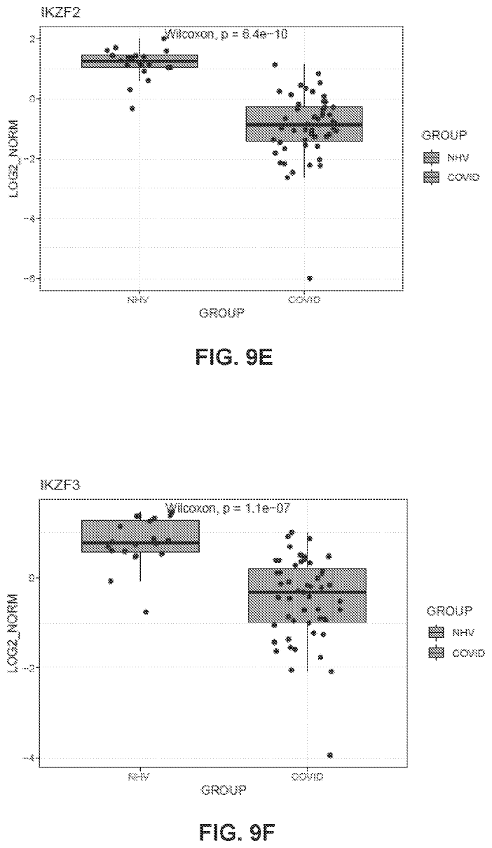

For analysis relating to acute COVID (see FIGS. 9A-9F), ATP gene expression of six markers in severe acute COVID patients and matching normal healthy volunteer (NHV) donors were obtained. Individual boxplots (ABCE1, BACH2, CD3D, HSPA8, IKZF2, IKZF3) were provided to compare gene expression between acute severe patients (COVID) and NHV subjects. Samples were RNA extracted from PAXgene blood from remnant Phase 2 Study of Intravenous Abatacept in the Treatment of Hospitalized COVID-19 Participants. Subjects included 51 severe acute COVID patients and 20 background matched NHV subjects. The COVID patients were confirmed SARS-CoV-2 infected and hospitalized (or in the ED awaiting hospitalization) with respiratory compromise as defined by requirement of oxygen supplementation but not requiring mechanical ventilation. No additional normalization was performed since there was no batch effect observed in the house keeping genes.

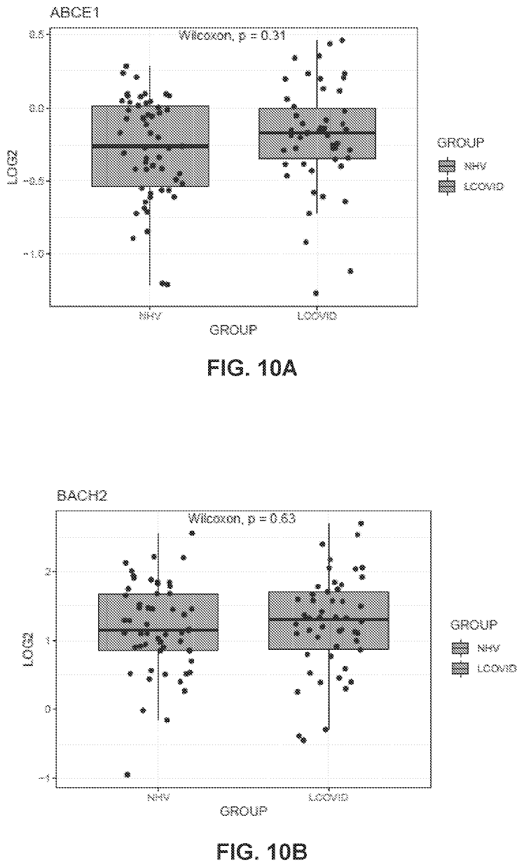

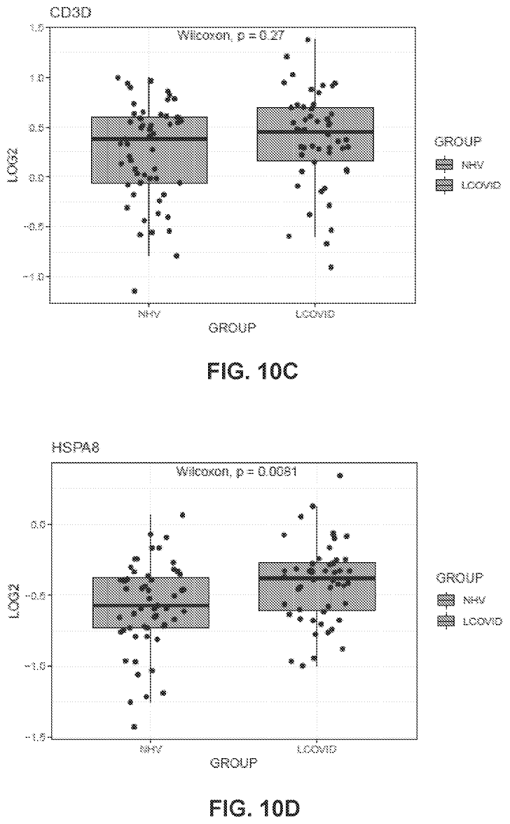

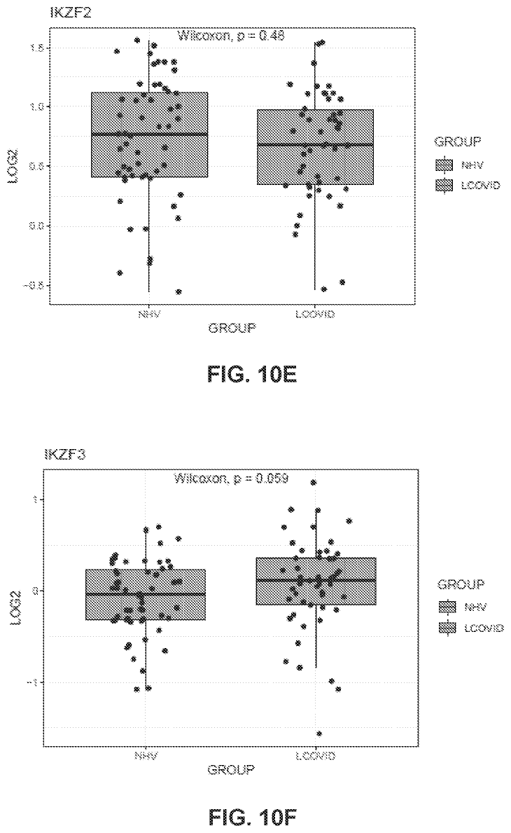

For analysis relating to long COVID (see FIGS. 10A-10F), samples were procured from commercial vendors. Samples included 50 Long COVID (all from MT Group) and 55 normal healthy volunteer (NHV) (50 from Discovery Life Sciences (DLS) and 5 from MT Group) donors. The Long COVID patients were confirmed diagnosis by a physician, and the Long COVID patients had Long COVID symptom(s) onset more than 6 months post SARS-CoV-2 infection (average 9 months). The Long COVID symptoms on the data of collection included but were not limited to: anosmia, body aches, cough, fatigue, headache, shortness of breath, respiratory symptoms, heart symptoms, digestive symptoms, or neurological symptoms. All subjects had no history of CFS, autoimmune disease, or any type of cancer. The NHV subject never had SARS-CoV-2 infection prior to sample collection. Gene expressions for ABCE1, BACH2, CD3D, HSPA8, IKZF2, and IKZF3 were determined in the subjects. The analysis used ComBat empirical-bayes batch adjustment by vendors/sites (DLS vs. MT group) using DLS NHV as the reference batch.

### 7.2. Results

### 7.2.1 Patient Characteristics

A total of 166 CFS and 83 healthy participants (controls) were recruited for the study. From the participants who completed the questionnaire, there were 125 females (75%) and 38 males (23%) in the ME/CFS group and 44 females (53%) and 34 males (41%) in the control group. The average age was at 50.8 in the ME/CFS participants and 44.8 in the controls (p=0.001). Average Body Mass Index (BMI) was very similar at 26.8 in the patients and 25.8 in the controls (p=0.522). Based on BMJ standard weight status categories, similar percentage of the ME/CFS participants and the controls fell into underweight, healthy weight, overweight and obesity categories with no statistical differences (p>0.05). Ninety-two percent (92%) of the ME/CFS participants and fifty-two (52%) of the controls were Caucasian. A much smaller percentage of both the groups were Asians, Hispanics, African Americans, and others. Table 1 below summarizes the cohort characteristics.

TABLE 1
Cohort characteristics

| | | | | | Mann-Whitney |
|---|---|---|---|---|---|
| | ME/CFS | | Controls | | U test |
| Total # Unique Subjects | 166 | | 83 | | NA |
| Age (years) | 50.8 ± 12.8 | | 44.8 ± 14.8 | | p < 0.05 |
| | Gender | | | | |
| Female | 125 | | 44 | | NA |
| Male | 38 | | 34 | | NA |
| BMI (kg/m2) | 26.8 ± 6.6 | | 25.8 ± 5.0 | | p = 0.52 |
| Underweight (<18.5) | 5 | (3%) | 2 | (2%) | NA |
| Healthy | 72 | (43%) | 34 | (41%) | p = 0.90 |
| Weight (18.5-24.9) | | | | | |
| Overweight (25.0-29.9) | 46 | (28%) | 28 | (34%) | p = 0.57 |
| Obesity (>30.0) | 40 | (24%) | 13 | (16%) | p = 0.27 |
| Unreported | 3 | (2%) | 6 | (7%) | NA |
| | Ethnicity | | | | |
| Asian | 2 | (1%) | 26 | (31%) | NA |
| Caucasian (White) | 152 | (92%) | 43 | (52%) | NA |
| Hispanic (non-White) | 4 | (2%) | 4 | (5%) | NA |
| Other | 5 | (3%) | 4 | (5%) | NA |
| African American/Black | 0 | (0%) | 1 | (1%) | NA |
| Unreported | 3 | (2%) | 5 | (6%) | NA |

Out of the 166 ME/CFS participants, ninety-four percent (94%) reported receiving diagnosis by a health care professional. Three percent (30%) were self-assessed based on descriptions provided in the questionnaire as they considered themselves to have the symptoms listed in the 2015 IOM diagnostic criteria for ME/CFS. The other three percent (3%) did not provide information, but they were placed in the ME/CFS group since these individuals identified as ME/CFS specific when enrolling in the study. Similarly, out of 83 normal participants, ninety-four percent (94%) of the subjects enrolled as healthy normal controls based on the exclusion criteria. Six percent (6%) did not provide information but were placed in the normal control group since these individuals identified as normal when enrolling.

Onset of CFS symptoms was reported by the participants. Duration of the CFS disease was calculated and reported: 170o of the ME/CFS participants reported to have CFS between 1-5 year, 18% between 6-10 years, 13% between 11-15 years, 12% between 16-20 years, 21% between 21-30 years, 16% between 31-63 years, 2% did not report.

Approximately half (49%) of the ME/CFS patients reported being house-bound or bed-ridden due to the symptoms of the disease. In this study, we considered house-bound or bed-ridden patients as having severe symptoms, and the rest as having mild to moderate symptoms (See Chang, C. J., et al., A Comprehensive Examination of Severely Ill ME/CFS Patients. Healthcare (Basel), 2021. 9(10)).

Seventy-six percent (76%) of the ME/CFS patients reported there was a triggering event before the onset of the disease. Among them, eighty percent (80%) reported an assignable viral or bacterial infection as the trigger, while twenty percent (20%) reported other physical and/or emotional traumatic events as the trigger. Among the viral and bacterial infections, organisms were listed in the questionnaire included: EBV, Mononucleosis, Flu, H. Pylori, Pneumonia, Strep Throat and COVID. In addition, thirty percent (30%) of the ME/CFS patients have or have had autoimmune diseases (e.g. Hashimoto's thyroiditis, Fibromyalgia, IBD, POTS, Grave's Disease, Crohn's disease, Psoriasis, Rheumatoid arthritis and Sjogren's syndrome). Thirteen percent (13%) have or have had various cancers. Five percent (5%) have or have had kidney diseases. Eighty-five (85%) of the CFS patients were taking medications (currently or within the last 4 weeks), including Antibiotics, Antihistamine, Immunomodulatory, Non-Steroidal Anti-Inflammatory drugs, comparing to 36% of the normal controls listed similar medications (not listed).

Table 2 below summarizes the above described patient history.

TABLE 2
Summary of Patient History

| | |
|---|---|
| ME/CFS | Controls |

Duration of CFS Disease (Year)

| | | | |
|---|---|---|---|
| 1-5 | 28 | (17%) | NA |
| 6-10 | 30 | (18%) | NA |
| 11-15 | 22 | (13%) | NA |
| 16-20 | 20 | (12%) | NA |
| 21-30 | 34 | (21%) | NA |
| 31-63 | 26 | (16%) | NA |
| Unreported | 4 | (2% | NA |

Diagnosed with ME/CFS or experiencing symptoms of ME/CFS

| | | | |
|---|---|---|---|
| Yes | 161 | (97%) | NA |

| | | | |
|---|---|---|---|
| No | NA | 78 | (94%) |

| | | | | |
|---|---|---|---|---|
| Unreported* | 5 | (3%) | 5 | (6%) |

Diagnosed with ME/CFS by a Health Care Professional

| | | | |
|---|---|---|---|
| Yes | 156 | (94%) | NA |
| No | 5 | (3%) | NA |
| Unreported* | 5 | (3%) | NA |

Severity of ME/CFS symptoms (house-bound or bed-ridden)

| | | | |
|---|---|---|---|
| Yes | 82 | (49%) | NA |
| No | 79 | (48%) | NA |
| Unreported* | 5 | (3%) | NA |

Triggering event leading to ME/CFS symptoms

| | | | |
|---|---|---|---|
| Yes | 126 | (76%) | NA |
| No | 35 | (21%) | NA |
| Unreported* | 5 | (3%) | NA |

Triggering events

| | | | |
|---|---|---|---|
| Viral, bacterial and/or | 101 | (80%) | NA |
| other unknown infection | | | |
| Physical and emotional | 25 | (20%) | NA |
| trauma and others | | | |

Diagnosed with any Autoimmune Diseases

| | | | | |
|---|---|---|---|---|
| Yes | 50 | (30%) | 8 | (10%) |
| No | 110 | (66%) | 69 | (83%) |
| Unreported* | 6 | (4%) | 6 | (7%) |

Diagnosed with any Cancer

| | | | | |
|---|---|---|---|---|
| Yes | 21 | (13%) | 2 | (2%) |
| No | 137 | (82%) | 71 | (86%) |
| Unreported* | 8 | (5%) | 10 | (12%) |

Diagnosed with Kidney Disease

| | | | | |
|---|---|---|---|---|
| Yes | 5 | (3%) | 0 | (0%) |
| No | 154 | (93%) | 73 | (88%) |
| Unreported* | 7 | (4%) | 10 | (12%) |

*Subjects did not provide information and were assigned to either CFS or Normal based on the enrollment link they chose for registering for the study.

### 7.2.2 Patient Centric Enrollment and Sampling: Direct to Patient (D2P)

A total of 300 participants were enrolled, with only 3 subjects having withdrawn from the study after the samples were collected. In the study, 267 valid AIP results and 272 urine results were included, after excluding QC fails and disqualified samples. Participant questionnaire was completed by 287/297 (96.30%) of the total participants. Ten subjects who did not complete the questionnaire were equally split between the CFS group and the control group. Results were compiled using a de-identified participant ID.

7.2.3 AIP Results Reveal Genes that are Up-Regulated in Bed-Ridden CFS Patients

MCD blood samples were collected from 163 CFS and 79 control participants at the first timepoint, and samples were also collected from 33 CFS and 15 control participants at the second timepoint (˜19% of each group). The second timepoint samples were collected voluntarily at approximately 1-2 months after the first time point. The purpose for testing at the second time point was to verify there was no sampling bias between the collections. Together, the AIP testing was completed for a total of 287 MCD samples (out of 240 participants including first and second timepoint collections). Out of the 287 tests, 267 tests (93.0%) passed QC acceptance.

To assess potential batch effects, both batches were combined and principal component analysis (PCA) was performed to visualize clusters of normalization genes and immune module genes separately. Replicate samples displayed a high Spearman's rank correlation coefficient (r=0.86, p<2.2e-16), but multivariable regression models revealed a downward shift in mean unique to timepoint. COMBAT (W. EVAN JOHNSON, C. L., Ariel Rabinovic, Adjusting for batch effects in microarray expression data using empirical Bayes methods. Biostatistics, 2007. 8(1): p. 118-127) empirical bayes standardization resolved this deviation, suggesting this relatively small batch effect was technical in nature.

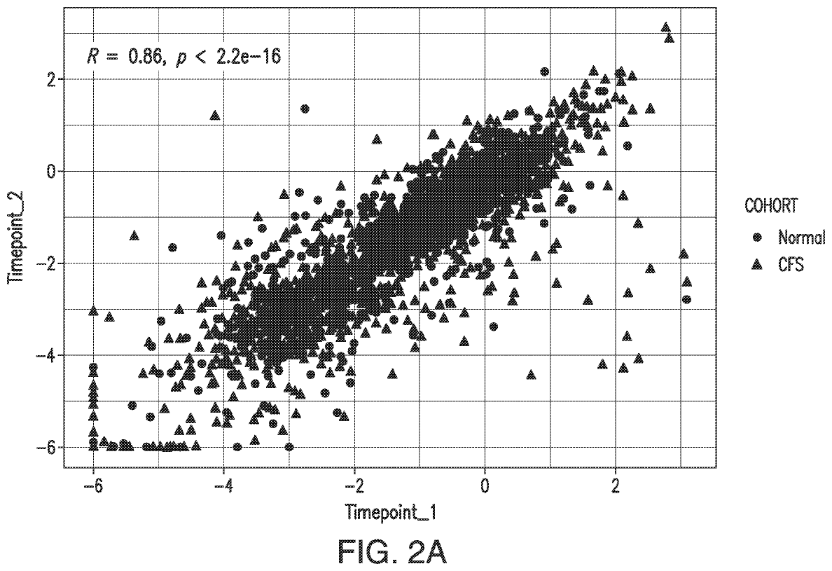

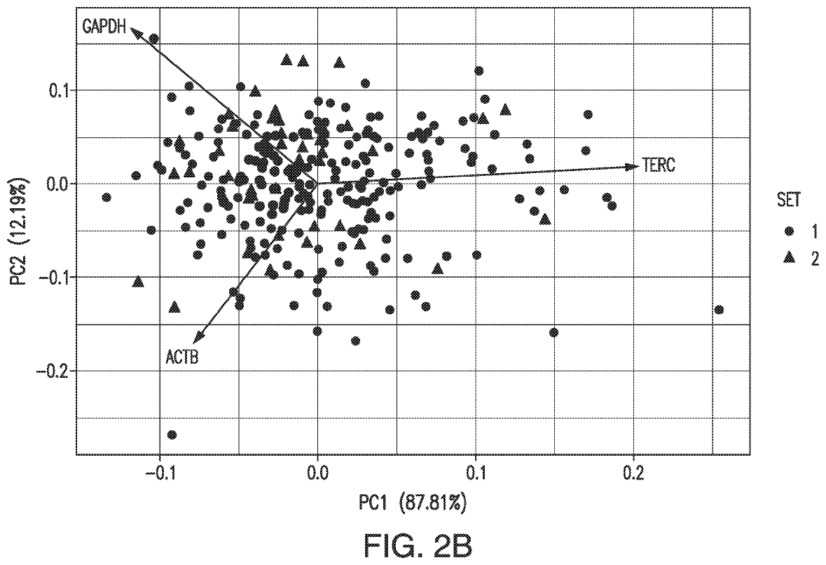

When examining concordance between the samples collected at two timepoints, the scatter plot in FIG. 2A showed a good correlation (R=0.86) between the timepoint 1 and 2 samples. In addition, the testing was carried out in 2 distinct batches. Since each gene signal (raw RFU) was normalized against the geometric mean of the 3 house-keeping genes (ACTB, GAPDH, TFRC), when examining the 3 house-keeping genes between the 2 batch data sets, the PCA plot showed that no observable difference in house-keeping genes by the 2 data sets, therefore there is no batch effect in sample testing (FIG. 2B). The AIP results in the subsequent analyses were based on the first time point (excluding repetitive samples from the same participants), in which all samples were collected from unique participants of either the CFS patients or the normal controls.

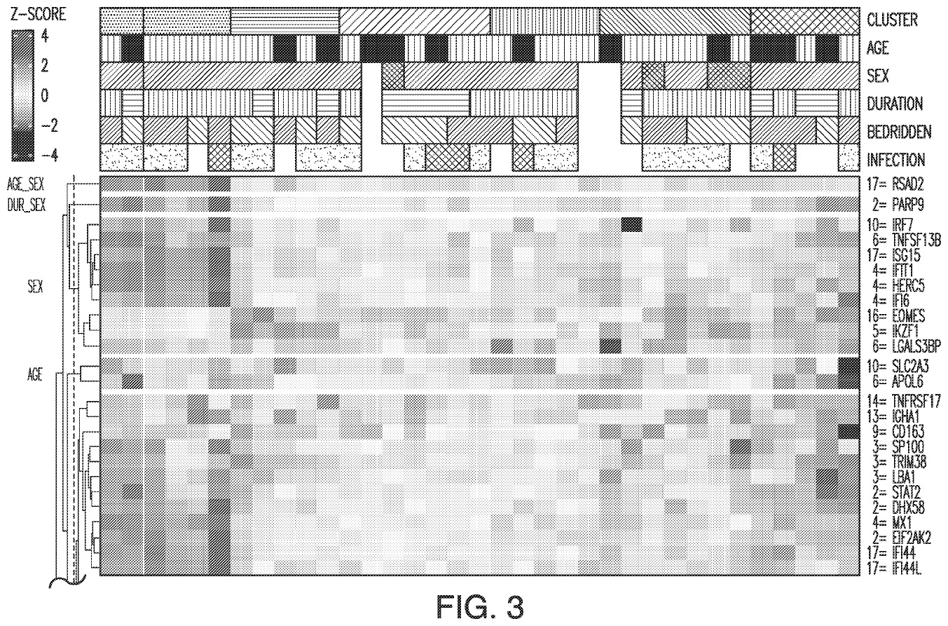

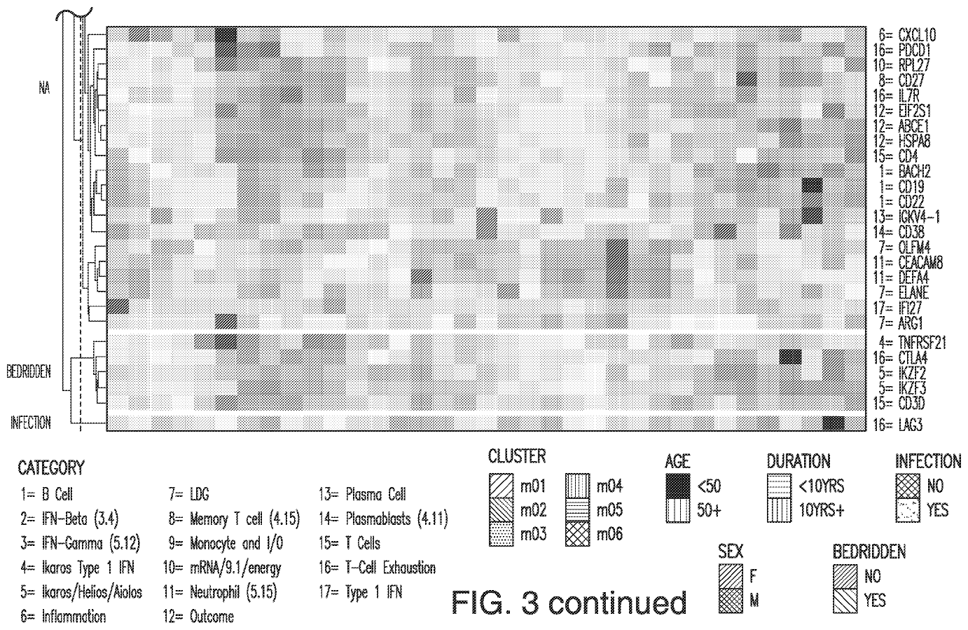

From the CFS group, unsupervised consensus clustering was conducted to reveal a heat map (FIG. 3) showing the relationship between genes significantly up- or down-regulated in patient subsets with certain demographic or clinical attributes, such as age, gender, disease duration, prior viral infection, and bed-ridden status. Six major clusters were unveiled after merging of several candidate clusters (labeled as m01-m06 in the heatmap). These groupings confirm categorical gene expression modulation also reported from prior regression testing, such as enrichment in IFIT1, ISG15, and IFI6 in female patients and higher expression of APOL6 and SLC2A3 in patients 55 years and older. In addition, there were also visible expression differences which may be explained by unreported factors of the patients.

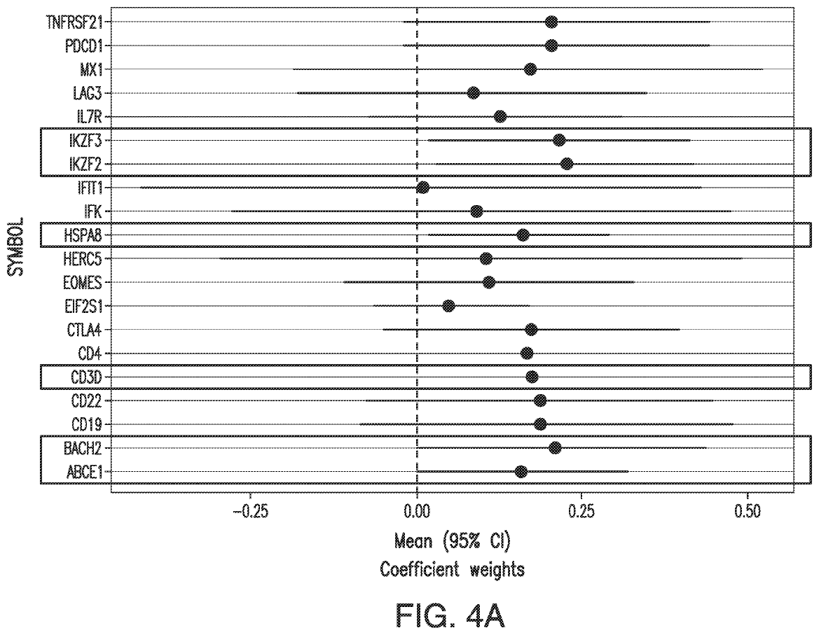

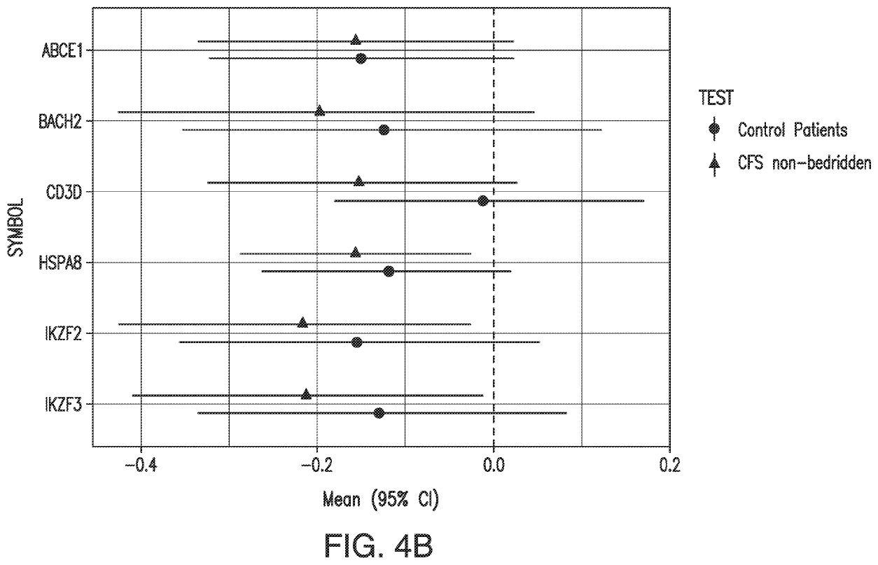

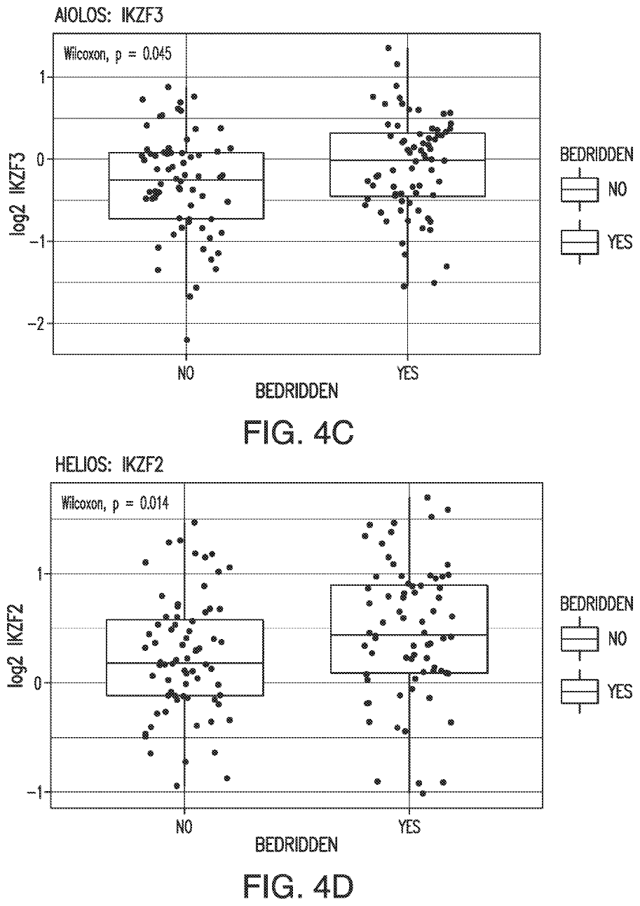

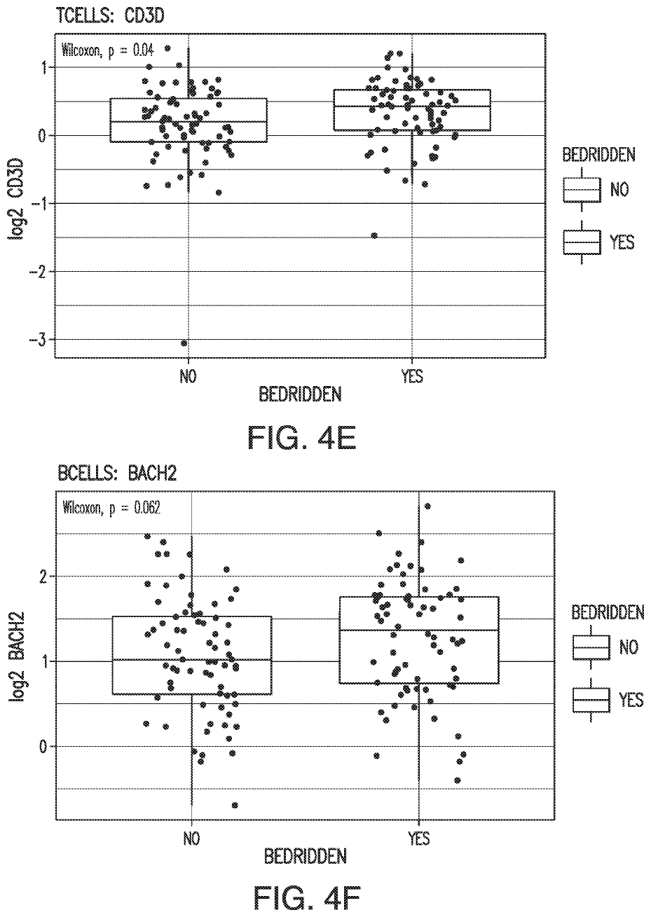

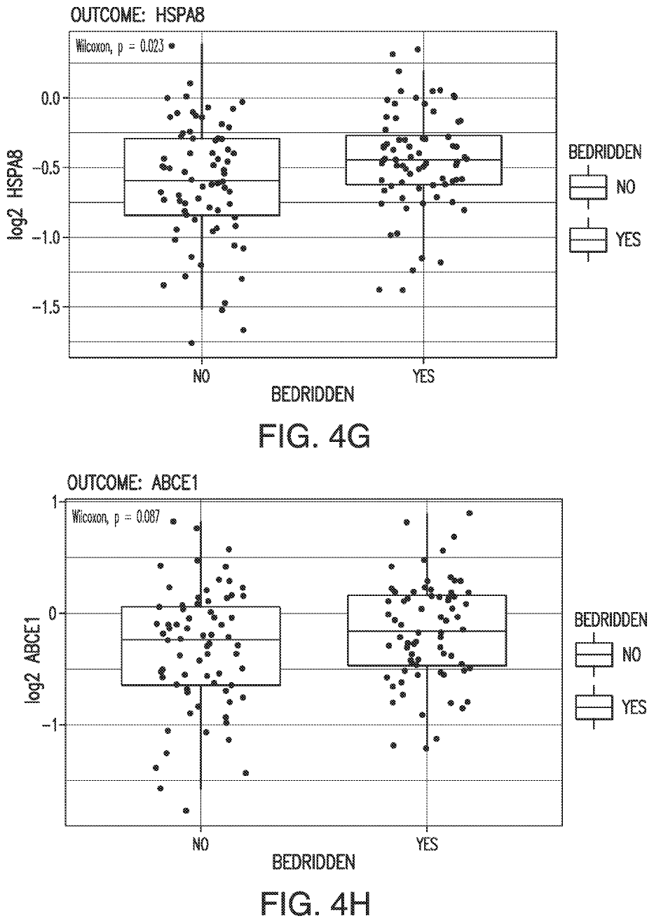

Next, genes that were differentially expressed between two subsets of the CFS patients were analyzed: self-reported bed-ridden patients (n=72) and self-reported non-bed-ridden patients (n=72). The bed-ridden patients were considered to have severe disease. Six genes were identified from the bootstrapping analysis, including IKZF2, IKZF3, ABCE1, BACH2, CD3D and HSPA8 (P<0.05; Table 3). A Forest Plot showed the coefficient means and 95% confidence intervals for each gene listed in Table 3, and FIG. 4A. Given that the sample composition disparity by sex in this dataset further widened by sub-setting among bedridden status, a bootstrapped multivariable model on bedridden status was performed controlling for age and sex. Of the 6 genes identified, the genes most associated with the bedridden status after controlling for age and sex were HSPA8, IKZF2, and IKZF3 (Table 4 and FIG. 4B). FIGS. 4C-4H show the expression levels of the 6 individual genes between the severe (bed-ridden) group and the mild (non-bed-ridden) group on the dataset prior to bootstrapping. Genes such as BACH2 and ABCE1 were not statistically significant at p<0.05 when testing on the full dataset but became borderline or statistically significant upon residual resampling, emphasizing that more vigorous testing can reduce sampling bias. The expression levels of IKZF2, IKZF3, ABCE1, BACH2, CD3D and HSPA8 were significantly higher in severe CFS patients (subject with CFS and bedridden) as compared to mild CFS patients (subject with CFS but not bedridden). The expression levels of ABCE1 and HSPA8 were borderline significantly higher in severe CFS patients (subject with CFS and bedridden) as compared to healthy patients. The expression levels of IKZF2, IKZF3, ABCE1, BACH2, CD3D and HSPA8 were not significantly higher in mild CFS patients (subject with CFS but not bedridden) as compared to healthy subjects.

As shown in Table 3, univariable bootstrapped analysis identified 6 genes differentiated by bedridden status in CFS patients (p<0.05, bold box). Regression models from 21 genes which were borderline modulated between CFS bedridden and CFS non-bedridden patients underwent residual resampling to account for sample bias and potential noise in the dataset. The gene symbol, coefficient means, lower and upper bound 95% confidence interval values, and confidence interval p-values are displayed.

As shown in Table 4, multivariable bootstrapped model of genes differentially expressed by bedridden status and controlled for age and gender identified 3 top genes. Multivariable regression models were developed from the log 2 RFU of all 51 panel genes. Bedridden CFS patients were used as the baseline factor for comparison between non-bedridden CFS and healthy patients The gene symbol, coefficient means, lower and upper bound 95% confidence interval values, and confidence interval p-values are displayed.

TABLE 4
Multivariable Bootstrapped Analysis

| SYMBOL | TEST | MEAN | LB | UB | ¹ PVAL | FDR |
|---|---|---|---|---|---|---|
| ABCE1 | BEDRIDDENNO | −0.1557521 | −0.3240262 | 0.0084173 | 0.071 | 0.118 |
| ABCE1 | BEDRIDDENHEALTHY | −0.1503423 | −0.3229919 | 0.0132119 | 0.070 | 0.118 |
| BACH2 | BEDRIDDENNO | −0.1969117 | −0.4160876 | 0.0344532 | 0.093 | 0.136 |
| BACH2 | BEDRIDDENHEALTHY | −0.1238034 | −0.3690103 | 0.1220105 | 0.295 | 0.295 |
| CD3D | BEDRIDDENNO | −0.1524633 | −0.3289913 | 0.0178588 | 0.079 | 0.131 |
| CD3D | BEDRIDDENHEALTHY | −0.0117965 | −0.1925623 | 0.1697525 | 0.901 | 0.901 |
| HSPA8 | BEDRIDDENNO | −0.1564088 | −0.2880166 | −0.0252872 | 0.023 | 0.056 |
| HSPA8 | BEDRIDDENHEALTHY | −0.1181249 | −0.2673568 | 0.0158163 | 0.086 | 0.143 |
| IKZF2 | BEDRIDDENNO | −0.2169488 | −0.4123106 | −0.0191753 | 0.033 | 0.081 |
| IKZF2 | BEDRIDDENHEALTHY | −0.1546637 | −0.3487566 | 0.0443747 | 0.126 | 0.209 |
| JKZF3 | BEDRIDDENNO | −0.2119117 | −0.4086699 | −0.0121177 | 0.041 | 0.203 |
| IKZF3 | BEDRIDDENHEALTHY | −0.1299734 | −0.2288182 | 0.0818228 | 0.205 | 0.513 |

¹ bootstrapped confidence interval p-value

7.2.4 AIP Results Reveal Genes that are Upregulated in CFS Severe Cases with Autoimmune Comorbidity

A subset of CFS patients also has an autoimmune etiology (Sotzny, F., et al., Myalgic Encephalitis/Chronic Fatigue Syndrome—Evidence for an autoimmune disease. Autoimmun Rev, 2018. 17(6): p. 601-609). In this study, 30% of CFS participants reported having one or more autoimmune diseases that co-exist with CFS. For these 44 patients, gene expression levels were compared of the 6 genes between the 22 bed-ridden and the 22 non-bed-ridden patients.

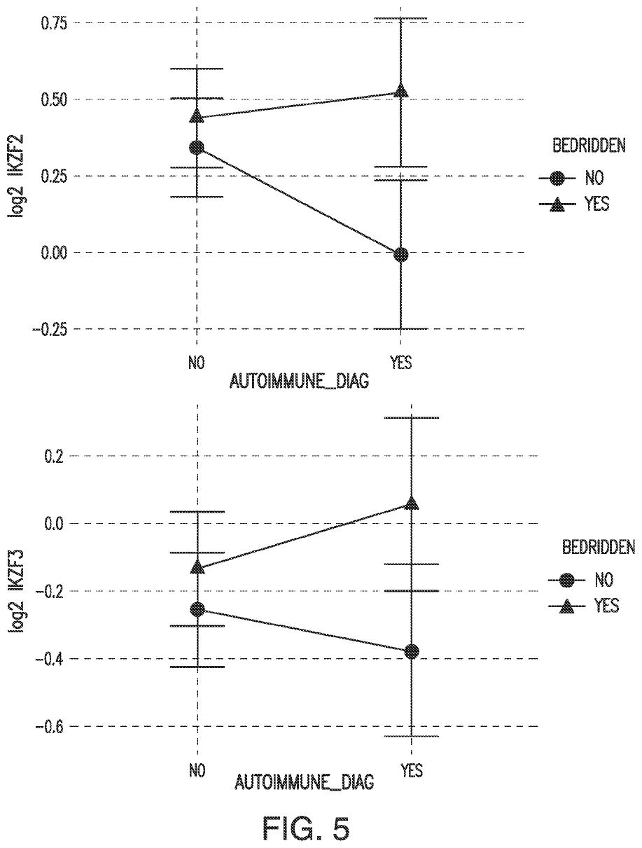

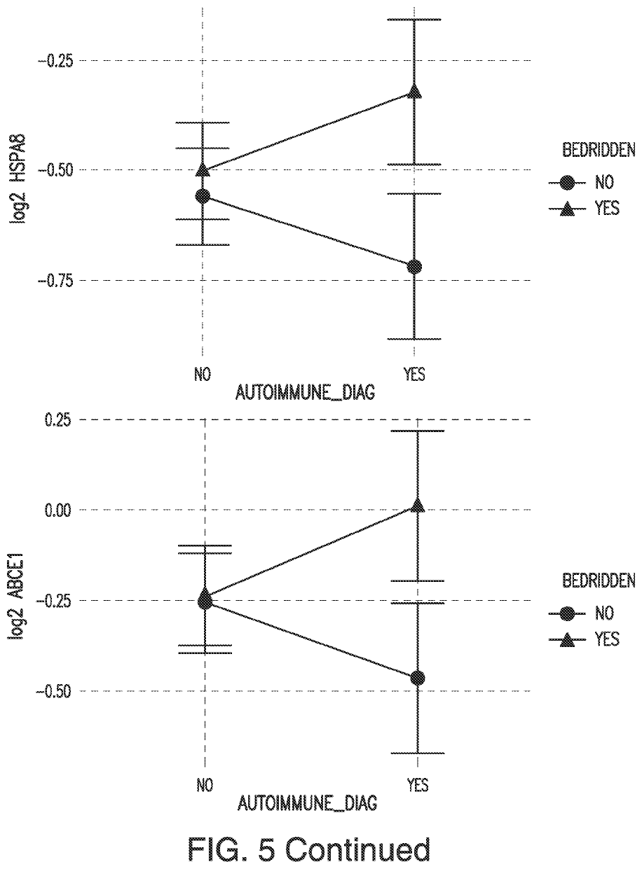

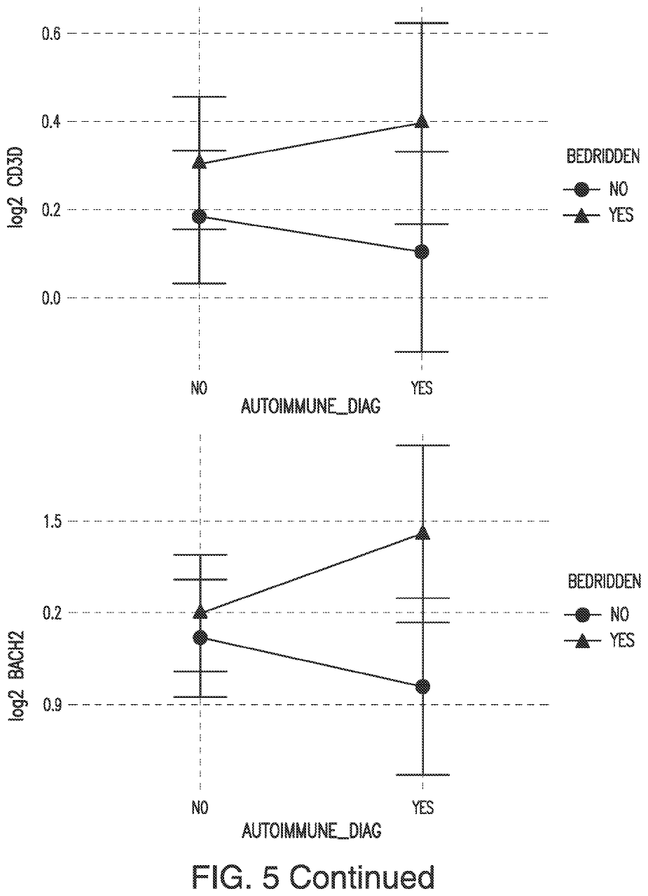

Each of the 6 genes exhibited a greater separation in this subset of CFS patients with other autoimmune diseases (FIG. 5), suggesting an additive effect of fatigue related gene expression in CFS and other autoimmune etiologies.

### 7.2.5 Urine Organic Acid Profiling Reveals Difference Between CFS and Normal Cohort

Profiles of urine organic acids can provide insight into key metabolic irregularities that relate to nutritional cofactor needs, digestive irregularities, cellular energy production, neurotransmitter metabolism, and detoxification. In this study, profiles of urinary organic acids (when using raw values of organic acid, i.e., no normalization against creatinine) showed statistically significant (p<0.05) differences between CFS patients and normal subjects in 23 organic acids (see FIGS. 6A-6F). These organic acids belong to six organic acid categories. Twenty-three out of forty-eight organic acids had p<0.05 (as indicated by the asterisks in FIG. 6A). Twelve organic acids had median fold change of >1.4 (decreased in CFS) and p<0.05: xanthurenic acid, glycolic acid, pyruvic acid, hippuric acid, isovalerylglycine, kynurenic acid, 3-hydroxyisovaleric acid, vanilmandelic acid, pyroglutamic acid, 3-hydroxypropionic acid, glyceric acid, and alpha-ketoadipic acid (these are boldfaced in FIG. 6B). These 12 organic acids mostly belong to neurotransmitter and vitamin markers. Some organic acids were elevated in a subset of samples (CFS or normal), even though p value or fold change were not remarkable. No significant difference in organic acid levels was observed between CFS bedridden vs. non-bedridden, except for vanilmandelic acid (see FIGS. 6G-6I).

### 7.2.6 Viral Testing Yields Few Detectable Viruses

The MCD blood samples were also used to extract DNA which were tested for several viral infections using a real time PCR method at Coppe Laboratories, including HHV-6A and HHV-6B, HHV-7, EBV and CMV. Among the 240 participants tested with the viral assays, 9 samples showed positive results (Table 5). One CFS sample was positive for EBV, and 8 positives for HHV6 (including 7 CFS samples and 1 normal). The p-value between the CFS and control groups was p=0.074). Among the HHV6 positives, further analysis showed 4 of them were with the HHV6B genotype, and 1 of them was with the HHV6A genotype.

The frequency of viral infection in the CFS patients of our study was much lower than those reported previously (Shikova, E., et al., Cytomegalovirus, Epstein-Barr virus, and human herpesvirus-6 infections in patients with myalgic encephalomyelitis/chronic fatigue syndrome. J Med Virol, 2020). It has been reported that in ME/CFS plasma samples, EBV DNA was found in 24.l1%, CMV DNA in 3.400, and HHV-6 DNA in 1.700 of samples. On the contrary, EBV DNA was detected in 40%, and CMV and HHV-6 DNA were not found in plasma samples of normal controls (Id.). The reason for much less detectable viruses in the cohorts could be due in part to medications they take that control active viral infections. About 80% (110 CFS participants) reported having had viral or bacterial infection as a triggering event (Table 2), which is in line with what is reported in the literature.

TABLE 5
Viral Detection Tests Yield Few Detectable Viruses
Indicative of Active Ongoing Infection

| | | | | | Internal | |
|---|---|---|---|---|---|---|
| | | | | | Control | |
| | Virus | HHV6A/6B | Ct | Copies/ | RPP30 | |
| MCD ID | Detected | subtyping | Value | mL | (Ct) | Cohort |
| 100036628 | HHV6 | HHV6A | 24.29 | 229568.9 | 22.5 | Normal |
| | Detected | Detected | | | | |
| 100036589 | HHV6 | N/A | 37.21 | 47.3 | 23.63 | CFS |
| | Detected | | | | | |
| 100037181 | HHV6 | N/A | 38.84 | 16.2 | 21.12 | CFS |
| | Detected | | | | | |
| 100036608 | HHV6 | HHV6B | 24.72 | 173073.7 | 22.68 | CFS |
| | Detected | Detected | | | | |
| 100037202 | HHV6 | HHV6B | 23.94 | 288915.2 | 21.79 | CFS |
| | Detected | Detected | | | | |
| 100037159 | HHV6 | HHV6B | 24.5 | 199985.5 | 22.54 | CFS |
| | Detected | Detected | | | | |
| 100036858 | HHV6 | HHV6B | 23.07 | 511666.7 | 20.99 | CFS |
| | Detected | Detected | | | | |
| 100036599 | HHV6 | N/A | 39.38 | 11.4 | 21.08 | CFS |
| | Detected | | | | | |
| 100037041 | EBV | N/A | 37.67 | 6.7 | 24.86 | CFS |
| | Detected | | | | | |

### 7.3. Discussion

### 7.3.1 Patient Centric Approach

This study took a social media-based patient centric recruitment and at-home sample collection approach. Direct-to-patient (D32P) recruitment and remote collection of samples allowed potential enrollment of a broader study population than was not easily achievable through a conventional site-based model. Patient advocates, such as social influencers, were contacted to share resources and aid in the recruitment of participants. Advocates and influencers were also invited to enroll and participate in the study to better understand the study experience and provide context for their shareable content. D2P participants interacted with the study through a study-specific application (app), available for both iOS and Android operating systems. The app provided an end-to-end solution for deploying, completing, and managing the D2P study activities.

The D2P approach was advantageous in that it enabled participants to collect samples from geographically diverse locations without the need to set up multiple clinical sites. Participants did not need to visit a physician's office and self-collected samples at home. This was particularly convenient for CFS patients with severe symptoms who are house- or bed-bound. The MCD fingerstick whole blood collection device allows self-collection of small amounts of blood. The participants were able to simultaneously collect, and ship blood and urine samples needed for conducting multiple tests. This approach enabled the study to be conducted at a relatively low cost when compared to the anticipated costs for a site-based approach and allowed faster turn-around time. Some limitations of the approach include: 1. patient self-reported medical history was not verified. 2. discarded/missing samples due to improper collection procedure by the participants could lead to loss of samples. In this study, sample attrition rate was 3% for MCD blood samples and 8% for urine samples, which were either discarded or not received. The successful rate for sample collection was between 92-97%.

### 7.3.2 CFS and T Cell and B Cell Functions

CFS is a complex clinical condition of unknown etiology, often characterized by persistent or intermittent fatigue that is not the result of recent exertion and does not improve with rest, resulting in a significant reduction in the patient's previous normal activity. CFS is a multi-system disease, with altered immune, musculoskeletal, endocrine, neurological and cardiovascular system. In this study, 6 genes were identified using a molecular profiling approach. These genes (IKZF2, IKZF3, ABCE1, BACH2, CD3D and HSPA8) have been implicated in various biological functions, particularly in T cell and B cell biology. FIG. 7 summarizes key biological functions of these six genes.

The Ikaros zinc-finger family transcription factors (IKZF TFs) are important regulators of lymphocyte development and differentiation and are also highly expressed in B cell malignancies and are required for cancer cell growth and survival (Rivellese, F., et al., Effects of targeting the transcription factors Ikaros and Aiolos on B cell activation and differentiation in systemic lupus erythematosus. Lupus Sci Med, 2021. 8(1); Li, W., et al., The Regulatory T Cell in Active Systemic Lupus Erythematosus Patients: A Systemic Review and Meta-Analysis. Front Immunol, 2019. 10: p. 159; and Rebollo, A. and C. Schmitt, Ikaros, Aiolos and Helios: transcription regulators and lymphoid malignancies. Immunol Cell Biol, 2003. 81(3): p. 171-5). Moreover, IKZF TFs negatively control the functional properties of many immune cells. Specifically, IKZF3 plays an essential role in regulation of B-cell differentiation, proliferation and maturation to an effector state. It is involved in regulating BCL2 expression and controlling apoptosis in T-cells in an IL2-dependent manner. Diseases associated with IKZF3 include Immunodeficiency 84 and Leukemia, Chronic Lymphocytic. CD3D is a T-cell receptor/CD3 complex and is involved in T-cell development and signal transduction. IKZF2, another member of the Ikaros TFs, controls lymphocyte development, promotes quiescence, and maintains the inhibitory function of regulatory T cells, and it is frequently deleted in hypodiploid B-acute lymphoblastic leukemias (B-ALLs) (Park, S. M., et al., IKZF2 Drives Leukemia Stem Cell Self-Renewal and Inhibits Myeloid Differentiation. Cell Stem Cell, 2019. 24(1): p. 153-165 e7). BACH2 is a basic leucine zipper transcription factor expressed in B cells from the pro-B cells to mature B cells and is downregulated during the maturation to plasma cells. BACH2 is involved in primary adaptive immune response involving T cells and B cells and enables sequence-specific double-stranded DNA binding activity (Bertoni, 'F., Let's give BACH2 a breath of fresh air. Blood, 2017. 130(6): p. 696-697). BACH2 is essential for the differentiation of stem-like CD8+ T cells during chronic viral infection. Overexpression of BACH2 upregulates IKZF2 gene (Yao, C., et al., BACH2 enforces the transcriptional and epigenetic programs of stem-like CD8(+) T cells. Nat Immunol, 2021. 22(3): p. 370-380).

IKZF3 (Aiolos) and IKZF2 (Helios), and BACH2 have been implicated in other autoimmune disease such as systemic lupus erythematosus (SLE) and Rheumatoid Arthritis (RA). In this study, the expression of these genes was increased in severe cases of CFS and greater upregulation was observed in those who suffer from comorbid autoimmune diseases, suggesting that CFS is, in part, an autoimmune disease, and these genes can be utilized as potential biomarkers to distinguish mild CFS from severe CFS, with autoimmune comorbidity. Furthermore, accumulating evidence demonstrated that downregulation of IKZF3 (Aiolos) and IKZF1 (Ikaros), two members of the IKZF family, in malignant plasma cells as well as in adaptative and innate lymphocytes, is key for the anti-myeloma activity of Immunomodulatory drugs (IMiDs) (Cippitelli, M., et al., Role of Aiolos and Ikaros in the Antitumor and Immunomodulatory Activity of IMiDs in Multiple Myeloma: Better to Lose Than to Find Them. Int J Mol Sci, 2021. 22(3)). In addition, IKZF1, IKZF3 and IKZF2 (Helios) have been implicated in SLE pathogenesis. There is strong evidence that therapeutic targeting of Ikaros and Aiolos can ameliorate key pathogenic processes in human SLE (Id.).

### 7.3.3 CFS, Inflammation and Abnormal Metabolism

ME/CFS could be involved in chronic inflammation. Studies reveal several biomarkers of inflammation and a sustained immune response in the blood of ME/CFS patients (Komaroff, A. L., Inflammation correlates with symptoms in chronic fatigue syndrome. Proc Natl Acad Sci USA, 2017. 114(34): p. 8914-8916; and Blomberg, J., et al., Infection Elicited Autoimmunity and Myalgic Encephalomyelitis/Chronic Fatigue Syndrome: An Explanatory Model. Front Immunol, 2018. 9: p. 229). A few different targets of a misfiring immune system have been suggested. The presence of pro-inflammatory cytokines and several other known dysfunctions associated with ME/CFS may predispose to autoimmunity. That means autoimmune activity may be a consequence of the condition rather than a cause of it. Constant viral infections may lead to processes that could induce autoimmunity: bystander activation and molecular mimicry (Morris, G., et al., The emerging role of autoimmunity in myalgic encephalomyelitis/chronic fatigue syndrome (ME/cfs). Mol Neurobiol, 2014. 49(2): p. 741-56). A different 2013 study puts forth the possibility of an autoimmune reaction to serotonin (5-HT) (Maes, M., et al., In myalgic encephalomyelitis/chronic fatigue syndrome, increased autoimmune activity is associated with immuno-inflammatory pathways and bacterial translocation. J Affect Disord, 2013. 150(2): p. 223-30). As a hormone and neurotransmitter, serotonin performs several crucial roles in both the gut and the brain. Serotonin dysregulation has long been believed to be involved in ME/CFS. Over 60 percent of the participants with ME/CFS tested positive for autoimmune activity against 5-HT—more than 10 times the rate of the control group, and quadruple the rate of those with long-lasting fatigue that did not meet the criteria for ME/CFS (Id.).

### 7.3.4 CFS, Viral Infection and Cancer

CFS is often characterized by fatigue and severe disability. Besides fatigue, certain aspects of immune dysfunctions appear to be present in both illnesses (Meeus, M., et al., Immunological similarities between cancer and chronic fatigue syndrome: the common link to fatigue? Anticancer Res, 2009. 29(11): p. 4717-26). The underlying cause of CFS is unknown due to its heterogeneity, but in many cases it is thought to be triggered by an abnormal immune response to an agent, such as an viral infection, that results in chronic immune activation. The immunologic changes in CFS and its possible relationship with infection have prompted investigators to consider whether CFS could also be associated with an elevated risk of cancer. A 2013 study has reported that CFS was present in 0.5% of cancer cases among U.S. elderly and 0.5% of controls (Chang, C. M., J. L. Warren, and E. A. Engels, Chronic fatigue syndrome and subsequent risk of cancer among elderly US adults. Cancer, 2012. 118(23): p. 5929-36). CFS was associated with an increased risk non-Hodgkin lymphoma (NHL). Most interestingly, among NHL subtypes, CFS was associated with diffuse large B cell lymphoma, marginal zone lymphoma, and B-cell NHL not otherwise specified.

IKZF2 is highly expressed in leukemic stem cells (LSCs), and its deficiency results in defective LSC function. IKZF2 has been shown to drive Leukemia stem cell self-renewal and inhibits Myeloid differentiation. Regulation of the AML LSC program by IKZF2 thus provides a rationale to therapeutically target IKZF2 in myeloid leukemia (Park, S. M., et al., IKZF2 Drives Leukemia Stem Cell Self-Renewal and Inhibits Myeloid Differentiation. Cell Stem Cell, 2019. 24(1): p. 153-165 e7).

Abnormalities in ribonuclease (RNase) L and hyperactivation of nuclear factor kappa beta (NF-1B) are present in CFS and in prostate cancer. One of the key antiviral effectors is the IFN-inducible oligoadenylate synthetase/ribonuclease 1 (OAS/RNase L) pathway, which is activated by double-stranded RNA to synthesize unique oligoadenylates, 2-5A, to activate RNase L. RNase L exerts an antiviral effect by cleaving diverse RNA substrates, limiting viral replication; many viruses have evolved mechanisms to counteract the OAS/RNase L pathway. ATP-binding cassette E1 (ABCE1) transporter, identified as an inhibitor of RNase L, regulates RNase L activity and RNase L-induced autophagy during viral infections. In this study, ABCE1 was identified as one of the six genes upregulated in severe CFS cases. This suggests the RNase L pathway may be impaired in these individuals due to elevated ABCE1 expression. See Ramnani, B., et al., ABCE1 Regulates RNase L-Induced Autophagy during Viral Infections. Viruses, 2021. 13(2).

HSPA8 is implicated in a signal transduction pathway in the abnormal proliferation of CML cells, suggesting that the chaperone HSPA8 and CCND1 contribute to the abnormal behavior of CML cells and represent an interesting target for new therapies (Jose-Eneriz, E. S., et al., BCR-ABL1-induced expression of HSPA8 promotes cell survival in chronic myeloid leukaemia. Br J Haematol, 2008. 142(4): p. 571-82).

Clinical activity from B-cell depletion using anti-CD20 antibody Rituximab has been demonstrated in treating CFS patients (Fluge, O., et al., Benefit from B-lymphocyte depletion using the anti-CD20 antibody rituximab in chronic fatigue syndrome. A double-blind and placebo-controlled study. PLoS One, 2011. 6(10): p. e26358). However, improvement of fatigue was observed in the responders 3 to 7 months after the treatment and the initial rapid B-cell depletion. This data suggested that CFS may be an autoimmune disease, and the delayed response to Rituximab could be due to the elimination of the disease-associated autoantibodies. It is also speculated that this response pattern could be related to interaction of B-cell and T-cell in antigen presentation. The finding herein of up-regulation of both B-cell and T-cell associated genes support this hypothesis.

### 7.3.5 CFS and Long-Hauler COVID

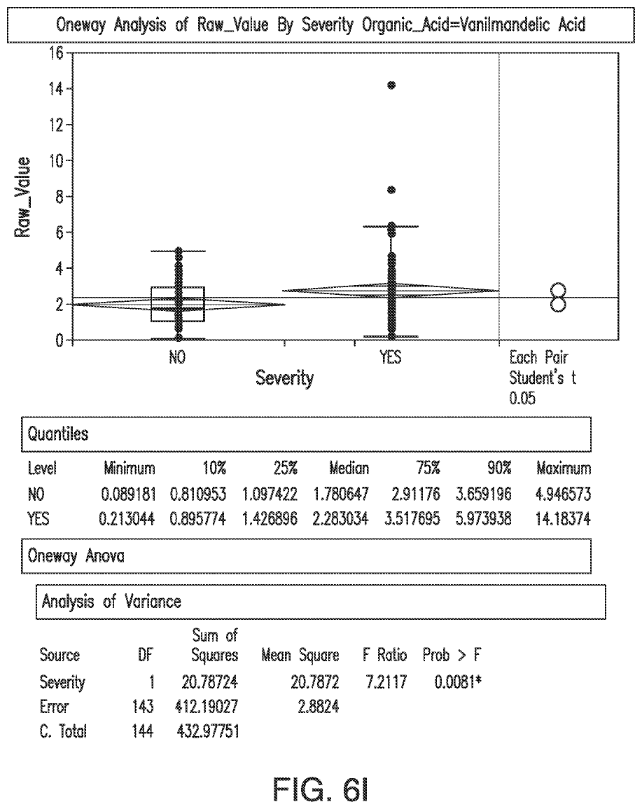

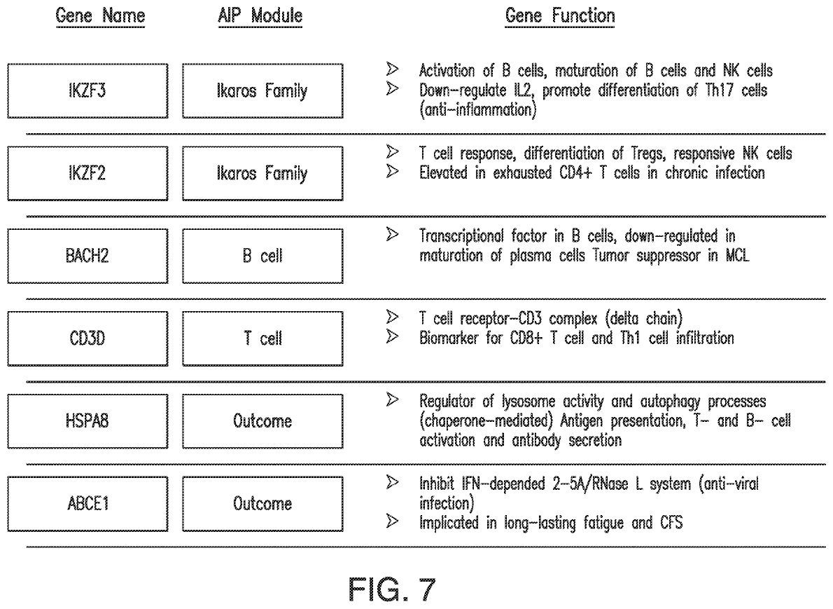

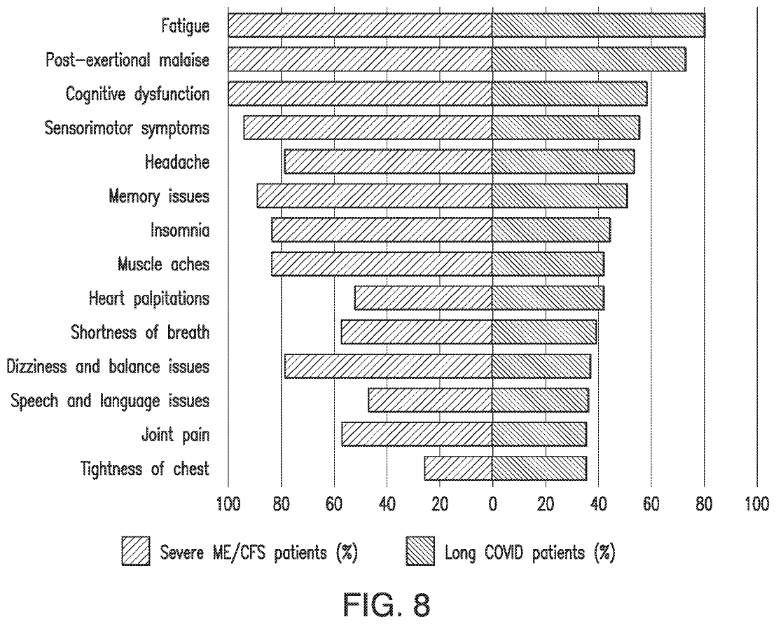

The link between the immune system and CFS has been explored and is supported by the coincidence of the onset of symptoms with viral infections (Galbraith, S., et al., Peripheral blood gene expression in post-infective fatigue syndrome following from three different triggering infections. J Infect Dis, 2011. 204(10): p. 1632-40), and the beneficial effect of treatment of human herpesvirus 6 and Epstein-Bar virus infection in CFS symptoms (Watt, T., et al., Response to valganciclovir in chronic fatigue syndrome patients with human herpesvirus 6 and Epstein-Barr virus IgG antibody titers. J Med Virol, 2012. 84(12): p. 1967-74). There was a striking similarity in symptoms between long hauler COVID-19 cases and ME/CFS (FIG. 8), suggesting that studies on the mechanism and treatment of ME/CFS may help prevent and treat long COVID and vice versa (Chang, C. J., et al., A Comprehensive Examination of Severely Ill ME/CFS Patients. Healthcare (Basel), 2021. 9(10); Campen, C., P. C. Rowe, and F. C. Visser, Orthostatic Symptoms and Reductions in Cerebral Blood Flow in Long-Haul COVID-19 Patients: Similarities with Myalgic Encephalomyelitis/Chronic Fatigue Syndrome. Medicina (Kaunas), 2021. 58(1); Davis, H. E., et al., Characterizing long COVID in an)international cohort: 7 months of symptoms and their impact. EClinicalMedicine, 2021. 38: p. 101019; and Hunt, J., et al., Long Covid at the crossroads: Comparisons and lessons from the treatment of patients with myalgic encephalomyelitis/chronic fatigue syndrome (ME/CFS). J Health Psychol, 2022: p. 13591053221084494). Before the pandemic, estimated 3 million Americans were suffering from ME/CFS. An unprecedented wave of CFS-like illness related to COVID might appear over the next few years, with profound societal costs. Accordingly, studies such as this one that identify biomarkers associated with fatigue have heightened importance.

An estimated 10% of people who have been infected with SARS-CoV-2 can experience long-term effects known as long COVID. It is unclear what causes of CFS or long COVID. The present disclosure discovered that both conditions appeared to follow an infection, and had striking similarities in symptoms. In this study, both acute COVID and long COVID patient samples were profiled along with respective healthy controls using DxTerity's AIP gene expression test. The six genes identified in the CFS study were significantly downregulated in the acute COVID cohort, when comparing to the healthy cohort (FIGS. 9A-9F). This trend, however, changed in the long COVID cohort. These genes were expressed slightly greater than the healthy cohort (except for IKZF2) (FIGS. 10A-10F). The pattern of expression level changed from acute to long COVID was very similar to what was observed in the CFS study. This could be biologically significant, as the immune system is initially hit hard with acute infection which dramatically represses certain gene expressions. The immune system continues to be activated, but over time in most people it would return to baseline. However, in the long COVID cases, chronic conditions may lead over-compensation of the immune system and up-regulation of certain gene expressions such as exhibited in these long COVID patients.

The study suggested that the use of social media for patient recruitment and the use of at-home sample collection represent a novel approach for conducting clinical research which saves cost and time and removes the need for office visit. Further, gene expression and metabolic profiles were used to identify biomarkers associated with CFS or subsets of CFS patients with different disease severity, which can be used for improved disease diagnosis and treatment.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method of identifying a subject having Chronic Fatigue Syndrome (CFS) or verifying CFS in a subject, the method comprising:
(a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; and
(b) identifying or verifying the subject as having CFS if the expression level of the biomarker is higher than a reference expression level of the biomarker.

2. The method of claim 1, wherein the subject has reported chronic debilitating fatigue, unrefreshing sleep, mental and/or physical pain, neurological and cognitive impairment, and/or autoimmunity or immunodeficiencies.

3. A method of determining severity of CFS in a subject, the method comprising:
(a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D;
(b) comparing the expression level of the biomarker with a reference expression level of the biomarker; and
(c) determining the severity of CFS in the subject based on the comparison in step (b).

4. The method of claim 3, wherein the severity of CFS is determined to be severe if the expression level of the biomarker is higher than the reference expression level of the biomarker, optionally wherein the reference expression level of the biomarker is the expression level of the biomarker in a subject having mild CFS or a cohort of subjects having mild CFS.

5. A method of identifying a subject who is likely or not likely to be responsive to a treatment of CFS or predicting the responsiveness of a subject to a treatment of CFS, the method comprising:
(a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D; and
(b) identifying or predicting the subject as being likely to be responsive to the treatment if the expression level of the biomarker is higher than a reference expression level of the biomarker.

6. The method of claim 5, wherein the method further comprises administering the treatment to the subject identified or predicted to be likely to be responsive to the treatment.

7. A method of selectively treating a subject having or suspected of having CFS with a treatment, the method comprising:
(a) determining an expression level of a biomarker in a sample from the subject, wherein the biomarker is a cereblon (CRBN)-associated protein (CAP) selected from the group consisting of HSPA8, ABCE1, IKZF2, IKZF3, BACH2 and CD3D;
(b) identifying or predicting the subject as being likely to be responsive to the treatment of CFS if the expression level of the biomarker is higher than a reference expression level of the biomarker; and
(c) administering the treatment to the subject identified or predicted to be likely to be responsive to the treatment.

8. The method of any one of claims 1-7, wherein the reference expression level of the biomarker is a predetermined expression level of the biomarker.

9. The method of any one of claims 1-8, wherein the reference expression level of the biomarker is the expression level of the biomarker in a subject who does not have CFS or a cohort of subjects not having CFS.

10. The method of any one of claims 1-8, wherein the reference expression level of the biomarker is the expression level of the biomarker in a healthy subject or a cohort of healthy subjects.

11. The method of any one of claims 1-8, wherein the reference expression level of the biomarker is the expression level of the biomarker in a subject having mild CFS or a cohort of subjects having mild CFS.

12. The method of any one of claims 1-11, wherein the biomarker is HSPA8 or ABCE1, optionally wherein the reference expression level of the biomarker is the expression level of the biomarker in a healthy subject or a subject who does not have CFS, or a cohort of healthy subjects or subjects not having CFS.

13. The method of any one of claims 1-12, wherein the method comprises determining the expression levels of two, three, four, five, or all biomarkers selected from the group consisting of HSPA8, ABCE1, IKZF2, IKZF3, BACH2 and CD3D.

14. The method of claim 13, wherein the method comprises determining the expression levels of:
(i) IKZF2 and at least one, two, three or four of IKZF3, ABCE1, BACH2, CD3D and HSPA8;
(ii) IKZF3 and at least one, two, three or four of IKZF2, ABCE1, BACH2, CD3D and HSPA8;
(iii) ABCE1 and at least one, two, three or four of IKZF2, IKZF3, BACH2, CD3D and HSPA8;
(iv) BACH2 and at least one, two, three or four of IKZF2, IKZF3, ABCE1, CD3D and HSPA8;
(v) CD3D and at least one, two, three or four of IKZF2, IKZF3, ABCE1, BACH2, and HSPA8; or
(vi) HSPA8 and at least one, two, three or four of IKZF2, IKZF3, ABCE1, BACH2, and CD3D.

15. The method of claim 13, wherein the method comprises determining the expression levels IKZF2, IKZF3, ABCE1, BACH2, CD3D and HSPA8.

16. The method of any one of claims 13-15, wherein the method comprises comparing the expression level of each of the biomarkers with their respective reference expression level.

17. The method of any one of claims 13-15, wherein the method comprises obtaining a composite score based on the expression levels of the biomarkers and comparing the composite score with a reference score derived from the reference expression levels of the biomarkers.

18. A method of determining or monitoring effectiveness of a treatment in a subject having CFS, the method comprising:
(a) determining a first expression level of a biomarker in a first sample obtained from the subject before administering the treatment to the subject, wherein the biomarker is a cereblon (CRBN)-associated protein (CAP) selected from the group consisting of HSPA8, ABCE1, IKZF2, IKZF3, BACH2 and CD3D;
(b) administering the treatment to the subject;
(c) determining a second expression level of the biomarker in a second sample obtained from the subject after administering the treatment to the subject; and
(d) determining the effectiveness of the treatment based on the comparison of the first expression level with the second expression level.

19. The method of claim 18, wherein the method comprises determining that the treatment is effective if the second expression level is lower than the first expression level.

20. The method of claim 18 or 19, wherein the method further comprises determining or adjusting a dose of the treatment to the subject.

21. A method of screening a treatment for effectiveness in treating CFS, the method comprising:
(a) determining a first expression level of a biomarker in a sample before administering the treatment to the sample, wherein the biomarker is a cereblon (CRBN)-associated protein (CAP) selected from the group consisting of HSPA8, ABCE1, IKZF2, IKZF3, BACH2 and CD3D;
(b) administering the treatment to the sample;
(c) determining a second expression level of the biomarker in the sample after administering the treatment to the sample;
(d) comparing the first expression level with the second expression level; and
(e) selecting the treatment if the second expression level is lower than the first expression level.

22. The method of any one of claims 18-21, wherein the biomarker is HSPA8 or ABCE1.

23. The method of any one of claims 18-22, wherein the method comprises determining the first and second expression levels of two, three, four, five, or all biomarkers selected from the group consisting of HSPA8, ABCE1, IKZF2, IKZF3, BACH2 and CD3D.

24. The method of claim 23, wherein the method comprises determining the first and second expression levels of:
(i) IKZF2 and at least one, two, three or four of IKZF3, ABCE1, BACH2, CD3D and HSPA8;
(ii) IKZF3 and at least one, two, three or four of IKZF2, ABCE1, BACH2, CD3D and HSPA8;
(iii) ABCE1 and at least one, two, three or four of IKZF2, IKZF3, BACH2, CD3D and HSPA8;
(iv) BACH2 and at least one, two, three or four of IKZF2, IKZF3, ABCE1, CD3D and HSPA8;
(v) CD3D and at least one, two, three or four of IKZF2, IKZF3, ABCE1, BACH2, and HSPA8; or
(vi) HSPA8 and at least one, two, three or four of IKZF2, IKZF3, ABCE1, BACH2, and CD3D.

25. The method of claim 23, wherein the method comprises determining the first and second expression levels of all biomarkers in the group consisting of HSPA8, ABCE1, IKZF2, IKZF3, BACH2 and CD3D.

26. The method of any one of claims 23-25, wherein the method comprises comparing the first expression level of each of the biomarkers with their respective second expression level.

27. The method of any one of claims 23-25, wherein the method comprises obtaining a first composite score based on the first expression levels of the biomarkers and a second composite score based on the second expression level of the biomarkers, and comparing the first composite score with the second composite score.

28. The method of any one of claims 5-27, wherein the treatment comprises an immunomodulatory drug (IMiD).

29. The method of any one of claims 5-27, wherein the treatment comprises a celebron (CRBN) modulator or a compound capable of binding and/or inducing conformational change to CRBN.

30. The method of any one of claims 5-27, wherein the treatment comprises an agent that depletes B cells.

31. The method of any one of claims 1-30, wherein the CFS is associated with an autoimmune disease or a viral infection.

32. A method of identifying a subject having long COVID or verifying long COVID in a subject, the method comprising:
(a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and
(b) identifying or verifying the subject as having long COVID if the expression level of the biomarker is higher than a reference expression level of the biomarker.

33. A method of identifying a subject who is likely or not likely to be responsive to a treatment of long COVID or predicting the responsiveness of a subject to a treatment of long COVID, the method comprising:
(a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D; and
(b) identifying or predicting the subject as being likely to be responsive to the treatment if the expression level of the biomarker is higher than a reference expression level of the biomarker.

34. The method of claim 33, wherein the method further comprises administering the treatment to the subject identified or predicted to be likely to be responsive to the treatment.

35. A method of selectively treating a subject having or suspected of having long COVID with a treatment, the method comprising:
(a) determining an expression level of a biomarker in a sample from the subject, wherein the biomarker is a cereblon (CRBN)-associated protein (CAP) selected from the group consisting of HSPA8, IKZF3, ABCE1, IKZF2, BACH2 and CD3D;
(b) identifying or predicting the subject as being likely to be responsive to a treatment of long COVID if the expression level of the biomarker is higher than a reference expression level of the biomarker; and
(c) administering the treatment to the subject identified or predicted to be likely to be responsive to the treatment.

36. The method of any one of claims 32-35, wherein the reference expression level of the biomarker is a predetermined expression level of the biomarker.

37. The method of any one of claims 32-35, wherein the reference expression level of the biomarker is the expression level of the biomarker in a subject who does not have long COVID or a cohort of subjects not having long COVID.

38. The method of any one of claims 32-35, wherein the reference expression level of the biomarker is the expression level of the biomarker in a healthy subject or a cohort of healthy subjects.

39. The method of any one of claims 32-35, wherein the reference expression level of the biomarker is the expression level of the biomarker in a subject having acute COVID or a cohort of subjects having acute COVID.

40. The method of any one of claims 32-39, wherein the biomarker is HSPA8 or IKZF3, optionally wherein the reference expression level of the biomarker is the expression level of the biomarker in a healthy subject or a subject who does not have long COVID, or a cohort of healthy subjects or subjects not having long COVID.

41. The method of any one of claims 32-39, wherein the method comprises determining the expression levels of two, three, four, five, or all biomarkers selected from the group consisting of HSPA8, IKZF3, ABCE1, IKZF2, BACH2 and CD3D.

42. The method of claim 41, wherein the method comprises determining the expression levels of:
(i) IKZF2 and at least one, two, three or four of IKZF3, ABCE1, BACH2, CD3D and HSPA8;
(ii) IKZF3 and at least one, two, three or four of IKZF2, ABCE1, BACH2, CD3D and HSPA8;
(iii) ABCE1 and at least one, two, three or four of IKZF2, IKZF3, BACH2, CD3D and HSPA8;
(iv) BACH2 and at least one, two, three or four of IKZF2, IKZF3, ABCE1, CD3D and HSPA8;
(v) CD3D and at least one, two, three or four of IKZF2, IKZF3, ABCE1, BACH2, and HSPA8; or
(vi) HSPA8 and at least one, two, three or four of IKZF2, IKZF3, ABCE1, BACH2, and CD3D.

43. The method of claim 41, wherein the method comprises determining the expression levels HSPA8, IKZF3, ABCE1, IKZF2, BACH2 and CD3D.

44. The method of any one of claims 41-43, wherein the method comprises comparing the expression level of each of the biomarkers with their respective reference expression level.

45. The method of any one of claims 41-43, wherein the method comprises obtaining a composite score based on the expression levels of the biomarkers and comparing the composite score with a reference score derived from the reference expression levels of the biomarkers.

46. A method of determining or monitoring effectiveness of a treatment in a subject having long COVID, the method comprising:
(a) determining a first expression level of a biomarker in a first sample obtained from the subject before administering the treatment to the subject, wherein the biomarker is a cereblon (CRBN)-associated protein (CAP) selected from the group consisting of HSPA8, IKZF3, ABCE1, IKZF2, BACH2 and CD3D;
(b) administering the treatment to the subject;
(c) determining a second expression level of the biomarker in a second sample obtained from the subject after administering the treatment to the subject; and
(d) determining the effectiveness of the treatment based on the comparison of the first expression level with the second expression level.

47. The method of claim 46, wherein the method comprises determining that the treatment is effective if the second expression level is lower than the first expression level.

48. The method of claim 46 or 47, wherein the method further comprises determining or adjusting a dose of the treatment to the subject.

49. A method of screening a treatment for effectiveness in treating long COVID, the method comprising:
(a) determining a first expression level of a biomarker in a sample before administering the treatment to the sample, wherein the biomarker is a cereblon (CRBN)-associated protein (CAP) selected from the group consisting of HSPA8, IKZF3, ABCE1, IKZF2, BACH2 and CD3D;
(b) administering the treatment to the sample;
(c) determining a second expression level of the biomarker in the sample after administering the treatment to the sample;
(d) comparing the first expression level with the second expression level; and
(e) selecting the treatment if the second expression level is lower than the first expression level.

50. The method of any one of claims 46-49, wherein the biomarker is HSPA8 or IKZF3.

51. The method of any one of claims 46-50, wherein the method comprises determining the first and second expression levels of two, three, four, five, or all biomarkers selected from the group consisting of HSPA8, IKZF3, ABCE1, IKZF2, BACH2 and CD3D.

52. The method of claim 51, wherein the method comprises determining the first and second expression levels of:
(i) IKZF2 and at least one, two, three or four of IKZF3, ABCE1, BACH2, CD3D and HSPA8;
(ii) IKZF3 and at least one, two, three or four of IKZF2, ABCE1, BACH2, CD3D and HSPA8;
(iii) ABCE1 and at least one, two, three or four of IKZF2, IKZF3, BACH2, CD3D and HSPA8;
(iv) BACH2 and at least one, two, three or four of IKZF2, IKZF3, ABCE1, CD3D and HSPA8;
(v) CD3D and at least one, two, three or four of IKZF2, IKZF3, ABCE1, BACH2, and HSPA8; or
(vi) HSPA8 and at least one, two, three or four of IKZF2, IKZF3, ABCE1, BACH2, and CD3D.

53. The method of claim 51, wherein the method comprises determining the first and second expression levels of all biomarkers in the group consisting of HSPA8, IKZF3, ABCE1, IKZF2, BACH2 and CD3D.

54. The method of any one of claims 51-53, wherein the method comprises comparing the first expression level of each of the biomarkers with their respective second expression level.

55. The method of any one of claims 51-53, wherein the method comprises obtaining a first composite score based on the first expression levels of the biomarkers and a second composite score based on the second expression level of the biomarkers, and comparing the first composite score with the second composite score.

56. The method of any one of claims 33-55, wherein the treatment comprises an immunomodulatory drug (IMiD).

57. The method of any one of claims 33-55, wherein the treatment comprises a CRBN modulator or a compound capable of binding and/or inducing conformational change to CRBN.

58. The method of any one of claims 33-55, wherein the treatment comprises an agent that depletes B cells.

59. The method of any one of claims 32-58, wherein the subject has had Coronavirus Disease 2019 (COVID-19).

60. The method of any one of claims 1-59, wherein the expression level of the biomarker is determined by measuring the mRNA level of the biomarker.

61. The method of claim 60, wherein the mRNA level is determined by using quantitative reverse-transcriptase PCR (RT-qPCR), microarray, Northern blot or RNA sequencing.

62. The method of any one of claims 1-59, wherein the expression level of the biomarker is determined by measuring the protein level of the biomarker.

63. The method of claim 62, wherein the protein level of the biomarker is determined by using mass spectrometry (MS), liquid chromatography-tandem mass spectrometry (LC MS/MS), immunoassay, flow cytometry, immunohistochemistry, western blot, or enzyme-linked immunosorbent assay (ELISA).

64. A kit for performing the method of any one of claims 1-63, the kit comprising an agent for determining the expression level of at least one biomarkers selected from the group consisting of HSPA8, IKZF3, ABCE1, IKZF2, BACH2 and CD3D.

65. The kit of claim 64, wherein the kit further comprises a tool for obtaining the sample.

66. The kit of claim 64 or 65, wherein the kit further comprises an instruction on interpreting the determined expression level.

67. The kit of any one of claims 64-66, wherein the kit further comprises the reference expression level of the biomarker.
